(19) United States
(12) Patent Application Publication
(10) Pub. No.: US 2024/0307316 A1
(43) Pub. Date: Sep. 19, 2024

(54) ANTIOXIDANT AND OXYGEN-RELEASING LIGNIN COMPOSITES TO ACCELERATE WOUND HEALING

(71) Applicants: Board of Supervisors of Louisiana State University and Agricultural and Mechanical College, Baton Rouge, LA (US); Inha University, Michuhol-gu, Incheon (KR); Baylor College of Medicine, Houston, TX (US)

(72) Inventors: Jangwook P. Jung, Baton Rouge, LA (US); Swathi Balaji, Houston, TX (US); Myungwoong Kim, Incheon (KR); Carlos E. Astete, Baton Rouge, LA (US)

(21) Appl. No.: 18/605,959

(22) Filed: Mar. 15, 2024

Related U.S. Application Data
(60) Provisional application No. 63/490,275, filed on Mar. 15, 2023.

(51) Int. Cl. A61K 9/48; A61K 33/08; A61P 17/02

(52) U.S. Cl. CPC A61K 9/4866; A61K 9/4825; A61K 33/08; A61P 17/02

(57) ABSTRACT

In one aspect, the disclosure relates to compounds comprising one or more lignin derivatives, at least one organic peroxide, and a pharmaceutically-acceptable carrier. In an aspect, the one or more lignin derivatives can be or include thiolated lignosulfonate (TLS), sodium lignosulfonate (SLS), or a combination thereof. In one aspect, the SLS can be grafted to a polymer or copolymer such as poly(lactic-co-glycolic) acid (PLGA) in order to form a shell of a core-shell nanoparticle, wherein the shell surrounds a core of the inorganic peroxide and protects the core from premature activation in an aqueous wound environment. In another aspect, the pharmaceutically-acceptable carrier can be a hydrogel forming polymer such as a methacrylated gelatin, which can assist in modulating the properties of the lignin derivatives to be suitable for injection. Also disclosed herein are methods of using the disclosed compositions for the treatment of wounds in subjects, including in diabetic subjects.

### CROSS REFERENCE TO RELATED APPLICATIONS

This application claims the benefit of U.S. Provisional Application No. 63/490,275 filed on Mar. 15, 2023, which is incorporated herein by reference in its entirety.

### STATEMENT REGARDING FEDERALLY SPONSORED RESEARCH OR DEVELOPMENT

This invention was made with government support under grant number OIA 1632854 awarded by the National Science Foundation. The government has certain rights in the invention.

### BACKGROUND

Over eight million people suffer from non-healing wounds every year in the United States. Chronic wounds with impeded healing occur either due to infection or underlying conditions such as obesity, diabetes, or aging, which are on the rise. Recurrence of wounds in diabetic patients due to poor healing significantly increases their morbidity, risk for amputations, and mortality, and diabetes-related lower extremity complications are among the top 10 leading causes of the global burden of disability.

Wound healing is a critical process which progresses through tightly regulated phases and ultimately leads to repopulation of the wound with cells and extracellular matrix (ECM) to repair the injured site. A key aspect of the wound healing process involves the production of granulation tissue, a densely vascularized provisional tissue composed of fibroblasts (FBs), vascular endothelial cells (ECs), inflammatory cells and cell-derived ECM. Poor vascularization of the granulation tissue is often associated with impaired healing. Recent evidence further links excessive production of reactive oxygen species (ROS) and/or impaired detoxification of ROS to the pathogenesis of impaired wound healing. Excess ROS accumulation disrupts cellular homeostasis and causes non-specific damage to critical cellular components and function, leading to impairment such as abhorrent FB collagen synthesis and cell apoptosis, EC and smooth muscle cell dysfunction, compromised tissue perfusion, and increased proinflammatory cytokine secretion by macrophages. Excess ROS is scavenged by enzymes, such as superoxide dismutase and antioxidants, which regulate the redox environment in healing skin wounds.

While acute, physiologic wounds progress through a series of wound healing stages of coagulation, inflammation, migration and proliferation, and remodeling, diabetic wounds are known to deviate from this wound healing pattern. Diabetic wounds have a reduced ability to mount the effective immune response required for pathogen control due to reduced migration of leukocytes, neutrophils, and macrophages to the wound. The accumulation of advanced glycation end products in diabetic wounds increases ROS formation and reduction of macrophage efferocytosis, thereby impinging their ability to transition to M2 (alternatively activated) phenotype and impairing inflammation resolution. This results in increased proteolytic activity along with a decrease in proteolysis inhibitors, culminating in insufficient accumulation of granulation tissue and neovascularization. Recent evidence further links excessive production of ROS and/or impaired detoxification of ROS to the pathogenesis of chronic wounds.

In recent years, antioxidants have drawn much attention as potential therapeutic interventions due to their ability to fight oxidative stress. The main function of antioxidants is to scavenge or neutralize free radical formation and to inhibit the deleterious downstream effects of ROS. However, most antioxidants, taken orally, have limited absorption profiles, which leads to low bioavailability and insufficient concentrations at the target site. To overcome this issue, research has been focused on developing tissue engineering strategies to provide locoregional delivery of antioxidants. Strategies including antioxidant nanoparticles made of inorganic materials, such as mesoporous silica, cerium oxide, and fullerene, have been evaluated in in vitro assays and in animal models to determine their ability to scavenge free radicals while decreasing ROS concentrations to protect cells against oxidative stress. Hydrogels that release ROS scavengers have been developed to promote cell function as a method to mitigate the foreign body response. Additionally, decellularized myocardial matrix has shown to protect cardiomyocytes from ROS after myocardial infarction. However, currently utilized biomaterials, especially natural biomaterials, employ relatively complex chemistry and lack mechanical tunability.

Toward the goal of tissue regeneration, ROS-responsive biomaterials have been identified as a type of promising therapeutic avenue to alleviate oxidative stress in tissue microenvironments. Engineered biomaterials can also address the issue of the wound exudates that make healing a challenge, because incessant release of elevated levels of exudates in chronic wounds promote microbial infection and free radicals that oxidize biomolecules and activate the inflammatory-ECM production cascades. It has been shown that hydrogels create a provisional wound matrix with good biocompatibility, nutrient supply, and swelling that allows absorption of excess exudates and maintenance of optimal moisture. Thus, appropriate design of these biomaterials renders resistance to excess wound hydrolysis and can provide missing cues such as antioxidation and ROS scavenging, thereby jump-starting healing by redirecting the wounds from the state of inflammation to the next stages. Furthermore, when large wounds are considered, lack of nutrient supply and oxygen generation beyond the limits of diffusion in tissue (100 to 200 μm) have been a major limiting factor for biomaterial-based therapies. These deficiencies and the simultaneous disruption of several pathways involved in diabetic wound healing response may in part explain why some of the current therapies to treat diabetic wound healing are not entirely successful.

Lignin is a polyphenolic polymer that functions in plants to isolate pathogens to the site of infection while providing impermeability to cell walls in vascular plants. The use of lignin in wound-healing dressings is an emerging field, with proven effects as an antiviral, and for its antioxidation, immunostimulation, and anticoagulation properties. In addition, the enhanced mechanical properties with good protein adsorption capacity and wound compatibility of this natural polyphenolic molecule, along with its anti-inflammatory properties, such as its capacity to reduce gene expression of iNOS (inducible nitric oxide synthase) and IL-1B (interleukin-1B) of inflamed macrophages, and enhanced biocompatibility without toxicity are promising for wound healing applications. Previous studies showed that lignin treatment improved mechanical properties and wound healing of incision wounds in Sprague-Dawley rats. Not surprisingly, the majority of applications of lignin-based biomaterials take advantage of its antioxidation property. Review of relevant literature shows that the primary mode of application of lignin to wounds is via entrapment or complexation for rapid release of nanoparticles. However, research thus far has not addressed compositions containing tunable mixtures of functionalized lignin derivatives acting synergistically to improve wound healing.

In a small scale oxygenation, many solid inorganic peroxides have been used to support cell growth, survival, tissue regeneration, and bioremediation. Calcium peroxide (CaO₂) possesses many distinctive properties in comparison with other peroxides, including better thermal stability, environmental harmless end products, extended release period of hydrogen peroxide, and reasonable cost. Based on the relatively low solubilities (CaO₂ 1.65 mg/ml at 20° C. and MgO₂ 0.86 mg/mL at 18° C.), calcium peroxide has a higher oxygen generation potential than magnesium peroxide.

Despite advances in wound healing research, there is still a scarcity of systems that are potent and effective in detoxification of excessive levels of ROS while still enabling a level activity of ROS necessary for normal healing functions. Ideal compositions and methods could be administered locally and/or regionally for more effective delivery of active ingredients, including antioxidants, and would promote neovascularization and granulation tissue deposition while reducing inflammation. These needs and other needs are satisfied by the present disclosure.

### SUMMARY

In accordance with the purpose(s) of the present disclosure, as embodied and broadly described herein, the disclosure, in one aspect, relates to compounds comprising one or more lignin derivatives, at least one organic peroxide, and a pharmaceutically-acceptable carrier. In an aspect, the one or more lignin derivatives can be or include thiolated lignosulfonate (TLS), sodium lignosulfonate (SLS), or a combination thereof. In one aspect, the SLS can be grafted to a polymer or copolymer such as, for example, poly(lactic-co-glycolic) acid (PLGA) in order to form a shell of a core-shell nanoparticle, wherein the shell surrounds a core of the inorganic peroxide and protects the inorganic peroxide from premature activation in an aqueous wound environment. In another aspect, the pharmaceutically-acceptable carrier can be a hydrogel forming polymer such as, for example, a methacrylated gelatin, which can assist in modulating the properties of the lignin derivatives to be suitable for injection or administration as droplets or aerosolized formulations. Also disclosed herein are methods of using the disclosed compositions for the treatment of wounds and other conditions and symptoms associated with excessive ROS, inflammation, and lack of neovascularization in subjects, including in, but not limited to, diabetic subjects.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show a schematic and assessment of SLS thiolation. (FIG. 1A) Thiolation of SLS to form TLS. (FIG. 1B) ³¹P NMR (nuclear magnetic resonance) (in CDCl₃) spectra of SLS or TLS via TMDP (2-chloro-4,4,5,5-tetramethyl-1,3,2-dioxaphospholane) hydrolysis. (FIG. 1C) Thiol concentrations of TLS samples prepared under different stoichiometry determined by Ellman's assay. [MPA] is defined as the number of moles (mmol) of added MPA per 0.1 gram of SLS; ** p<0.01, ANOVA (analysis of variance) Dunnett's post hoc test, mean±standard deviation (SD), n=3.

FIG. 2 shows molecular weight distribution of alkali-extracted lignins and SLS by gel permeation chromatography (GPC). Weight-averaged and number-average molecular weights and respective polydispersity index (PDI) are reported in Table 1. The Mw distribution of SG and SLS showed that the secondary peak of SG and the primary peak of SLS are overlapped around 250-300 g/mol.

FIGS. 3A-3F show transmission electron micrographs of SLS in solution (0.1 mg/mL, negative staining with 3% phosphotungstic acid (PTA) negative stain (FIGS. 3A-3B). Collagen-SLS composite in solution, 0.1 mg/mL+0.278 mg/mL, using a uranyl acetate (UA) negative stain (FIGS. 3C-3D). Collagen fibrils in solution, 0.1 mg/mL, using a UA stain (FIGS. 3E-3F).

FIG. 4 shows ¹H NMR (D₂O) spectra of gelatin and GelMA (methacrylated gelatin). Coupling of methacrylic acid (MA) to gelatin was quantified from the integrated peak intensities of protons in phenyl group (a), alkene (b) and carbon adjacent to primary amine of lysine (c).

FIG. 5 shows elemental analysis of nanoparticles (NPs) of SLS-PLGA with or without CaO₂ by microPXTE. The concentration (arbitrary units, a.u.) of Ca, Cl, P, S, and K in the NPs normalized to the NPs of TLS. TLS also had trace amounts of Mn and Fe. The bars represent counting uncertainties (1σ) in a single measurement analyzed by GeoPIXE software; mean±SEM.

FIG. 6 shows quantification of O₂ from lignin composites with NPs of SLS-PLGA/CaO₂. Area under curve (AUC) is calculated and the differential between CPO and CPOc lignin composites over 1440 min is reported (see Table 3 for abbreviations for lignin compositions). One-way ANOVA with Tukey's post hoc test, no significant difference from each other; mean±SD, n=4.

FIGS. 7A-7D show swelling ratios and degradation of CPO and CPOc lignin composites. (FIG. 7A) Swelling ratios normalized to TLS lignin composites. No statistical difference between CPO and CPOc lignin composites at the same concentration and between two different concentrations at each composite type. Student's t-test shows no statistically significant difference; mean±SD, n=3. Degradation of lignin composites, TLS (FIG. 7B), CPO (FIG. 7C) and CPOc (FIG. 7D), in the solution of 0.5 U/mL collagenase (enzyme) or serum-free (SF) medium. In (FIGS. 7B-7D), symbols represent the concentrations of NPs (SLS-PLGA with or without CaO₂) at 4 and 40 mg/mL, respectively; mean±SD, n=3.

FIGS. 8A-8D show oscillating rheometry of lignin composites. (FIG. 8A) Viscosity of each precursor before photo-crosslinking. (FIG. 8B) Axial stresses are plotted against compression varying from 0 to 20%. (FIG. 8C) Frequency sweeping of lignin composites. Solid and open symbols represent G′ (storage modulus) and G″ (loss modulus), respectively. FIG. 8D) Loss tangent (δ) of lignin composites from 0.1 to 10 rad/s. mean±SD, n=3 for all samples.

FIGS. 9A-9C show morphometric analysis of wounds treated with lignin composites at 7 days post-wounding. Wounds in WT C57BL/6N mice were treated with lignin composites (FIG. 9A) to measure epithelial gap (FIG. 9B) and granulation tissue area (FIG. 9C). In (FIG. 9A), hematoxylin (nuclei) and eosin (ECM and cytoplasm, H&E) stained wound sections from different treatments are shown. The left panels show the cross section of the wounds from edge to edge, and the right panels show corresponding higher magnification of boxed areas (inset) of the granulating wound bed with biomaterial interface. Scale bar, 100 μm. (FIG. 9B) Quantification of epithelial gap and granulations tissue area are shown. One-way ANOVA with Kruskal-Wallis test followed by Dunn's multiple comparison test was performed. * p<0.05 and ** p<0.0 1.4≤n≤7, bar plots indicate mean±SD, with individual values from each mouse wound indicated. Details of compositions of UNTX, TLS, CPOc and CPO are in Table 3.

FIGS. 10A-10E show assessment of neovascularization in the wounds treated with lignin composites at 7 days post wounding. Wounds in WT C57BL/6N mice were treated with lignin composites and the extent of neovascularization was assessed with immunostaining (CD31) and hematoxylin counterstaining (nuclei). CD31+ cells (FIG. 10B) and vessel formation (FIG. 10C) per high powered field (HPF) were quantified. The infiltration of aSMA⁺ cells in the wounds is visualized (FIG. 10D) and quantified per HPF (FIG. 10E). Scale bars, 50 μm in (FIG. 10A) and 3 mm in (FIG. 10D), respectively. One-way ANOVA with Tukey's post hoc tests, ** p<0.01 and *p<0.05. 3≤n≤6, bar plots indicate mean±SD with individual values per each mouse wound indicated. Details of compositions of UNTX, TLS, CPOc and CPO are in Table 3.

FIG. 11A-11E show assessment of inflammatory responses in the wounds treated with lignin composites at 7 days post wounding. Wounds in WT C57BL/6N mice were treated with lignin composites and the extent of inflammatory responses were assessed (FIG. 11A) with immunohistochemical staining and hematoxylin counterstaining (nuclei). CD45⁺ leukocytes (FIG. 11B), Ly6G⁺ monocytes, granulocytes, and neutrophils (FIG. 11C), F4/80⁺ pan macrophages (FIG. 11D) and CD206 M2 macrophages (FIG. 11E) per HPF are quantified. In (FIG. 11A), black scale bars, 50 μm and grey scale bars, 125 μm, respectively. 3≤n≤6, bar plots indicate mean±SD with individual values per each mouse wound indicated. Details of compositions of UNTX, TLS, CPOc and CPO are in Table 3.

FIGS. 12A-12C show scar assessment in the wounds treated with lignin composites at 28 days after surgery. Wound sections from WT C57BL/6N mice treated with lignin composites were stained with trichrome (collagen), and the collagen content was assessed. Representative trichrome images of the wounds at lower (top row) and high magnification (bottom row) of the area enclosed in boxes are shown (FIG. 12A). Collagen content is quantified per HPF using color thresholding in ImageJ (FIG. 12B). Photographs of wounds at 28 days after surgery. For scar assessment, photographs were taken from all 4 treatment groups before harvest (FIG. 12C). Scale bar, 50 μm. 3 $ n $6, bar plots indicate mean±SD with of values per each mouse wound indicated. Details of compositions of UNIX, TLS, CPOc and CPO are in Table 3.

FIG. 13 shows photographs of wounds after surgery. Lignin composites were crosslinked in situ and wounds were monitored in wildtype C57BU6N mice (8 to 10-week-old) up to 28 days. Photographs were taken at 1, 3 and 7 days after surgery. Details of compositions of UNTX, TLS, CPOc and CPO are in Table 3.

FIG. 14 shows dissolved oxygen measured in GelMA and TLS composites over 7 days. Since TLS confers additional crosslinking to GelMA via thiol-ene chemistry, the release of trapped oxygen is delayed in comparison to GelMA matrix. However, both composites reached the base level around 6.3 ppm over 7 days. Details of compositions of TLS are in Table 3.

FIGS. 15A-15B show dissociation of peroxide from CaO₂. (FIG. 15A) The pH of PBS altered the released amount of peroxide from CaO₂. (FIG. 15B) The solubility limit of CaO₂ is 1.65 mg/mL. When CaO₂ was dissolved at PBS (pH 7.4, 37° C.), the dissociation of peroxide to oxygen was catalyzed by catalase (100 U/mL). While neither SLS nor TLS significantly catalyzed the dissociation of peroxide to oxygen at 50 μg/mL of CaO₂, TLS facilitates the dissociation of peroxide to oxygen at 5 μg/mL of CaO₂. First symbol:+, CaO₂ 50 μg/ml;−, CaO₂ 5 μg/ml/Second symbol: +, catalase 100 U/mL;−, catalase 0 U/mL/Third symbol:+, SLS or TLS 3 mg/mL;−, SLS or TLS 0 mg/mL. Student's t-test, ** p<0.01; n=3, mean±SD.

FIGS. 16A-16B show minor reduction of antioxidation capacity of SLS and TLS in the presence of CaO₂. (FIG. 16A) DPPH (2,2-diphenyl-1-picrylhydrazyl) inhibition in the absence of CaO₂, similar to previously reported results. (FIG. 16B) DPPH inhibition in the presence of CaO₂ at concentrations ranging from 5 to 50 μg/mL. One-way ANOVA with Tukey's post hoc test, *p<0.05 and ** p<0.01, ^{#}denotes the exception to the comparison of L-asc to TLS. N=3, mean±SD.

FIG. 17 shows intracellular ROS of C2C12 myocytes was measured by adding DCFDA (dichlorodihydrofluorescein diacetate) and fluorescence microscopy. C2C12 myocytes were treated with H₂O₂ for 2 h, followed by TLS treatment (at 0.1 and 1.0 mg/mL) for 18 h. Scale bar, 200 μm.

FIGS. 18A-18B show quantification of aSMA+ cells in the wound treated with lignin composites. aSMA+ cells quantified in the edge sections (FIG. 18A) and middle sections (FIG. 18B) of the wound (total shown in FIGS. 10A-10E). Either one-way ANOVA with Tukey's HSD post hoc tests or with Kruskal-Wallis test followed by Dunn's test shows no statistically significant difference. 3≤n≤6, mean±SD. Details of compositions of UNTX, TLS, CPOc and CPO are in Table 3.

FIGS. 19A-19B show fibrosis PCR array of human dermal fibroblasts (hdFBs). (FIG. 19A) Principal Component Analysis (PCA) of 84 fibrosis-related genes from high scar (HS) and low scar (LS) hdFBs cultured on three different substrates (TCPS, GelMA and TLS, see Table 3). (FIG. 19B) Hierarchical Clustering (HC) of 84 fibrosis-related gene. LS exhibits a proliferative phenotype. TLS lignin composites modulate the profiles of the fibrosis-related genes.

FIG. 20 shows representative results of Angiogenesis Analyzer for the micrographs taken after 96 h of MVEC (microvascular endothelial cell) culture initiation. GelMA (50 mg/mL); TLS—GelMA 50 mg/mL with TLS 3 mg/mL; CPOc-GelMA 50 mg/mL with TLS 3 mg/mL and CPO nanoparticle 4 mg/mL without incorporating CaO₂; CPO-GelMA 50 mg/mL with TLS 3 mg/ml and CPO nanoparticle 4 mg/mL.

FIG. 21 shows seven angiogenic features were analyzed to assess the performance of lignin composites in two different conditions (normal 5.5 mM glucose in the culture medium; orange, high glucose 30 mM glucose in the culture medium): number of extremities, number of nodes, number of junctions, number of branches, number of isolated segments, sum of isolated branch lengths, and average mesh sizes. Mean±SD, n=6-9 independent samples, 3 images/composite were analyzed. p values by one way ANOVA and Tukey's post hoc tests.

FIGS. 22A-22B show analysis of levels of HIF-1α; ELISA quantification of HIF1α (hypoxia-inducible factor-1a) at 96 h (FIG. 22A) and VEGF (vascular endothelial growth factor) at 24 h (FIG. 22B) from supernatants of MVECs. Mean±SD, n=3. Student's t-test within the same group

FIG. 23A shows healing progression of db/db mouse wounds treated with lignin composite TLS, CPO, or CPOc at 7 days post-wounding compared to UNTX wounds. FIG. 23B shows gross images taken at indicated intervals post-wounding, with representative H&E staining of post-wounding day 7 wounds, Scale bar=50 μm. FIGS. 23C-23D show quantification of epithelial gap (FIG. 23C) and granulation tissue area (FIG. 23D) at day 7 post wounding. Mean±SD, n=3 wounds per treatment group, *p<0.05 by one way ANOVA and Tukey's post hoc tests.

FIGS. 24A-24F show neovascularization panel in db/db mouse wounds treated with lignin composite TLS, CPOc, or CPO immediately post-wounding compared to UNTX wounds. Staining of day 7 wound sections with antibodies against CD31 (FIG. 24A), VEGF (FIG. 24B), and HIF-1α (FIG. 24C), Scale bar=50 μm. (FIG. 24D) CD31 staining of wound sections showed a significant increase in lumen density per HPF in CPO wounds at day 7. Day 7 wound tissue homogenates evaluated using ELISA (enzymeOlinked immunosorbent assay) revealed an increasing trend of VEGF expression (FIG. 24E) and significantly decreased HIF-1α expression (FIG. 24F) in CPO wounds. Mean±SD, n=3 wounds per treatment, *p<0.05 and ** p<0.01 by one way ANOVA and Tukey's post hoc tests.

FIGS. 25A-25D show inflammatory panel in db/db mouse wounds treated with lignin composite TLS, CPOc, or CPO immediately post-wounding. Representative images of stained day 7 wound sections with antibodies against CD45 (FIG. 25A) and F4/80 (FIG. 25C). Scale bar=250 μm. Quantification of % of CD45+(FIG. 25B) and F4/80+(FIG. 25D) cells in HPF in the wound sections showed no significant increase in inflammatory markers in CPOc or CPO wounds at day 7. Mean±SD, n=3 wounds per treatment, *p<0.05 by one way ANOVA and Tukey's post hoc tests.

FIGS. 26A-26C show Healing progression of db/db mouse wounds treated with lignin composite TLS, CPOc, or CPO immediately post-wounding. At 14 days post-wounding as compared to UNTX wounds. (FIG. 26A) Representative gross images taken at time of tissue collection at day 14 post-wounding shows improved healing in CPOc and CPO wounds, with representative H&E staining of the wound sections. Scale bar=50 μm. (FIG. 26B) Staining of day 14 wound sections with antibodies against CD31. Scale bar=50 μm. (FIG. 26C) Quantification of CD31+ lumens showed an increase in lumens in the CPO lignin composite treated group at day 14. Mean±SD, n=2 wounds per treatment.

FIG. 27 shows a reaction scheme for a Mannich reaction to confer aminoalkyl groups to TLS.

FIG. 28 shows preliminary ¹H NMR results for the reaction shown in FIG. 27, demonstrating the changes at 0=2.8-3.0 ppm and 0=1.1-1.9.

FIG. 29 shows representative dual staining with anti-CD206 and anti-arginase1 antibodies. Increased double positive (M2-type macrophages) in db/db mouse wounds treated with CPO composites. Dotted line depicts the boundary of the granulation tissue and the underlying connective tissue.

FIG. 30 shows VEGF levels were measured in db/db mouse wounds treated with composites with ProteinSimple WES on homogenized wound tissues from day 7 post-wounding. n=3 wounds per treatment group.

FIGS. 31A-31B show results of bulk RNA sequencing of MVECs cultured on lignosulfonate composites cultured under Normal (5.5 mM glucose) and high Glucose (30 mM glucose) media. (FIG. 31A) PCA of the groups. (FIG. 31B) Unsupervised DGE and heatmaps showing top 100 differentially expressed genes among ECs on lignosulfonate composites. n=2-3 independent samples per groups were analyzed.

FIGS. 32A-32B show results of bulk RNA sequencing of MVECs cultured on CPO and GelMA-Glucose control lignosulfonate composites under high glucose (30 mM glucose) media conditions. (FIG. 32A) The volcano plot shows significantly up/down-regulated genes relevant to HIF, angiogenesis and vascular maturation. (FIG. 32B) GO enrichment analysis (BP) also highlights responses to hypoxia, regulation of vasculature development and cellular response to hypoxia.

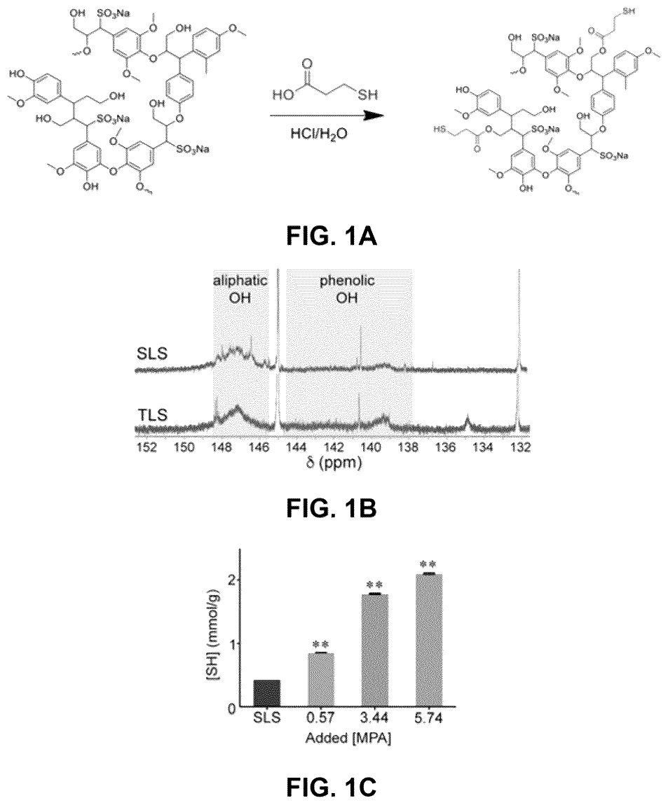

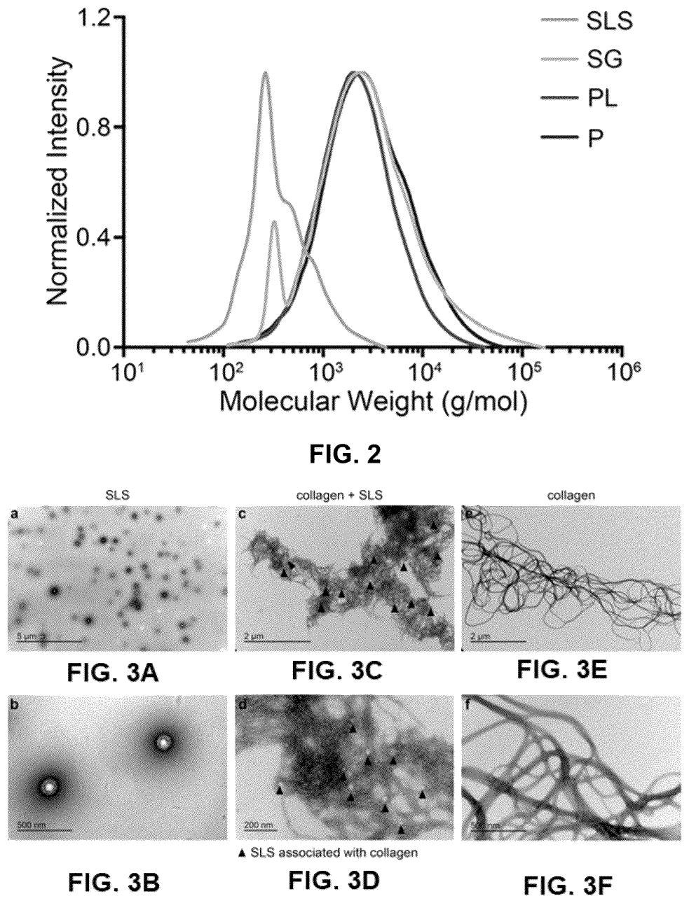

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, bioavailability of orally-delivered antioxidants and lignin compositions can be relatively low, and local/regional delivery to wounds may better promote wound healing. Therefore, in one aspect, provided herein are injectable lignin composites with the following components and properties: 1) lignosulfonate with thiolation (TLS) that scavenges ROS from wounds, 2) lignosulfonate (SLS) which encapsulates CaO₂ while scavenging radicals from CaO₂ and simultaneously protecting CaO₂ from the aqueous environment of tissue or hydrogel, and 3) GelMA that modulates the mechanical properties of lignin composites to support injectability and/or aerosolization. In an aspect, first two rationales can confer dual functionality to lignosulfonate.

Also disclosed herein is an integrated system of CaO₂ and SLS-PLGA (poly(lactic-co-glycolic) acid) nanoparticles (NPs) coupled with the release of O₂ from lignin composites. In one aspect, and without wishing to be bound by theory, a core-shell structure of SLS-PLGA NPs can be used to simultaneously deliver CaO₂ and to protect CaO₂ from aqueous microenvironments while NPs, after depleting CaO₂, still serve as a ROS scavenger. Further in this aspect, swelling/degradation profiles and mechanical properties of lignin composites can be assessed. In still another aspect, the disclosed lignin composites, when applied to or in the wounds of mammals, including but not limited to, wild-type (WT) mice can be shown to enhance wound healing responses including tissue granulation, neovascularization, inflammatory responses, and scarless/regenerative outcomes.

In an aspect, the disclosed system not only scavenges reactive oxygen species but also produces oxygen without increasing detrimental byproducts (i.e. H₂O₂), to serve as a correcting, provisional matrix for wounds, including, but not limited to, diabetic wounds.

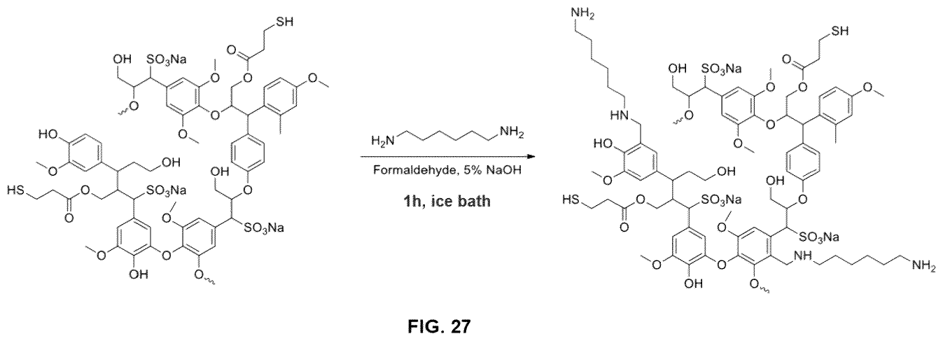

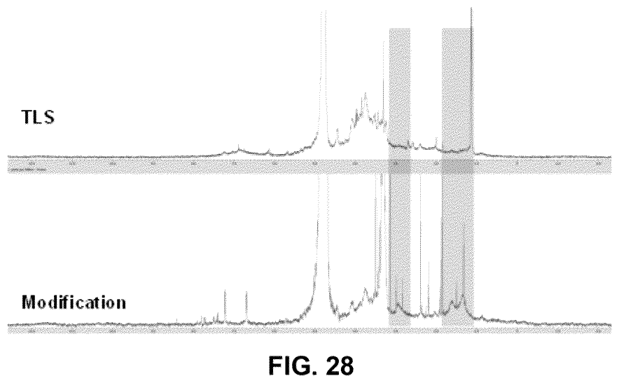

In one aspect, disclosed herein is a composition including (a) one or more lignin derivatives, (b) at least one inorganic peroxide, and (c) a pharmaceutically acceptable carrier. In a further aspect, the one or more lignin derivatives can be or include TLS, SLS, aminoalkyl TLS (FIG. 27) or any combination thereof. In one aspect, the amount of sulfonate in lignosulfonate ranges from about 0.7 to about 2.5 mmol/g. In a further aspect, and without wishing to be bound by theory, since the thiolation and conjugation of PLGA to SLS as disclosed herein uses the hydroxyl groups of SLS, the sulfonate quantity is not altered. In some aspects, however, the sulfonate can be protonated and may be useful as an acid catalyst for the disclosed esterification. In one aspect, the sulfonate itself is stable during the disclosed reactions. In some aspects, amino group-containing substituents can be added to the TLS to confer antibacterial properties. Further in this aspect, the antibacterial properties can treat or prevent infections in wounds, thereby assisting with healing and/or improving the wound-healing properties of the disclosed compounds and compositions.

In one aspect, the TLS can include from 0.5 to about 6 mmol of thiol groups per gram of TLS, or from about 0.7 to about 2.1 mmol of thiol groups per gram of TLS. In another aspect, the pharmaceutically-acceptable carrier can be a hydrogel and can be formed from GelMA or another hydrogel forming polymer. Additional polymers include, but are not limited to, other acrylate-functionalized ibopolymers, synthetic polymers, and peptides, including other ECM-derived proteins and proteoglycans such as, for example, collagen and hyaluronan. Semisynthetic polymers including GelMA mixed with poly(ethylene glycol) diacrylate (PEGDA) at a ratio about 20:80 can also be used for cell attachment, but if cell attachment is not required, GelMA and PEGDA can be used at other ratios such as, for example, from about 90:10 to about 10:90. Without wishing to be bound by theory, the GelMA or other hydrogel forming polymer may modulate properties of the lignin derivative such that the lignin derivative is suitable for administration by injection, or can be administered by aerosolization or droplet-like formulation. In one aspect, the composition can be administered once, or can be administered once on initial presentation and at least one time every other day for up to 14 days. In some aspects, droplet and/or aerosolized compositions may not form a hydrogel; however, in other aspects, well-crosslinked polymers can be used. In any of these aspects, the at least one inorganic peroxide can be CaO₂, MgO₂, or any combination thereof. In some aspects, the inorganic peroxide is encapsulated in the core-shell nanoparticle, wherein the shell can be a functionalized lignin grafted to one or more polymers or copolymers. Further in this aspect, the functionalized lignin can be SLS and the one or more polymers or copolymers can be poly(lactic-co-glycolic) acid (PLGA), polycaprolactone, or any combination thereof.

Also disclosed herein is a method for treating a wound in a subject, the method including administering a disclosed composition to the subject. In one aspect, the composition can be administered topically, by injection, via aerosol or droplets, or any combination thereof. In a further aspect, the subject can be a mammal such as, for example, a human, cat, dog, rat, mouse, rabbit, hamster, guinea pig, or pig. Without wishing to be bound by theory, pigs are especially useful model organisms for studying wound healing applications, and can include Yorkshire domestic pigs, red Duroc pigs, and other pigs. In any of these aspects, the subject can have diabetes, hypertension, obesity, or another metabolic condition, or can have a polymicrobial infection, systemic inflammation, or any combination thereof or other metabolic syndromes including but not limited to hypertension and/or obesity, and polymicrobial infections and systemic inflammation.

In a further aspect, performing the method can enhance at least one wound healing response relative to an untreated wound, wherein the at least one wound healing response can be increased tissue granulation, increased neovascularization, reduced inflammatory response, reduced scarring, or any combination thereof. In still another aspect, the shell of the core-shell nanoparticle can continue to scavenge ROS even after depletion of the inorganic peroxide.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a functionalized lignin,” “a pharmaceutically acceptable carrier,” or “an antioxidant,” include, but are not limited to, mixtures or combinations of two or more such functionalized lignins, pharmaceutically acceptable carriers, or antioxidants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an antioxidant refers to an amount that is sufficient to achieve the desired improvement in the property modulated by the formulation component, e.g. achieving the desired level of control of ROS. The specific level in terms of wt % in a composition required as an effective amount will depend upon a variety of factors including the amount and type of ROS; extent of wound including size, presence of infection, and patient comorbidities; amount and type of functionalized lignins, and desired sustained release profile for the antioxidant.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Alkene Incorporation in Gelatin

### Methods

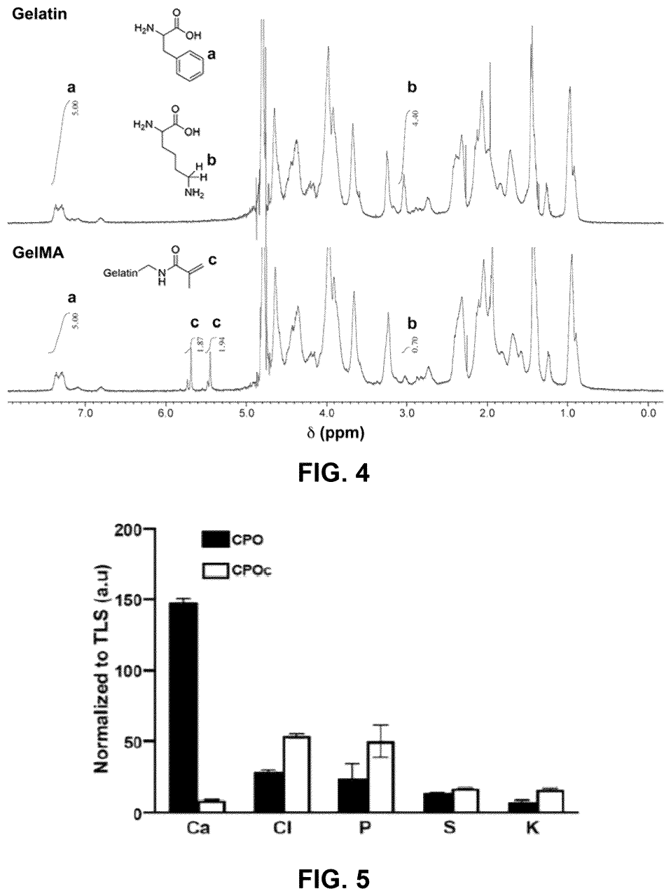

Alkene was incorporated into gelatin by the coupling reaction of gelatin with methacrylate acid (MA). Gelatin (1.00 g) was dissolved in 10.0 mL of PBS (pH=7.4) at 50° C. Separately, a solution of MA (0.10 g), N-ethyl-N′-(3-(dimethylamino)propyl)carbodiimide (EDC; 0.178 g), N-hydroxysuccinimide (NHS, 0.107 g) and dimethyl sulfoxide (DMSO, 2.00 mL) was prepared by stirring at 40° C. for 30 min. The flask containing the gelatin solution was placed in an oil bath at 50° C., and the other solution of MA, EDC, NHS and DMSO was added dropwise. The mixture was further stirred at 50° C. for 1.5 h. The solution was cooled down to room temperature, followed by dialysis in water at 40° C. for a week. The resulting solution was lyophilized to yield alkene-incorporated gelatin powder sample. The coupling efficiency of MA to gelatin was assessed using 1H NMR comparing the phenyl peak (a) and the lysine peak (b) (FIG. 4). Upon coupling reaction, the development of two distinct peaks between 5 to 6 ppm were observed in ¹H NMR spectrum, referring to the two protons in alkene group of MA attached to the lysine of gelatin. Additionally, the gelatin phenylalanine (Phe) group remained untouched during the reaction and allowed determination of the mmol/g ratio of the GelMA. From literature, native porcine gelatin contains 15.5 mol/105 g of Phe. Combining this information with the ratio of Phe:MA, the mmol/g concentration of MA was calculated using the following equation: MA concentration (mmol/g)=(15.5 mol/105 g)×(1000 mmol/mol)×(Iphe/lakene) (intensity ratio of phenyl to alkene). The batch presented in FIG. 4 showed the phe/lalkene of 1:1.905 ([intensity from five protons of Phe/5]/[intensity of two protons of alkene/2]) resulting in a MA concentration of 0.295 mmol/g. This value also can be utilized to estimate the degree of substitution with a ratio of 1:2.20 ([intensity from five protons of Phe/5]/[intensity of two protons on carbon adjacent to NH₂ (c) in Lys/2]) of Phe:Lys of the pristine gelatin (i.e. [reacted MA upon the substitution]/[reactive amine site (Lys) in pristine gelatin]×100=1.905/2.20×100=87%), suggesting that the modifications made in this synthesis route produced GelMA with around 87% substitution.

In one experiment, the concentration of methacrylate in GelMA was 0.295 mmol/g, and the extent of reaction was 0.87 (i.e. 87% of lysine groups reacted with methacrylic acid).

### Results

Results for GPC of alkali-extracted lignins and SLS are presented in Table 1:

TABLE 1
GPC of Alkali-Extracted Lignins and SLS

| | M̅_{w} | M̅ₙ | PDI_{polymer} |
|---|---|---|---|
| lignin | (g/mol) | (g/mol) | (M̅_{w}/M̅ₙ) |
| poplar (PL) | 3100 | 1900 | 1.63 |
| pine (P) | 4500 | 1800 | 2.50 |
| switchgrass (SG) | 5400 | 1400 | 3.86 |
| SLS | 460 | 280 | 1.64 |

Dynamic Light Scattering (DLS) results for nanoparticles are presented in Table 2:

TABLE 2
DLS results of nanoparticles

| | | PdI_{DLS} |
|---|---|---|
| Nanoparticles | Average dia. ± st. dev. (nm) | (st. dev./mean dia.) |
| SLS | 126.6 ± 7.2 | 0.585 ± 0.017 |
| SLS-PLGA | 171.6 ± 1.9 | 0.204 ± 0.025 |
| (w/o CaO₂) | | |
| SLS-PLGA/ | 140.8 ± 0.5 | 0.0597 ± 0.0199 |
| CaO₂ | | |
| TLS | 58.01 ± 13.54 | 0.258 ± 0.056 |
| TLS in TCEP | 7.728 ± 0.090 | 0.442 ± 0.005 |

dia: diameter
st.dev.: standard deviation

### Example 2: Injectable Antioxidant and Oxygen-Releasing Lignin Composites to Promote Wound Healing

### Materials and Methods

Synthesis of NPs: The coupling of lignosulfonate to PLGA (poly(lactic-co-glycolic) acid) was performed by acylation reaction for SLS in a mass ratio of 2 to 1. The final precipitate was collected and dried for 2 days under vacuum. Then, SLS-graft-PLGA (SLS-PLGA) was dissolved in ethyl acetate (EtOAc, organic phase) and calcium peroxide (CaO₂, Cat #466271, Sigma-Aldrich, St. Louis, MO, USA) and dissolved under stirring at room temperature. The organic phase was added to the aqueous phase (low resistivity water) under stirring. The suspension was homogenized with a microfluidizer (Microfluidics Corp., Westwood, MA, USA) at 30 kpsi at 4° C. Next, the organic solvent was evaporated with a rotary evaporator R-300 (Buchi Corp, New Castle, DE, USA). Finally, trehalose was added (1:1 mass ratio) as a cryoprotectant for the lyophilization of SLS-PLGA NPs and stored at −20° C. for further characterization and experiments.

DLS of NPs: NPs were synthesized with the previously published methods. The analysis of SLS-PLGA showed a spherical, core-shell structure with a relatively smooth surface as evidenced by small-angle scattering data and transmission electron micrographs. Size, polydispersity and Z-potential of NPs of CPO and TLS (0.2-0.4 mg/mL) were measured by dynamic light scattering (DLS) using Malvern Zetasizer ZS (Malvern Panalytical, Westborough, MA, USA). NP suspensions were filtered through a 0.45 μm filter. To prevent the formation of disulfide bonds, TLS sample was prepared in 250 mM Tris(2-carboxyethyl)phosphine hydrochloride (TCEP HCl, Cat #H51864, Alfa Aesar, Haverhill, MA, USA) in PBS.

Elemental analysis of NPs: The nuclear microscopy setup at the Louisiana Accelerator Center was used to probe the concentrations of calcium in these samples using particle-induced x-ray emission (PIXE) spectrometry. The samples were placed in a low-pressure environment (≤1×10⁻⁶ mbar) on electrically conductive non-porous carbon tape attached to the sample holder. A focused (10 μm×10 μm) 2 MeV proton beam, with a beam current in the range of 10-20 pA, raster scanned the sample region (1 mm×1 mm) for about 1 h. A silicon drift detector was placed at 135° in front of the sample to detect the characteristic x-rays excited by energetic protons. Analysis of the spectra was performed with the GeoPIXE™ software (v7.3). The elemental maps and the derived concentrations were generated by the dynamic analysis method using average matrix composition from the whole scanned area.

Formation of lignin composites: TLS was synthesized with the previously published protocol. GelMA was synthesized by the coupling reaction of gelatin with methacrylic acid. The alkene incorporation to gelatin was confirmed with ¹H NMR (FIG. 4). Lignin composites were formed by weighing out GelMA, lithium phenyl-2,4,6-trimethylbenzoylphosphinate (LAP, Allevi, Philadelphia, PA, USA) and NPs of TLS, SLS-PLGA/CaO₂ or SLS-PLGA (w/o CaO₂) and by mixing GelMA with LAP and NPs in PBS at 37° C. as summarized in Table 3. Any concentrations of TLS higher than 7 mg/mL interfered with photo-crosslinking of lignin composites, leading to the reduction of elasticity of lignin composites with partially crosslinked lignin composites. The concentrations of GelMA, LAP, and TLS were fixed at 50, 5, and 3 mg/mL, respectively, and the concentrations of SLS-PLGA/CaO₂ or SLS-PLGA (w/o CaO₂) were varied at either 4 or 40 mg/mL. Each precursor was pipetted into a custom PDMS mold (8 mm diameter and 1 mm height, 50 μL) to form lignin composites. Samples were crosslinked using a UV floodlamp (Intelli-Ray 400, Uvitron international, West Springfield, MA, USA) for 30 sat 10 mW/cm².

TABLE 3
Abbreviations for Lignin Composites

| Abbreviation | Matrix | Nanoparticles (NPs) |
|---|---|---|
| UNTX | None | None |
| GelMA | GelMA | None |
| TLS | GelMA | TLS |
| CPOc | GelMA | TLS and SLS-PLGA (w/o CaO₂ |
| CPO | GelMA | TLS and SLS-PLGA/CaO₂ |

Quantification of O₂ release from lignin composites: The released O₂ was optically measured by a planar O₂ sensor spot (SP-PSt3-SA23-D5-OIW-US, PreSens, Regensburg, Germany) placed at the bottom of a 96-well plate. Lignin composites (5 mm diameter and 1 mm height, 20 μL) were placed on top of the planar O₂ sensor spot and submerged in serum-free medium (37° C./5% CO2). To prevent evaporation, each well was covered with a sealing film, and the entire plate was wrapped with a sealing tape. A polymer optical fiber (POF-L2.5-2SMA, PreSens, Regensburg, Germany) optically sensed the concentration of O₂ through the bottom of a well and sent signals to an O₂ sensor (OXY-1 SMA, PreSens, Regensburg, Germany) for measurement.

Swelling ratio and in vitro degradation of lignin composites: Swelling ratios of lignin composites were determined by the following equation: [Wₛ−W_{d}/W_{d}, where Wₛ and W_{d} represent the weight after swelling in PBS for 24 h and the weight after lyophilization, respectively. In vitro degradation of lignin composites was determined by submerging lignin composites in the solution of collagenase type II (0.5 U/mL, cat #CLS-2, Worthington Biochemical) with 1 mM CaCl₂) in serum-free culture medium, along with a control group without including the collagenase. Lignin composites were collected at 0, 2, 6 and 24 h and lyophilized to determine the fraction of remaining composites.

Oscillating rheometry of lignin composites: Using a TA Discovery HR-2 rheometer and the previously published methods, viscosity of the precursors of lignin composites were measured in a flow ramp setting (shear rate from 1 to 2000 (1/s)) and with a 25 mm parallel plate. Using an 8 mm parallel plate, storage (G′) and loss (G″) moduli of lignin composites were determined by frequency sweeping from 0.62 to 19.9 (rad/s) at 2% strain. Since storage moduli are altered by axial stress applied during measurement, the slope of axial stress vs compression was evaluated, similar to evaluating Young's modulus from the slope of a stress-strain curve. Axial stresses at 0, 10, and 20% of compression were determined, while lignin composites were subject to 2% strain and 6.28 rad/s frequency.

Animal model of wound healing: Wound healing studies were carried out in WT C57BL/6N mice (8- to 10-week-old, female and male). Mice were maintained under pathogen-free conditions with access to food and water ad libitum in the Texas Children's Hospital Feigin Center animal facility. Protocols for animal use were approved by the Institutional Animal Care and Use Committee at Baylor College of Medicine (#AN-6880). At the time of wounding, mice were anesthetized using isoflurane, the backs were shaved, and prepped for surgery with three times alternating betadine and 70% isopropyl alcohol scrubbing. Two 6 mm diameter full thickness wounds were made using a 6 mm dermal punch biopsy, excising tissue through the panniculus carnosus muscle. Skin contraction was controlled through application of a silicone stent with inner diameter of 8 mm and outer diameter of 16 mm, secured concentric to the wound using skin adhesive and 6 simple interrupted 6-0 Proline® sutures (Ethicon, Raritan, NJ). Wounds were maintained in a moist wound environment using a semi-occlusive sterile adhesive dressing (Tegaderm, 3M, St. Paul, MN, USA) and controlling the skin contraction through application of the stent permit wounds to heal in a humanized pattern via granulation tissue deposition and re-epithelialization. Prior to dressing with Tegaderm, each wound received one of the four treatments: a standard saline wash (untreated, UNIX), TLS, CPOc and CPO (summarized in Table 3). Crosslinking of the treatments was performed immediately after application to the wound bed using a UV floodlamp (B-1 00AP High Intensity, Blak-Ray) for an exposure time of 30 s. Wounds were imaged at 1, 3, and 7 days post operatively (FIG. 13) and harvested at days 7 and 28. For histology and immunostaining, wounds were bisected in the rostral-caudal plane, fixed overnight in 10% neutral buffered formalin, dehydrated through a series of graded ethanol and xylene, and embedded in paraffin wax. Five-micrometer-thick sections from the paraffin-embedded wounds were collected using a RM 2155 microtome (Leica, Heidelberg, Germany) and used in staining.

Morphometric quantification: At day 7, epithelial gap and granulation tissue areas were measured from H&E (Cat #3801571 and Cat #380161 5, respectively; Leica, Heidelberg, Germany) stained sections using morphometric image analysis (LASX, Leica, Heidelberg, Germany). Staining was carried out on 5 μm formalin fixed paraffin embedded (FFPE) sections following deparaffinization and rehydration in xylene and graded ethanol as following the manufacturer's recommendations. Epithelial gap was determined using a full, 4× tile scan of the wound bed, and measuring the distance (in mm) between the leading epithelial margins on either side of the wound cross-section. Granulation tissue area was determined using a standardized approach of calculating the entire wound area (in mm²) above the panniculus carnosus and bounded within the wound edges laterally. If parts of the hydrogel remained devoid of infiltrated cells, that area was excluded from the granulation tissue assessment.

Immunostaining: Immunohistochemistry was carried out on serial 5 μm sections from day 7 FFPE tissues, which were deparaffinized and rehydrated to water using xylene and graded ethanol. Primary antibodies used were aSMA (rabbit anti-mouse, Cat #ab5694, Abeam, Cambridge, United Kingdom, 1:500 dilution) to measure myofibroblast infiltration, CD31 (rabbit anti-mouse, Cat #ab28364, Abeam, Cambridge, United Kingdom, 1:100 dilution) to measure endothelial cells and vascular lumen density, CD45 (rabbit anti-mouse, Cat #ab10558, Abeam, Cambridge, United Kingdom, 1:5000 dilution) to determine pan-leukocyte infiltration, CD206 (rabbit anti-mouse, Cat #ab64693, Abeam, Cambridge, United Kingdom, 1:500 dilution) to measure M2 macrophage levels, F4/80 (rat anti-mouse, Cat #MF48000, Thermo Fisher Scientific, Waltham, MA, USA, I:5000 dilution) to measure pan macrophage infiltration, and Ly6G (rat anti-mouse, Cat #551459, BD Biosciences, Franklin Lakes, NJ, USA, 1:5000 dilution) to determine the infiltration of monocytes, granulocytes, and neutrophils.

Following deparaffinization and rehydration to water, sections were then immersed in target antigen retrieval solution (Cat #K800521-2, Agilent, Santa Clara, CA, USA) and treated following the protocol within the DAKO PT Link Rinse Station (Cat #PT10930, Leica, Heidelberg, Germany). Following antigen retrieval, wound sections were stained using the DAKO autostainer (AS480, Leica, Heidelberg, Germany). First, the sections were buffered in DAKO wash buffer (Cat #K800721-2, Agilent, Santa Clara, CA, USA) before incubating in primary antibody diluted in DAKO antibody diluent (Cat #S080983-2, Agilent, Santa Clara, CA, USA) for 1 h. The primary antibody was then rinsed, and the sections were washed in wash buffer before incubation in the appropriate the secondary antibody system, either HRP (horseradish peroxidase)-Rabbit (Cat #K400311-2, Agilent, Santa Clara, CA, USA) or HRP-Rat (Cat #D35-110, GBI Labs, Bothell, WA) for 20 min. The secondary antibody was then rinsed off using wash buffer, and the appropriate visualization system was applied-DAKO DAB (Cat #K346811-2, Leica, Heidelberg, Germany) for all but CD206 which utilized AEC (Cat #K400511-2, Leica, Heidelberg, Germany). A hematoxylin (Cat #K800821-2, Agilent, Santa Clara, CA, USA) counterstain was then applied, and sections were dehydrated in xylene and graded ethanol before putting coverslips were applied using a xylene based mounting media (Cat #23-245691, Thermo Fisher Scientific, Waltham, MA, USA)-except for those sections using AEC. AEC visualization system which required an aqueous mounting media (Cat #108562, Merck KGaA, Darmstadt, Germany).

Staining was quantified using images taken on the Leica DMI8 camera. For all data, the percentage of positive cells were determined by counting the number of positive cells per HPF (40× magnification) and divided by the total number of cells in that HPF as determined by the hematoxylin counterstain. Final values were determined by the average of 4 to 6 HPFs per wound. The total numbers of vessel lumens were counted per HPF. These percent values or vessel counts were then averaged from 6 images taken across the wound bed to determine the final value.

Scar assessment: Serial 5 μm sections from day 28 FFPE wound sections were deparaffinized and rehydrated to water using xylene and graded ethanol. Sections were then stained using Gomori's Trichrome as following the manufacturer's recommendations (Cat #38016SS2, Leica, Heidelberg, Germany). Collagen content per HPF in the dermis of the scars was measured using established methods with color-thresholding in ImageJ in which color segmentation was used to isolate only pixels representing collagen fibers, thus allowing quantification of the amount of collagen within the selected area. Final values were determined by the average of 4 to 6 HPFs per wound. Gross images of the wound at day 28 were also obtained, and a subjective assessment of scarring was performed.

Statistical analysis: For multiple comparisons, one-way ANOVA with Tukey's post hoc tests or with Kruskal-Wallis test followed by Dunn's test was performed, where p values <0.05 or <0.01 were considered significant. At least three independent experiments were performed. Four to five mice were included per treatment per each time point.

### Results and Discussion

The incorporation of CaO₂ in NPs of SLS-PLGA led to a narrower distribution of NP diameters: The present effort to apply the antioxidant capability of TLS to wound microenvironments would be synergistic with other pro-regenerative stimuli. It was hypothesized that controlled release of oxygen while scavenging ROS by TLS in the wound microenvironments will promote wound healing. Thus, CaO₂ was incorporated in NPs of SLS-PLGA/CaO₂ and found that the size of NPs is not significantly altered, even when compared to NPs of SLS-PLGA (w/o CaO₂), as shown in Table 4. In comparison to SLS, the average diameter of NPs of SLS-PLGA/CaO₂ increased while the Pdl of NPs of SLS-PLGA/CaO₂ was significantly smaller than that of NPs of SLS-PLGA (w/o CaO₂). SLS is a biomaterial with potential lot-to-lot variability. However, the synthesis of NP with CaO₂ slightly increased the average diameter and significantly reduced Pdlpus upon the incorporation of CaO₂. In contrast, NPs of SLS-PLGA (w/o CaO₂) were formed only with ethyl acetate, forming irregular shapes possibly with cavity. The average diameter of TLS is significantly smaller than SLS or NPs of SLS-PLGA. After adding TCEP to cleave possible disulfide bonds in TLS, the average diameter of TLS was further reduced. As evidenced in Table 4, TLS formed NPs with a broader distribution. The formation of NPs of SLS-PLGA requires stirring at room temperature, while the thiolation of SLS is completed via acid-catalyzed esterification at 80° C. In PBS (pH 7.4), NPs were formed via interparticle disulfide formation with thiols in TLS, but adding TCEP to PBS dissociates NPs of TLS into smaller NPs. This could be an advantage to form lignin composites with homogeneous distribution of TLS in the matrix of GelMA. After the degradation of GelMA, the reduced form of TLS can be cleared by glomerular filtration (5-10 nm).

Due to the size (diameter) of NPs, the incorporation of CaO₂ was assessed by PIXE spectrometry. NPs of SLS-PLGA with or without CaO₂ showed similar composition of elements while the normalized concentration of Ca in NPs of SLS-PLGA/CaO₂ is about 17 times higher than that of NPs of SLS-PLGA (w/o CaO₂) in the quantitative molecular spectroscopy (FIG. 5).

TABLE 4
Dynamic Light Scattering Results of Nanoparticles

| | | PdI_{DLS} |
|---|---|---|
| | Average | (standard |
| | diameter ± | deviation/ |
| | standard | mean |
| Nanoparticles | deviation (nm) | diameter) |
| SLS | 126.6 ± 7.2 | 0.585 ± 0.017 |
| SLS-PLGA (without CaO₂) | 171.6 ± 1.9 | 0.204 ± 0.025 |
| SLS-PLGA/CaO₂ | 140.8 ± 0.5 | 0.0597 ± 0.0199 |
| TLS | 58.01 ± 13.54 | 0.258 ± 0.056 |
| TLS in TCEP | 7.728 ± 0.090 | 0.442 ± 0.005 |

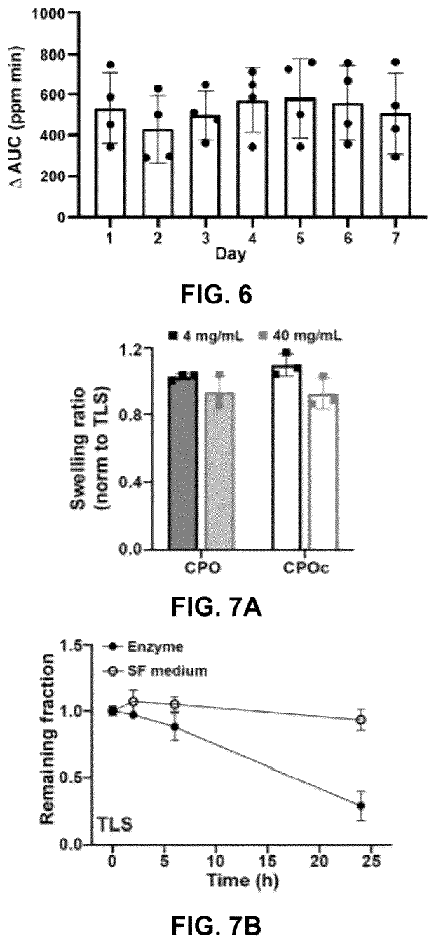

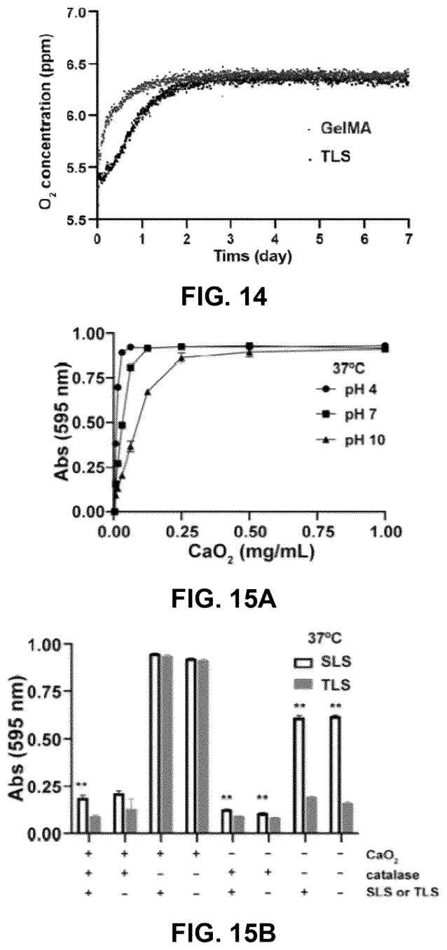

Oxygen released from CPO lignin composites was maintained at around 700 ppm/day from each composite for 7 days: To measure the released quantity of O₂ from lignin composites, a planar O₂ sensor was used with optical measurement. These planar sensors can measure the concentration of O₂ in liquid or gas. The difference (A) of the AUC between lignin composites with NPs of SLS-PLGA/CaO₂ and of SLS-PLGA (w/o CaO₂) was calculated over 1440 min each day. It was also confirmed that the base level concentration of O₂ from GelMA matrix or TLS composite was conformed to that of SLS-PLGA (w/o CaO₂) around 6.3 ppm (FIG. 14). As shown in FIG. 6, the difference is around 500 ppm (0.05% O₂) per day from the lignin composite with 5 mm diameter and 1 mm height (20 μL). This amount can be scaled to 700 ppm per day with lignin composites (6 mm diameter and 1 mm height) for the animal experiments. As the statistical difference is not detected, the oxygen release is maintained up to day 7, which is also distinguished from other methods of O₂ delivery by CaO₂. It was observed that the swelling of lignin composites over the first 24 h contributed to the slightly higher AAUC in day 1 than that in day 2 since lignin composites were placed in a well of the 96-well plate with the planar O₂ sensor and serum-free medium was added without achieving equilibrium swelling of lignin composites.

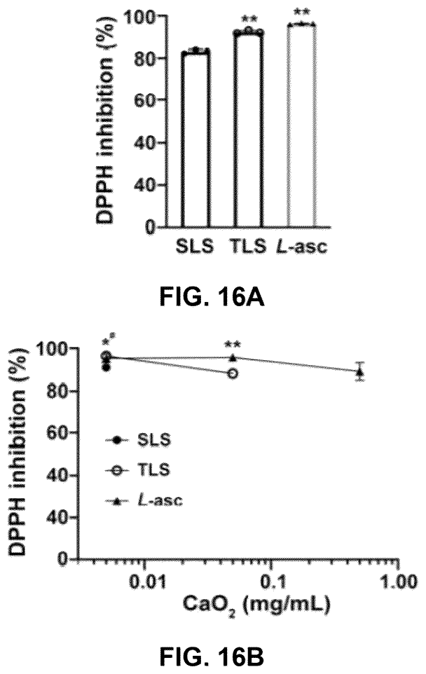

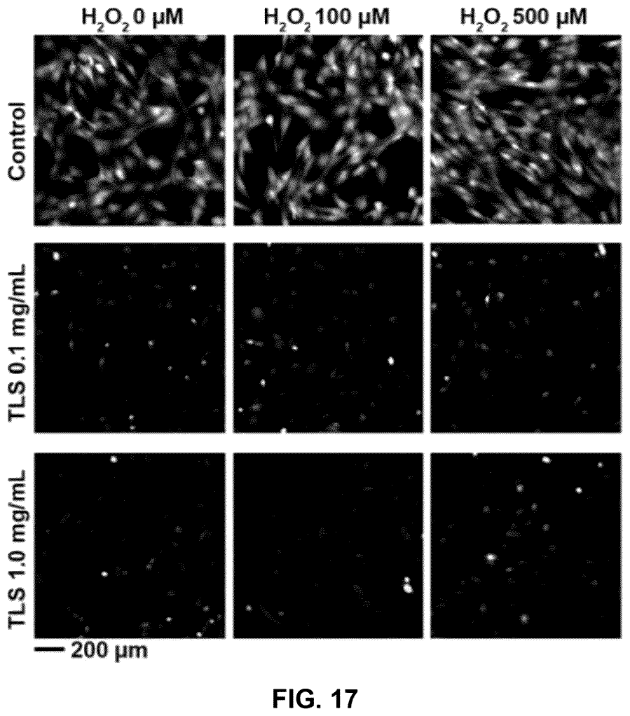

Incorporation of CaO₂ into NPs of SLS hydrophilic outer layer and PLGA hydrophobic core allows CaO₂ to be complexed with PLGA. When reacted with water at pH lower than 12 (FIG. 15A), CaO₂ can be decomposed into hydrogen peroxide, hydroxide ions and carbonate, and the generated hydrogen peroxide further decomposes into highly reactive superoxide and hydroxyl radical. Catalase can decompose the intermediate hydrogen peroxide into water and oxygen. While catalase is an enzyme found in the blood and liver of mammals, this enzyme has to be available in situ, not from peroxisome to prevent damages from ROS. This intermediate step eliminates any potential cytotoxic ROS. Without catalase, the cytotoxic byproduct H₂O₂ may lead to cell damage. Although the actual role of catalase in the oxygen-release process is unclear, decomposition is suggested to take place through the Modified Fenton chemistry-dissociation of H₂O₂ to OH radicals with Fe^{2+/3+}. These oxidants react with everything within diffusion limit layer while their half-lives are short. In wound healing, the availability of catalase is limited. Even though the level of expression of catalase mRNA is not changed during wound healing, the protein level of catalase is decreased. For example, catalase concentration is reported to be in the range of 50-100 U/mL in the oxygen generating gelatin section. In the mixture of CaO₂, catalase and either SLS or TLS, the dissociation of H₂O₂ is predominantly facilitated by catalase and partly by TLS and to less extent by SLS (FIG. 15B). The native antioxidant properties of SLS or TLS NPs (FIG. 16A) are maintained at levels of 80% or above of the native antioxidant (L-ascorbic acid) in the presence of up to 50 μg/mL of CaO₂ (FIG. 16B). TLS also showed the ROS scavenging capability in cultures after treating C2C12 myocytes with H₂O₂ (FIG. 17). It was found that the extent of fluorescence intensity from DCFDA is much diminished in the presence of TLS. Since complete removal of ROS is also detrimental in wound healing, the residual ROS detected by the DCFDA assay is indispensable for the survival of C2C12 myocytes. These results implicate that lignin composites effectively scavenge excessive ROS in the earlier phase of wound healing, leading to improved healing of the wounds in vivo.

Several cases of CaO₂ incorporation to gelatin-based biomaterials are reported to date. A recent study utilized microparticles (MPs) formed with the encapsulation of CaO₂ in PCL (polycaprolactone). These particles incorporated in composites with GelMA and release O₂ up to 5 weeks. Over 2 weeks, 2.5 UM of H₂O₂ is released without the formation of MPs while 1.0 UM of H₂O₂ with the formation of MPs. With 5 to 20 mg/ml of CaO₂, the cumulative release of O₂ is 25% without MP and up to 30% with MP. Some cases include catalase (100 U/mL) and CaO₂ (up to 30 mg/mL). GelMA (50 mg/rnL)-CaO₂ (30 mg/mL) with catalase (100 U/mL) releases O₂ up to 5 days. Another type of hydrogel utilizes Ca²⁺ from CaO₂ (up to 10 mg/mL) to crosslink gellan gum (anionic polysaccharide) with catalase. Thiolated gelatin (27 to 63 mg/mL) with CaO₂ (25 to 100 mg/mL) and catalase (2000-5000 U/mg) forms hyperbaric oxygen generating biomaterials. The question is how much CaO₂ is required to efficiently deliver O₂ to tissue microenvironments without causing cytotoxicity. CaO₂ concentrations higher than 10 mg/mL exhibit cytotoxicity to 3T3 FBs. In addition, due to low solubility in water, it is difficult to disperse Ca_{O}2 in aqueous buffers. Further, Ca²⁺ released from these composites influence both cell cytotoxicity and bacterial adhesion. Therefore, in these studies, lower CaO₂ was incorporated in the CPO lignin composites than other gelatin-based, oxygen generating biomaterials, which resulted in at least 7 days of sustained delivery of O₂.

To delineate the difference, the release kinetics of O₂ and H₂O₂ from CaO₂ in water with varying pH and temperature is probed and modeled. The release of H₂O₂ follows pseudo-zero order reaction. Once CaO₂ is dissolved in water, Ca2+, O₂²⁻ and H⁺ are formed. Of these, 2H⁺ and O₂²⁻ form H₂O₂. With increase of H⁺ (decrease of pH), the solubility of CaO₂ is increased. Temperature has minimal to no effect in the kinetics. In contrast, the release of O₂ follows a pseudo-first order reaction, meaning that the concentration of CaO₂ directly affects the dissociation of CaO₂ to O₂. With the decrease of pH, the release of O₂ is decreased. With the increase of temperature, the release of O₂ increases. From this study of kinetics, it is surmised that H₂O₂ is not necessarily a precursor of O₂.

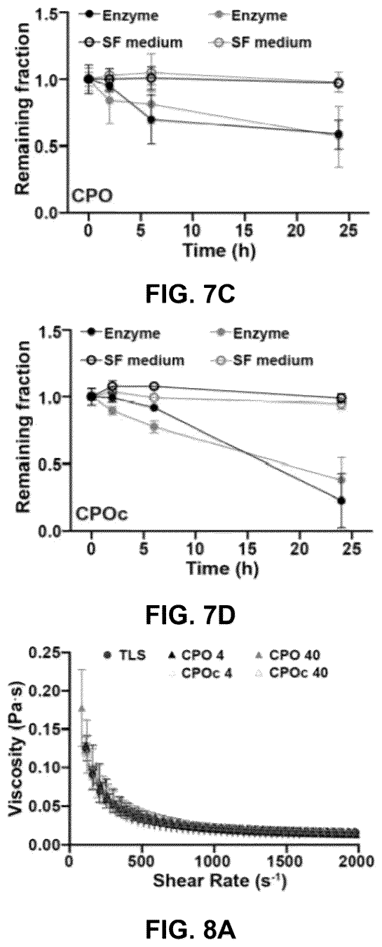

Swelling ratios and rate of degradation were not significantly different with respect to the presence or the concentrations of NPs of SLS-PLGA/CaO₂: Lignin composites in the wound microenvironment will be subject to swelling and enzymatic degradation and undergo remodeling. Thus, the extent of swelling of lignin composites was assessed by varying the concentration of incorporated NPs of SLS-PLGA with or without CaO₂. As shown in FIG. 7A, the swelling ratios between CPO and CPOc lignin composites were not significantly different at both 4 and 40 mg/mL. With 40 mg/mL of NPs of SLS-PLGA in lignin composites, the swelling ratios were slightly reduced by around 7% (CPO) and 8% (CPOc) without any statistical difference. Apparently, the 10 times higher mass fraction of NPs (regardless the presence of CaO₂) in lignin composites contributed to the swelling behavior with marginal difference. To determine the fraction of remaining lignin composites, TLS, CPO (4 and 40 mg/mL), and CPOc (4 and 40 mg/mL) lignin composites were submerged either in collagenase/CaCl₂)/SF medium or CaCl₂)/SF medium. After 24 h, less than 28% of TLS lignin composite (FIG. 7B), 38% of CPOc lignin composite with NPs of SLS-PLGA (w/o CaO₂) at 40 mg/mL and 23% of CPOc lignin composite with NPs of SLS-PLGA (w/o CaO₂) at 4 mg/mL remained (FIG. 7D), while CPO lignin composites retained around 60% at both 4 and 40 mg/ml (FIG. 7C). All control groups (SF medium in FIGS. 7B-7D) showed minimal (up to 7%) to no changes in the remaining fraction of lignin composites. The concentration of the collagenase type II used here was 0.5 U/rnL (equivalent to 430 μg/rnL), which is several orders of magnitude higher than the reported concentrations in patient tissues. For example, the concentration of matrix metalloproteinases (MMP)-1 and -9 in diabetic foot wounds are estimated between 20-100 ng/ml. However, since the concentrations of the MMPs in inflamed tissues vary from patient to patient, the higher collagenase concentration utilized in this study will still cover the ranges that may be encounter in vivo under different conditions. Furthermore, native or wounded skin has a plethora of MMPs, their inhibitors, and serum proteins, which leads to a tighter control of degradation and thus, it is expected slower degradation of lignin composites in vivo. Nevertheless, a certain extent of degradation of lignin composites (primarily, GelMA) is expected in vivo to transiently protect NPs of SLS-PLGA/CaO₂ from rapid, enzymatic, and mechanical degradation, while promoting antioxidant activity from lignosulfonate (both NPs of TLS and SLS in SLS-PLGA).

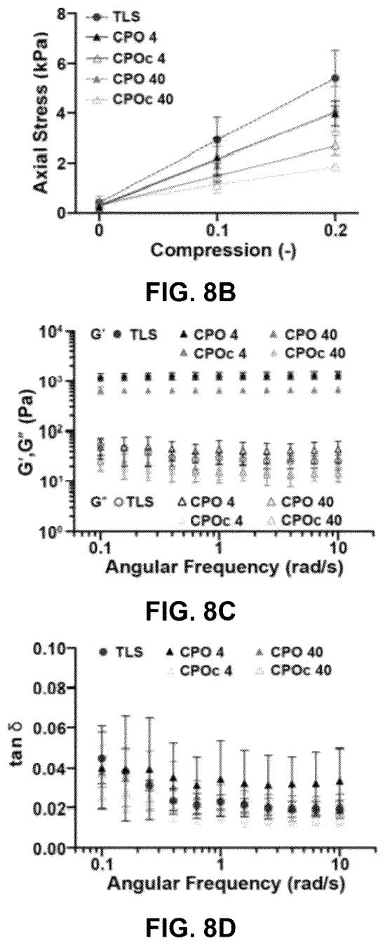

NPs of SLS-PLGA did not alter the viscosity of the lignin composite precursors while quantity and type of NP modulated the viscoelasticity of lignin composites: Since lignin composites are subject to needle injection to the wounded areas, the mechanical properties of lignin composites were assessed before and after thiol-ene crosslinking. Viscosity of all five different types of lignin composite precursors was tested and no significant difference was found (FIG. 8A). Apparently, crosslinked lignin composites exhibited similar viscoelasticity. However, the compressive modulus of elasticity was significantly different upon incorporating NPs of SLS-PLGA/CaO₂ and SLS-PLGA (w/o CaO₂). As shown in FIG. 8B and Table 5, TLS lignin composites exhibited the highest modulus of elasticity. Upon incorporating NPs of SLS-PLGA/CaO₂ at 4 or 40 mg/rnL, moduli of elasticity were decreased to around 18-19 kPa. While TLS is thiolated SLS to apply thiol-ene crosslinking, SLS in NPs of SLS-PLGA was not functionalized for crosslinking. Instead, NPs of SLS-PLGA/CaO₂ harness CaO₂ proximal to PLGA chains, while NPs of SLS-PLGA (w/o CaO₂) were formed without any CaO₂, which may form cavities during lyophilization. This possibly resulted in lowering the elasticity from the compression test during oscillating rheometry. Consequently, higher quantity (40 mg/mL) of NPs of SLS-PLGA (w/o CaO₂) showed much lowered slope down to 7.27 kPa in comparison to that of CPO or TLS lignin composites. In FIG. 8C, storage moduli of TLS and CPO lignin composites were similar to each other while those of CPOc lignin composite were significantly different from those of TLS or CPO lignin composites. However, loss tangent (G″/G′) of all lignin composites ranged from 0.01 to 0.07 (equivalent to 8 (phase lag) ranging from 0.57° to 4°, indicative of well-crosslinked viscoelastic composites (FIG. 8D). Collectively, the reduction of stiffness by non-cross linkable NPs of SLS-PLGA/CaO₂ is potentially significant at 40 mg/mL, thus the investigation of oxygen generating capability from lignin composites with NPs of SLS-PLGA (with or without CaO₂) at 4 mg/mL in mouse models of wound healing was continued.

TABLE 5
Elastic Modulus Estimated by the
Slope of Axial Stress vs. Compression

| Lignin | | Slope (Elastic | Intercept | |
|---|---|---|---|---|
| Composite | | modulus, kPa) | (kPa) | R² |
| TLS | | 24.9 | 0.430 | 0.8960 |
| CPO 4 | mg/mL | 18.7 | 0.286 | 0.9265 |
| CPO 40 | mg/mL | 18.4 | 0.329 | 0.8661 |
| CPOc 4 | mg/mL | 11.8 | 0.304 | 0.9557 |
| CPOc 40 | mg/mL | 7.27 | 0.409 | 0.9290 |

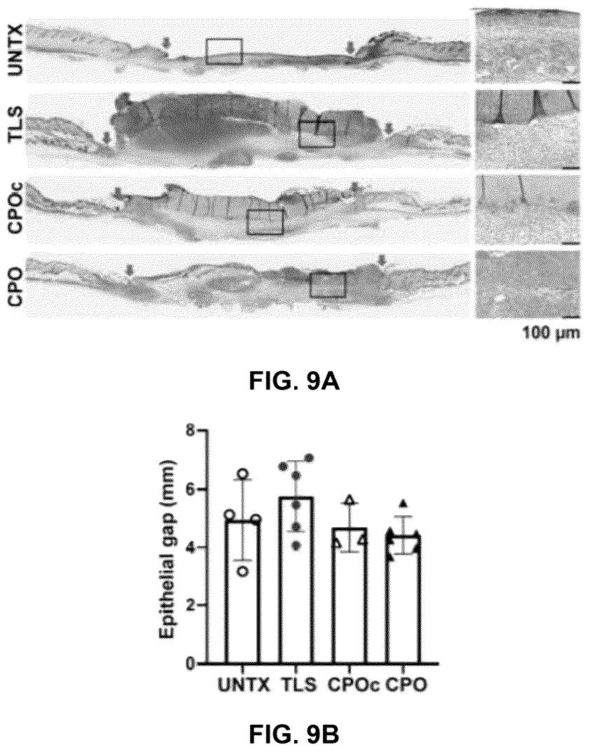

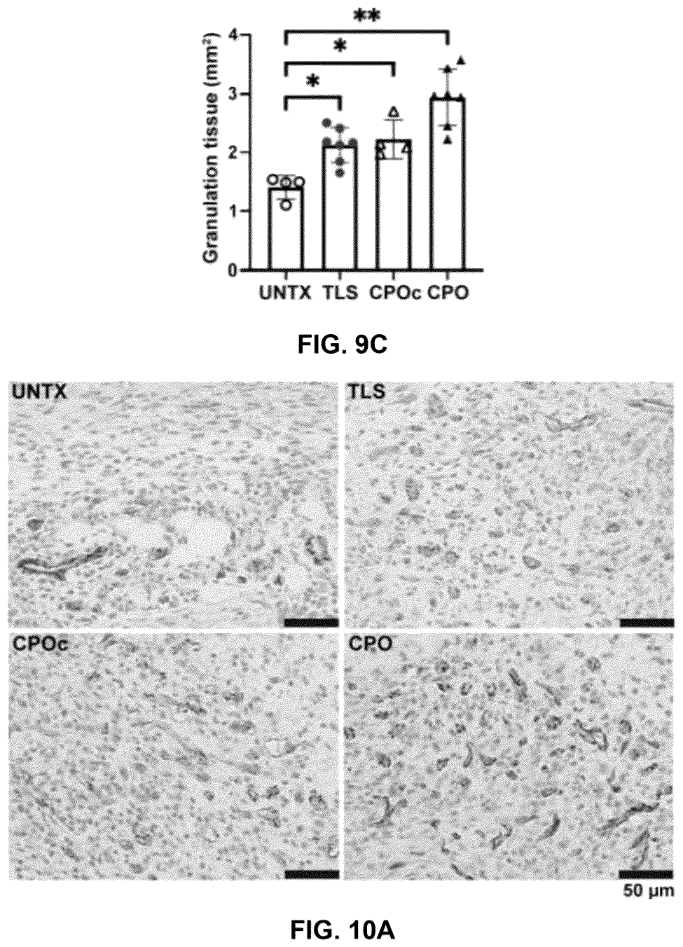

The granulation tissue area was significantly increased with CPO lignin composites: To determine the effect of lignin composites on the progression of wound healing, wounds were examined on alternate days for the first 7 days post wounding, which represents the early healing stages of inflammation and proliferation. Photographs of the wounds showed that mice tolerated the treatment with different lignin composites well, without any noticeable exudates (FIG. 13). Most of the wounds still bad the lignin composite hydro gels topically visible even until day 7, thus assessment of wound closure rate using planimetry to calculate the area of open wounds from the wound pictures was not performed. Wounds were harvested at day 7 post-wounding to examine re-epithelialization and granulation tissue formation using H&E staining and morphometric image analysis. Untreated wounds (UNTX in FIG. 9A) showed granulation tissue formation and certain extent of re-epithelialization with encroaching epithelial margins (indicated in arrows) with an open wound as expected of the stented murine WT wounds at day 7. Wounds treated with TLS or CPOc lignin composites similarly showed granulation tissue formation and certain extent of re-epithelialization, however, they still showed the separation of composite matrix from the wound bed tissue (insets in FIG. 9A). The CPO lignin composites, in contrast, showed enhanced integration with the granulating wound bed, with cell infiltration uniformly prevalent across the wound cross-section. There was no difference noted in the rate of the wound closure, as determined by epithelial gap (the distance between the encroaching epithelial margins indicated by the arrows), between UNTX and TLS (5.0+1.4 vs. 5.8±1.2 mm, p>0.05), CPOc (5.0±1.4 vs. 4.7±0.8 mm, p>0.05), or CPO (5.0±1.4 vs. 4.4±0.7 mm, p>0.05) lignin composite treated mice (FIG. 9B). However, significantly increased granulation tissue area was observed, between UNTX and TLS (1.4±0.2 vs. 2.1±0.3 mm, p=0.01) or CPOc (1.4±0.2 vs. 2.2±0.3 mm, p=0.01) lignin composite treated mice and more in CPO lignin composite treatment (1.4±0.2 vs. 3.0±0.5 mm², p<0.01) (FIG. 9C), which is indicative of healthy wound healing progression in all the lignin treated wounds.

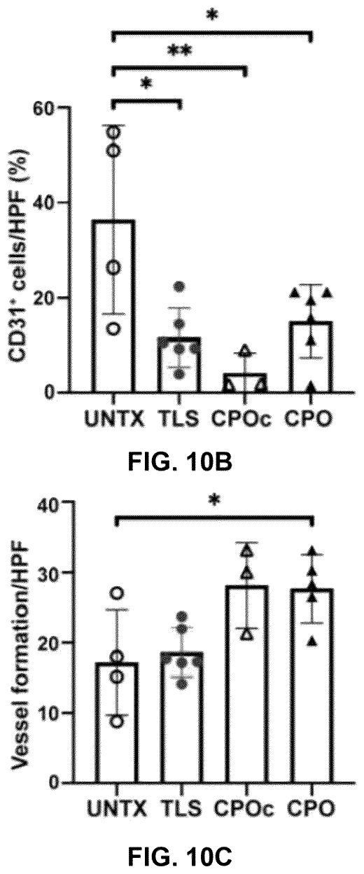

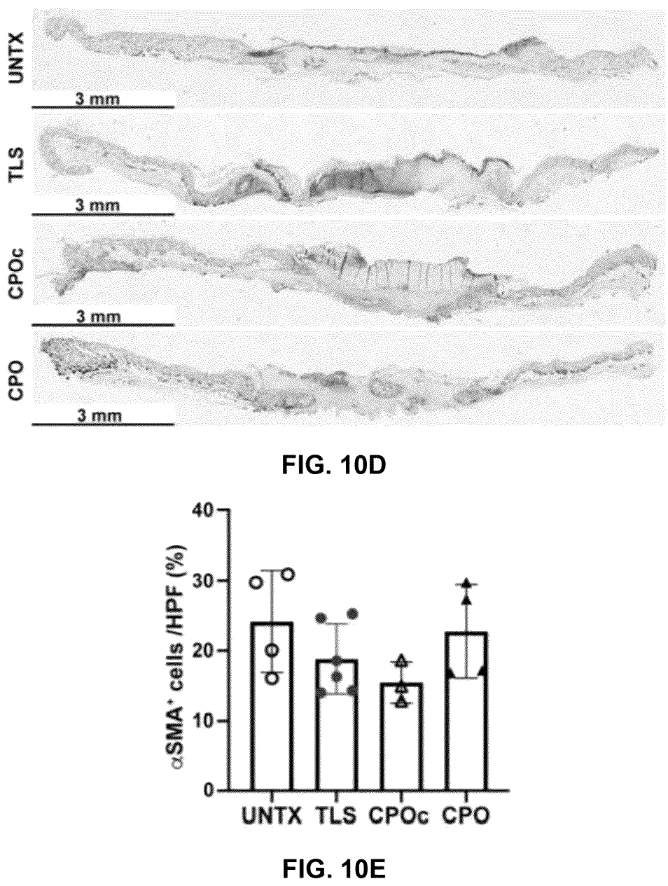

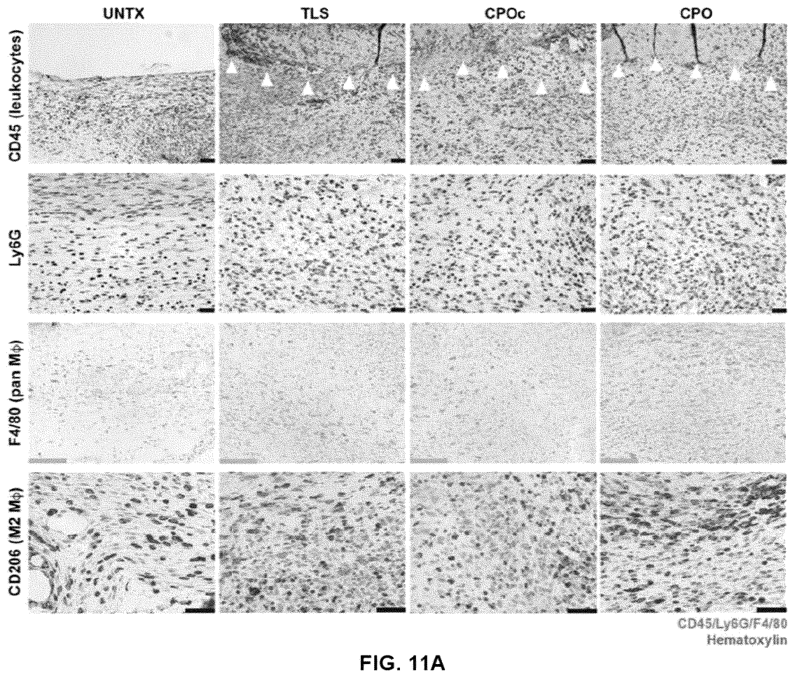

CPO lignin composites promoted wound neovascularization: Since neovascularization of the granulating wound bed is a key indicator of wound healing progression, wound tissue sections were stained with CD31, a marker of ECs. In addition to the increase in granulation tissue formation, neovascularization was also promoted by the CPO lignin composites. CD31 staining revealed that UNTX wounds had a higher number of individual CD31⁺ ECs when compared to lignin composite treatments, while the formation of capillary lumens was lower in UNTX wounds at day 7 in these wounds (FIG. 10A). Quantification of the CD31⁺ cells per HPF that were not associated with the lumens were first counted, which showed significantly higher counts per HPF in UNTX wounds than TLS-(36.4±19.8 vs. 11.6±6.3%, p<0.05), CPOc-(36.4±19.8 vs. 4.2±4.3%, p<0.01) or CPO-(36.4±19.8 vs.15.1±7.7%, p<0.05) lignin composite-treated wounds (FIG. 10B). Quantification of capillary lumen density per HPF showed significantly fewer lumens in UNTX. However, there were significantly more capillary lumens per HPF in the CPO lignin composite-(17.3±7.6 vs. 17.3±7.5 vessels/HPF, p<0.05) treated wounds as compared to UNTX or TLS wounds (FIG. 10C).

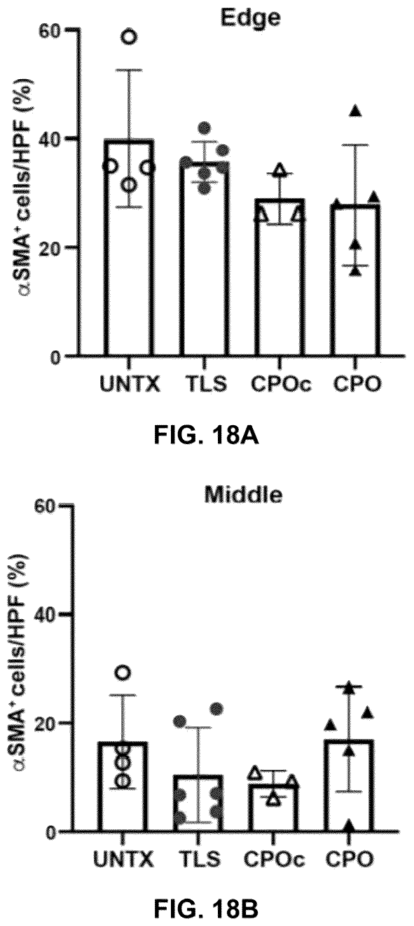

In addition, sections were stained for aSMA to detect myofibroblasts in the wounds. As shown in FIG. 10D, aSMA⁺ cells were limited to the edge of wounds in UNTX and TLS lignin composites. CPO or CPOc lignin composites showed more aSMA⁺ cells across the whole wound bed. While the extent of cells at either the edges or middle of the sections was not significantly different among the treatments (FIGS. 18A-18B), the overall trend indicated a slight increase in the aSMA⁺ cells in CPO lignin composites (FIG. 10E). Of note, the CPO lignin composite-treated wounds revealed a higher number of aSMA⁺ lumens both at the edge and the middle of wounds (FIG. 10D). Review of the literature indicate that aSMA is present in pericytes on capillaries and its expression in capillary vessels is associated with the development of vasculature. The expression of aSMA⁺ by myofibroblasts has also been shown to underlie tissue regeneration in the skin, and the number of aSMA⁺ cells decrease as the regeneration process is completed. Thus, these data show that neovascularization is promoted, and the wound healing is promoted but still in progress by day 7 with the treatment with CPO lignin composite.

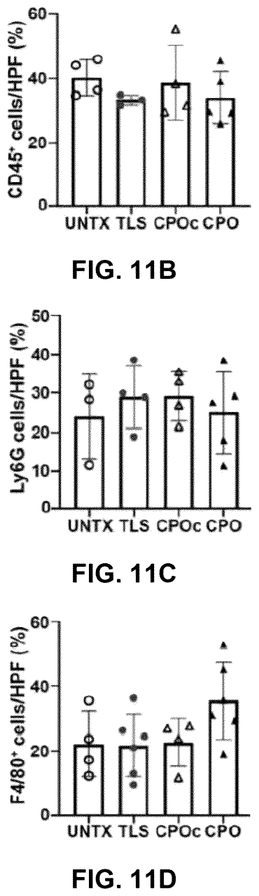

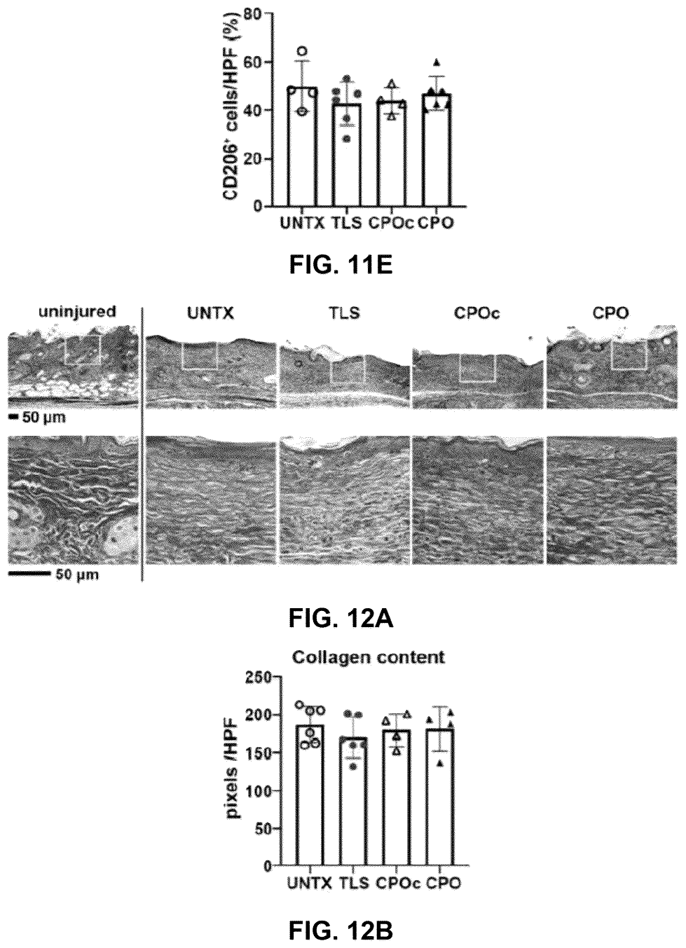

Lignin composites did not cause significant inflammatory responses in the dermal wounds: Inflammatory responses in the wounds in response to lignin composite treatments was assessed by immunohistochemical staining of wounds sections with a panel of inflammatory markers (FIGS. 11A-11E). CD45 is expressed by common leukocytes except platelet and red blood cells. Ly6G is expressed by monocytes, granulocytes and neutrophils. F4/80 is used to identify tissue macrophages. CD206 is normally expressed on the alternatively activated, anti-inflammatory (M2) macrophages. At 7 days post wounding, no significant changes in the expression in any the four markers were noted.

As shown in FIG. 11A, the interface between wounds and lignin composites does not show (indicated by white wedges) significant infiltration of CD45⁺ cells. There was no significant difference in the percentages of CD45⁺ (pan-leukocyte) cells/HPF across all tested lignin composites when compared to UNTX wounds (UNTX 40.3±5.7 vs. TLS 33.3±1.5 vs. CPOc 38.7±11.7 vs. CPO 34.0±8.2%, p>0.05) (FIG. 11B). A similar trend was observed in monocyte, granulocyte, and neutrophil infiltration as determined through Ly6G staining of the wounds (FIG. 11C). There were no significant differences in the percentage of Ly6G⁺ cells/HPF among treatment groups (UNTX 24.0±11.0 vs. TLS 29.1±8.1 vs. CPOc 29.3±6.3 vs. CPO 25.1±10.5, p>0.05). No significant differences were observed in macrophages either. F4/80 and CD206 staining (FIGS. 11C-11D) showed that UNTX wounds had no statistically significant differences in the levels of F4/80⁺ cells per HPF (UNTX 22.3±10.0 vs. TLS 21.9±9.6 vs. CPOc 22.8±7.3 vs. CPO 35.6±12.0%, p>0.05) or CD206⁺ cells per HPF (UNTX 49.9±10.5 vs. TLS 42.8±8.9 vs. CPOc 44.0±5.5 vs. CPO 47.0±7.2%, p>0.05).

Recently, the stiffness of GelMA has been shown to direct the macrophage phenotypes. Soft GelMA matrix (stiffness less than 2 kPa) is more favorable for priming macrophages toward M2 phenotypes with a decreased capacity for spreading in comparison to stiff (over stiffness 10 kPa) GelMA matrix. The lignin composites used in these wound healing studies are in the range of 1-2 kPa of storage modulus (FIG. 8C), thus likely priming macrophage toward anti-inflammatory (M2) phenotypes. While the markers to identify the inflammatory cells' types, particularly those associated with macrophages work very well for identifying macrophages using in vitro polarization with defined stimuli, there is evidence from literature that macrophages do not respond to biomaterials in the same way as they do to the biochemical stimuli with distinct polarizations. For example, Graney et al. investigated the behavior of macrophages cultured on ceramic-based scaffolds and found hybrid activation states that were not distinctly MI, or M2a, or M2c and further noted some markers were upregulated, while others were downregulated in their various scaffold studies, which is not possible to distinguish by cell counting from histologic sections. These studies indicate that increased numbers of phenotype markers are needed to capture the increase in complexity in macrophage phenotypes in biomaterial studies. In this regard, gene expression is being pioneered to characterize macrophage phenotypes more thoroughly. In particular, alternatively-activated M2 macrophages are important mediators of successful wound healing, and a thorough evaluation of their subtypes in lignin composite wounds would elucidate the mechanisms of its action on promoting wound healing.

In tissue engineering, lignin composites can be applied to enhance mechanical properties with good protein adsorption capacity and wound compatibility, to confer anti-inflammatory properties by reducing gene expression of iNOS and IL-1B of macrophages, to apply for wound-dressings with biocompatibility and nontoxicity and to achieve enhanced mechanical properties and viability of cells for direct ink writing 3D bioprinting. Nevertheless, toxicological studies of lignin have only been carried out through simple cytotoxicity testing, which cannot accurately simulate the human body environment. Thus, the biological effects of lignin with animal models should be further tested to assess the long-term stability and potential negative effects of lignin and lignin-derived biomaterials. In addition to toxicological studies, another difficulty and key point for the development of lignin-based biomaterials is the heterogeneity of lignin. Although lignin displays good potential for biomedical applications, its broad distribution of molecular weight and complex structures represent a hurdle for cross-validation deep studies by different groups, standardization, and scale-up. Thus, lignin chemistry and de/polymerization techniques still need to be continuously developed.

Wounds treated with the CPO lignin composite had minimal scarring and exhibited mature collagen architecture at 28 days post wounding: As a part of the wound-healing process, murine postnatal skin generally develops scar by 4 weeks post-wounding. Therefore, wounds at 4 weeks after treatments were stained with trichrome to examine dermal architecture and collagen expression. Uninjured ‘normal’ skin has distinct layers of the epidermis, the dermis is comprised of the papillary, reticular, and hypodermal subdivisions and also include the dermal appendages such as hair follicles and sweat gland etc.; then there is a distinct adipose layer and the panniculus carnosus muscle layer in the murine skin. Importantly, the dermal collagen demonstrates a basket-weave pattern in normal skin that renders the skin its stretch and strength. In contrast, scar tissue that forms after injury lacks dermal appendages, and most often, the adipose and panniculus layers also do not reform.

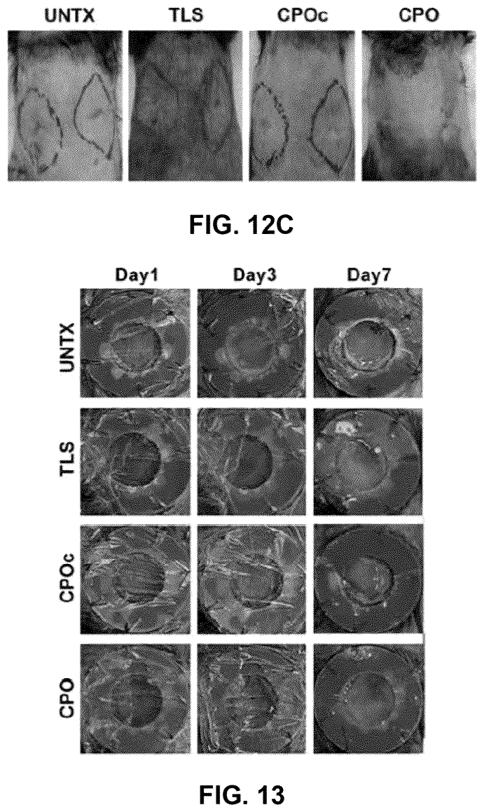

The architecture is also distinct in the scars, with dense ECM and parallel fibers of collagen approximately parallel to the epithelial basement membrane (FIG. 12A). Treatment of the wounds with the CPO lignin composite resulted in a smaller scar compared to untreated or TLS and CPOc treatments. There was a notable reconstitution of dermal appendages in the CPO wounds and traces of the panniculus carnosus muscle layer. The architecture of the collagen also showed a bundled mesh network like basket-weave, as opposed to relatively straight fibers found in TLS or CPOc lignin composites (FIG. 9A). However, measurements of the collagen content (positive pixels per HPF) did not show significant differences between treatment groups (FIG. 9B). No differences were observed in the overall collagen density between UNTX and TLS-(187.0±23.8 vs. 170.0±26.4 pixels/HPF, p>0.05), CPOc-(187.0±23.8 vs. 179.6±21.5 pixels/HPF, p>0.05), or CPO-(187.0±23.8 vs. 180.9±29.4 pixels/HPF, p>0.05) lignin composite-treated wounds. This data suggests that the strength of the wounds is not compromised with these treatment groups. Further, wounds treated with the CPO lignin composite left minimal scar as evidenced in photographs taken at 28 days after surgery (FIG. 9C). While pronounced differences in the collagen content of wounds treated with lignin composites were not observed, this was not surprising. Recently, a more robust analyses of murine scars are being pioneered by different groups on identifying how closely the collagen fiber density, packaging, the presence of dermal appendages, and epidermal topology of the scar resemble that of the normal skin, as these measures are important for the stretch and strength of the repaired skin tissue. In a study by Mascharak et al., a machine learning algorithm was used to quantify tissue ultrastructure. The latter study utilized Picrosirius Red stained sections, and the images of the wound cross-section were color-deconvoluted to isolate ECM fiber components to digitally map thousands of fibers and branchpoints. Individual (e.g., length, width) and group (e.g., packing, alignment) fiber properties were calculated, and skin and scars were compared across multiple metrics as opposed to collagen content. However, this is not yet widely available, and qualitative dermatopathological analysis of the histologic sections remain main stay. Future studies can similarly utilize advanced staining and imaging techniques and machine learning to differentiate the types of collagens (i.e. Type I vs. Type III), proteoglycans and other ECM components in the lignin composite wounds. Accordingly, these assessments can further aid in the optimization of the biomaterial properties to accelerate regeneration of the wounds with lignin composites.

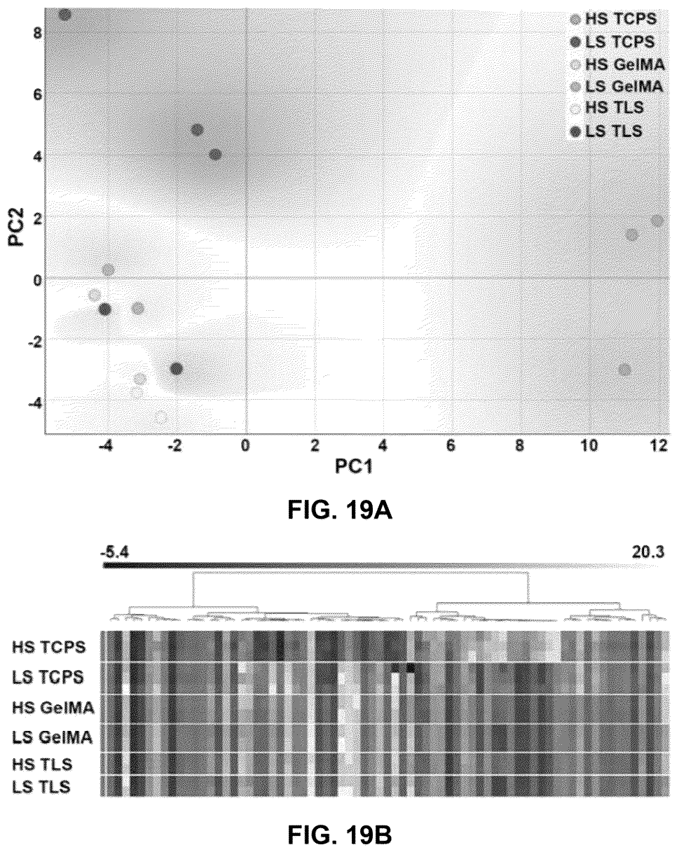

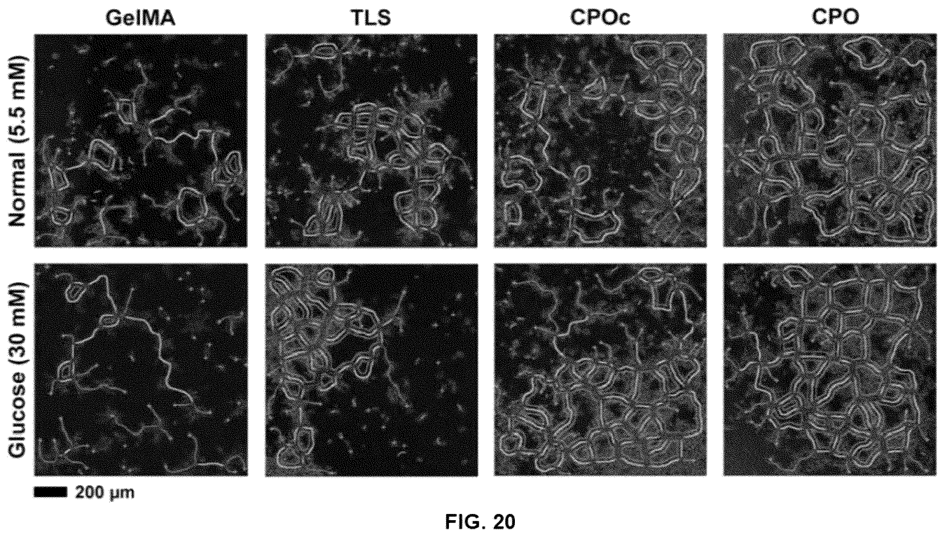

Our in vivo findings also corroborate previous work that showed that the antioxidation capacity of lignin attenuated the expression of fibrotic markers including COL1A1, ACTA2, TGFB1, and HIF1A in human dermal fibroblasts (hdFBs). Indeed, different patient derived fibroblasts from low to high scarring phenotypes were tested and showed that lignin composites attenuated the fibrotic markers in high scar-derived fibroblasts comparable to the phenotype of the low scar-derived group. To further confirm the attenuation of fibrotic phenotypes of LS (low scar-derived fibroblast) and HS (high scar-derived fibroblast) by lignin composites, 84 genes associated with angiogenesis were analyzed, ECM production and oxidative stress using a fibrosis PCR array. It was hypothesized that exposure of LS and HS hdFBs to TLS lignin composite will attenuate the fibrosis and oxidative stress response genes. As shown in FIGS. 19A-19B, PCA plots showed distinct differences among the HS and LS hdFBs when they were cultured on tissue culture plastic, without any overlap. However, the expression of fibrotic genes in both HS and LS hdFBs on lignin composites was significantly altered, and HS phenotype appeared to closely associate to that of the LS. Some of the key genes that were altered among the lignin treated and untreated groups included ECM producing genes such as COL1A1 and TGFB1 and ECM remodeling genes including TIMPs and MMPs. These results, together with the previous works suggest that addition of lignosulfonate to the wound healing microenvironment may attenuate fibrotic responses during tissue repair by modulating fibroblast phenotypes. This is area of prime interest for wound healing, with recent evidence of the involvement of distinct fibroblast lineages in the scar formation processes in wounds.

Although WT mice are programmed to heal physiologically with minimal alterations in perfusion and in the presence of ROS, improvement was seen in these measures of wound healing (FIGS. 9A-9C) and vessel formation (FIGS. 10A-10E) without significant inflammation (FIGS. 11A-11E). While the quantity of released oxygen from the lignin composites investigated here was around 700 ppm per day in vitro, this lignin composite formulation provided an effective dose of oxygen without causing excessive scarring in vivo, which is plausible by oxygen-mediated increased in VEGF stimulation Thus, it is surmised that the enhanced wound healing by the CPO lignin composite is the product of enhanced vascularization and granulation tissue formation. The translatable benefit of the lignin composite may be more readily apparent in disease states such as diabetic wound healing and/or with infection. The advanced glycation end products (AGEs) present in diabetic tissue generate ROS leading to apoptosis of the FBs via the NLRP3 (NOD-, LRR- and pyrin domain-containing protein 3) signaling pathway, thus impairing wound repair. Thus, some of the systemic oral anti-hyperglycemics currently used to treat diabetes mellitus also have antioxidant and beneficial wound healing effects. Metformin, for example, is a commonly used medication used for glucose control in diabetes mellitus patients which has antioxidant effects and demonstrated benefits on angiogenesis and wound closure in diabetic mice. However, as previously mentioned, pure systemic antioxidants have a poor enteric absorption profile. Locally applied antioxidant hydrogels have been demonstrated to accelerate diabetic wound healing while promoting M2 macrophage differentiation and reducing IL-1β production. Further, topical oxygen delivery to ischemic wounds is shown to accelerate healing and promote granulation tissue formation. Thus, the strategy of combining these dual functioning, wound-beneficial attributes into one local therapy for pathologic wounds has the potential to greatly benefit the wound healing in at-risk patients.

While collagen content was not significantly modulated, the wounds treated with CPO lignin composite exhibited mature collagen architecture over 30 days. Tissue sections were prepared to assess collagen architecture and content over 30 days of healing process from trichrome staining. In rodents, normal tissue has a reticular collagen pattern, whereas the collagen in scar tissue forms large parallel bundles at approximately right angles to the basement membrane. The CPO lignin composite exhibited more pronounced, bundled mesh network as opposed to relatively straight fibers found in TLS or CPOc lignin composites (FIG. 12A). Despite qualitative difference of collagen architecture, the quantity of collagen content over 30 days becomes similar to each other (FIG. 12B). No differences were observed in the overall collagen density between UNTX and TLS-(187.0±23.8 vs. 170.0±26.4 pixels/HPF, p>0.05), CPOc-(187.0±23.8 vs. 179.6±21.5 pixels/HPF, p>0.05), or CPO-(187.0±23.8 vs. 180.9±29.4 pixels/HPF, p>0.05) lignin composite-treated wounds. However, reduction of collagen content in a scar has less impact on scarring outcome compared to the structure of matrix deposited in the dermis. Further, wounds treated with the CPO lignin composite left minimal scar as evidenced in photographs taken at 30 days after surgery (FIG. 12C).

Application of polyphenolic lignin polymer to wound healing and tissue engineering applications is increasing. Application of lignin is ever increasing in a number of industrial and biomedical applications, including UV absorption, Cu²⁺ chelating in water, skin protectants, wound dressing with chitosan, self-healing smart biomaterials, durable and repeatable adhesive, antibacterial agent with metallic NPs, anti-proliferative effect to cancer cells without cytotoxicity. In tissue engineering, lignin composites can be applied to enhance mechanical properties with good protein adsorption capacity and wound compatibility, to provide 3D microenvironments of mesenchymal stromal cell culture, to confer anti-inflammatory properties by reducing gene expression of iNOS and IL-1β of inflamed mouse macrophages, to apply for wound-dressing with biocompatibility and nontoxicity, to improve mechanical properties and wound healing capabilities on incision-induced Sprague-Dawley rats, to provide nanofibers for the cultures of hdFBs, to promote localized and prolonged antioxidant capabilities for wound healing applications, and to achieve enhanced mechanical properties and viability of hdFBs for direct ink writing 3D bioprinting. Nevertheless, toxicological studies of lignin have only carried out through simple cytotoxicity testing, which cannot accurately simulate the human body environment. Thus, the biological effects of lignin with animal models should be tested to assess the long-term stability and potential negative effects of lignin and lignin-derived biomaterials. In addition to toxicological studies, another difficulty and key point for the development of lignin-based biomaterials is the heterogeneity of lignin. Although lignin displays good potential for biomedical applications, its broad distribution of molecular weight and extremely complex structures represent big hurdles for deep studies, standardization, and scale-up. Thus, lignin chemistry and de/polymerization techniques still need to be continuously developed.

CaO₂ is applicable for oxygen generating compounds for tissue regeneration. The injectable lignin composite can be a unique biomaterial platform to confer antioxidation by TLS and oxygen generation from NPs of SLS-PLGA/CaO₂ without incorporating additional enzymes. Incorporation of CaO₂ into NPs of SLS hydrophilic outer layer and PLGA hydrophobic core allows CaO₂ to be complexed with PLGA. CaO₂ is a white to yellowish powder which has been extensively applied in water treatment, seed disinfection and food processing. When reacted with water at pH lower than 12 (FIG. 15A), CaO₂ can be decomposed into hydrogen peroxide, hydroxide ions and carbonate, and the generated hydrogen peroxide further decomposes into highly reactive superoxide and hydroxyl radical. Catalase can decompose the intermediate hydrogen peroxide into water and oxygen. While catalase is an enzyme found in the blood and liver of mammals, this enzyme has to be available in situ, not from peroxisome to prevent damages from ROS. This intermediate step eliminates any potential cytotoxic ROS. Without catalase, the cytotoxic byproduct H₂O₂ may lead to cell damage. Although the actual role of catalase in the oxygen-release process is unclear, decomposition is suggested to take place through the Modified Fenton chemistry-dissociation of H₂O₂ to OH radicals with Fe^{2+/3±}. These oxidants react with everything within diffusion limit layer while their half-lives are short. In wound healing, the availability of catalase is limited. Even though the level of expression of catalase mRNA is not changed during wound healing, the protein level of catalase is decreased. For example, catalase concentration is reported to be in the range of 50-100 U/mL in the oxygen generating gelatin section or 1 mg/mL in the matrix of polycaprolactone (PCL). In the mixture of CaO₂, catalase, and either SLS or TLS, the dissociation of H₂O₂ is predominantly facilitated by catalase and partly by TLS and to less extent by SLS (FIG. 15B). The native antioxidant properties of SLS or TLS NPs (FIG. 16A) is maintained at levels of 80% or above of the native antioxidant (L-ascorbic acid) in the presence of up to 50 g/mL of CaO₂ (FIG. 16B).

Several cases of CaO₂ incorporation to biomaterials are reported to date. Injectable cryogels are formed with methacrylated hyaluronic acid and CaO₂. Cumulative release of H₂O₂ is up to 468 μmol over 3 h. Microparticles are formed with the encapsulation of CaO₂ in PCL, then formed composites with GelMA. Over 2 weeks, 2.5 UM of H₂O₂ is released without the formation of MPs while 1.0 UM with the formation of MPs. With 5 to 20 mg/ml of CaO₂, the cumulative release of O₂ is 25% without MP and up to 30% with MP. Some cases include catalase (100 U/mL) and CaO₂ (up to 30 mg/ml). GelMA (50 mg/mL)-CaO₂ (30 mg/mL) with catalase (100 U/mL) releases O₂ up to 5 days. Another type of hydrogel utilizes Ca2⁺ from CaO₂ (up to 10 mg/mL) to crosslink gellan gum (anionic polysaccharide) with catalase. In the matrix of silk/keratin/gelatin, CaO₂ up to 200 mg/mL release O₂ over 2 weeks with additional antibacterial properties for urethral tissue engineering applications. Thiolated gelatin (27 to 63 mg/mL) with CaO₂ (25 to 100 mg/mL) and catalase (2000-5000 U/mg) forms hyperbaric oxygen generating biomaterials. The question is how much CaO₂ is required to efficiently deliver O₂ to tissue microenvironments without causing cytotoxicity. CaO₂ concentrations higher than 10 mg/mL exhibit cytotoxicity to 3T3 FBs. In addition, due to low solubility in water, it is difficult to disperse CaO₂ in aqueous buffers. Further, Ca²⁺ released from these composites influence cell cytotoxicity and bacterial adhesion. To assess the kinetics of O₂, in situ oxygen measurement with patch sensors are used. In the case of measuring concentrations of H₂O₂, Cu(II)-neocuproine spectrophotometric method or free radical analyzer are used. To delineate the difference, the release kinetics of O₂ and H₂O₂ from CaO₂ in water with varying pH and temperature is probed and modeled. The release of H₂O₂ follows pseudo-zero order (PZO) reaction. Once CaO₂ is dissolved in water, Ca²⁺, O₂²⁻, and H⁺ are formed. Of these, 2H⁺ and O₂²⁻ form H₂O₂. With increase of H⁺ (decrease of pH), the solubility of CaO₂ is increased. Temperature has minimal to no effect in the kinetics. In contrast, the release of O₂ follows pseudo-first order (PFO) reaction, meaning that the concentration of CaO₂ directly affects the dissociation of CaO₂ to O₂. With the decrease of pH, the release of O₂ is decreased. With the increase of temperature, the release of O₂ increases. From this study of kinetics, H₂O₂ is not necessarily a precursor of O₂.

Locoregional delivery of antioxidation and oxygen generation could be beneficial to promote wound healing: Previous work showed that the antioxidation capacity of TLS attenuated the expression of COL1A1, ACTA2, TGFB1, and HIF1A and resulted in the similar extent of expression of the fibrotic markers comparable to proliferative LS hdFB phenotypes. To further confirm that the attenuation of fibrotic phenotypes of hdFBs by TLS lignin composite, 84 genes associated with angiogenesis were analyzed, ECM production and oxidative stress using a PCR array. It was hypothesized that exposure of LS and HS hdFBs to TLS lignin composite attenuates these angiogenesis, ECM production and oxidative stress responses of HS to the levels comparable to those of LS. As shown in FIGS. 19A-19B, the expression of fibrotic genes in HS hdFBs on lignin composites was significantly altered to mimic those in LS. These results and the previous works suggest that addition of lignosulfonate to the microenvironment of wound healing may attenuate fibrotic responses during tissue repair. Further, sustained supply of oxygen in vivo should be included for enhanced angiogenesis (FIGS. 10A-10E) and ECM production (FIGS. 12A-12B) using a biomaterial platform based on lignosulfonate. Although WT mice are programmed to heal physiologically with minimal alterations in perfusion and in the presence of ROS, improvement was observed in these measures of wound healing (FIGS. 9A-9C) and vessel formation (FIGS. 10A-10E) without significant inflammation (FIGS. 11A-11E). While the quantity of released oxygen (around 700 ppm per day) is suboptimal yet, the formulation of lignin composites investigated here is rather an effective suboptimal dose of oxygen without causing excessive scarring by VEGF stimulation. Thus, the enhanced wound healing by CPO lignin composite is the product of enhanced vascularization and granulation tissue formation.

The translatable benefit of the lignin composite may be more readily apparent in disease states such as diabetic wound healing and/or with infection. The advanced glycation end products (AGEs) present in diabetic tissue generate ROS leading to FB apoptosis via the NLRP3 (NOD-, LRR- and pyrin domain-containing protein 3) signaling pathway, thus impairing wound repair. Some systemic oral anti-hyperglycemics currently used to treat diabetes mellitus also have antioxidant and beneficial wound healing effects. Metformin, for example, is a commonly used medication used for glucose control in diabetes mellitus patients which has antioxidant effects and demonstrated benefits on angiogenesis and wound closure in diabetic mice. However, as previously mentioned, pure systemic antioxidants have a poor enteric absorption profile. Locally applied antioxidant hydrogels have been demonstrated to accelerate diabetic wound healing while promoting M2 macrophage differentiation and reducing IL-1β production. Further, topical oxygen delivery to ischemic wounds is shown to accelerate healing and promote granulation tissue formation. The strategy of combining these dual acting, wound-beneficial attributes into one local therapy for pathologic wounds has the potential to greatly benefit the wound healing in at-risk patients.

### Conclusions

Wound healing applications of lignin have been somewhat limited to doping lignin NPs into a matrix of biomaterials. Here, lignosulfonate was functionalized to form two different types of NPs, to scavenge ROS and to locoregionally deliver O₂ without adverse effect on tissue oxygenation. Injection of the CPO lignin composites to wounds resulted in multiple, positive wound healing responses in that significant increase in the area of granulation tissue formation and neovascularization were observed as evidenced by significantly increased blood vessel formation and the infiltration of aSMA⁺ cells to the wound by 7 days after surgery. Lignin composites did not cause significant inflammatory response, and the mechanical properties of the CPO lignin composites were amenable to direct M2-like macrophage phenotype induction in the wound microenvironments. By 28 days, the collagen architecture in the wounds treated with the CPO lignin composite exhibited more pronounced, bundled basketweave like network and left minimal scar. Thus, lignin based soft matrix with antioxidation (conferred by TLS) and synergistic oxygen release (conferred by SLS-PLGA/CaO₂ NPs) could be applied to wound healing applications with enhanced tissue granulation, vascularization, and maturation of collagen architecture without significant inflammatory responses.

### Supplementary Methods

Synthesis of lignin NPs: The coupling of lignosulfonate to PLGA was performed by acylation reaction for SLS in a mass ratio of 2 to 1. The final precipitate was collected and dried for 2 days under vacuum. Then, SLS-graft-PLGA (SLS-PLGA) was dissolved in ethyl acetate (EtOAc, organic phase) and calcium peroxide (CaO₂, Cat #466271, Sigma-Aldrich, St. Louis, MO, USA) and dissolved under stirring at room temperature. The organic phase was added to the aqueous phase (low resistivity water) under stirring. The suspension was homogenized with a microfluidizer (Microfluidics Corp., Westwood, MA, USA) at 30 kpsi at 4° C. Next, the organic solvent was evaporated with a rotary evaporator R-300 (Buchi Corp, New Castle, DE, USA). Finally, trehalose was added (1:1 mass ratio) as a cryoprotectant for the lyophilization of SLS-PLGA NPs and stored at −20° C. for further characterization and experiments.

Alkene incorporation reaction in gelatin: Alkene was incorporated into gelatin by the coupling reaction of gelatin with methacrylate acid (MA). Gelatin (1.00 g) was dissolved in 10.0 mL of PBS (pH 7.4) at 50° C. Separately, a solution of MA (0.10 g), N-ethyl-N′-(3-(dimethylamino)propyl)carbodiimide (EDC; 0.178 g), N-hydroxysuccinimide (NHS, 0.107 g) and dimethyl sulfoxide (DMSO, 2.00 mL) was prepared by stirring at 40° C. for 30 min. The flask containing the gelatin solution was placed in an oil bath at 50° C., and the other solution of MA, EDC, NHS, and DMSO was added dropwise. The mixture was further stirred at 50° C. for 1.5 h. The solution was cooled down to room temperature, followed by dialysis in water at 40° C. for a week. The resulting solution was lyophilized to yield alkene-incorporated gelatin powder sample. The coupling efficiency of MA to gelatin was assessed using 1H NMR comparing the phenyl peak (a) and the lysine peak (b) (FIG. 4). Upon coupling reaction, the development of two distinct peaks between 5 to 6 ppm were observed in 1H NMR spectrum, referring to the two protons in alkene group of MA attached to the lysine of gelatin. Additionally, the gelatin phenylalanine (Phe) group remained untouched during the reaction and allowed determination of the mmol/g ratio of the Gel MA. From literature, native porcine gelatin contains 15.5 mol/10⁵ g of Phe. Combining this information with the ratio of Phe:MA, the mmol/g concentration of MA was calculated using the following equation: MA concentration (mmol/g)=(15.5 mol/10⁵ g)×(1000 mmol/mol)×(Iₚₕₑ/Iₐₗₖₑₙₑ) (intensity ratio of phenyl to alkene). The batch presented in FIG. 4 showed the Iₚₕₑ/Iₐₗₖₑₙₑ of 1: 1.905 ([intensity from five protons of Phe/5]/[intensity of two protons of alkene/2]) resulting in a MA concentration of 0.295 mmol/g. This value also can be utilized to estimate the degree of substitution with a ratio of 1:2.20 ([intensity from five protons of Phe/5]/[intensity of two protons on carbon adjacent to NH₂ (c) in Lys/2)) of Phe:Lys of the pristine gelatin (i.e. [reacted MA upon the substitution]/[reactive amine site (Lys) in pristine gelatin]×100=1.905/2.20×100=87%), suggesting that the modifications made in this synthesis route produced GelMA with around 87% substitution.

Cultures of high and low scar normal human dermal fibroblasts: Skin tissue was collected from abdominoplasty patients who provided written informed consent as part of a protocol approved by the Institutional Review Board of Texas at Baylor College of Medicine in accordance with the Declaration of Helsinki. A biobank of scar and matched normal uninjured skin tissue was obtained from abdominoplasty patients controlled for sex, age, ethnicity, surgery type, indication, wound site, and comorbidities. These skin tissues were grouped into ‘low scarring’ and ‘high scarring’ phenotypes based on the evaluation of their existing c-section scars using Vancouver Scar Scale (VSS). Skin obtained from patients with 1-3 score on VSS were categorized as ‘low scarring’ phenotype (N) and patients with 6-9 score were grouped into ‘high scarring’ phenotype (S). For each group, n=3 skin tissues were pooled. Fibroblasts (FBs) were isolated from scar and matched normal skin cohorts and were called ‘low scarring’ (LS) and ‘high scarring’ (HS) normal fibroblasts based on their VSS phenotype and patient demographics. Only LS-normal and HS-normal hdFBs were used for cell studies, and they are denoted HS and LS for the rest of the studies. Both LS and HS hdFBs were maintained in DMEM (Cat #10567-014-500 mL, Thermo Fisher Scientific, Waltham, MA, USA) supplemented with 10% fetal bovine serum (FBS, Cat #35-015-CV, Corning, Corning, NY, USA), Penicillin/Streptomycin (P/S, Cat #15140-122, Thermo Fisher Scientific, Waltham, MA, USA), and antibiotic/antimycotic (anti/anti, Cat #15240-062, Thermo Fisher Scientific, Waltham, MA, USA). Only passages 8-10 were used for studies and cells were harvested at 80-90% confluency by applying TrypLE Express (Cat #12604-021, Thermo Fisher Scientific, Waltham, MA, USA) and pelleted at 350 g for 5 min. Supernatant was removed and hdFBs were gently resuspended in cell culture media and counted. Media was changed every other day.

PCR array: To determine the effect of lignin composite on their expression of fibrotic phenotype of hdFBs, LS and HS hdFBs were seeded onto composites at 50,000 cells/cm² and maintained for 24 h in a CO2 incubator (5% CO2/37° C.). For the fibrosis array, the concentrations of GelMA and LAP were fixed at 80 and 5 mg/mL, respectively. At specified time points, cell-seeded composites were removed and incubated with TrypLE Express to extract RNA by following the procedure from the Purelink RNA kit (Cat #12183018A, Thermo Fisher Scientific, Waltham, MA, USA). Extracted RNA was evaluated for quantity and purity using a Take3 Micro-Volume plate and Cytation3 spectrophotometer (Biotek, Winooski, VT, USA). cDNA was reverse-transcribed using High-Capacity RNA-to-cDNA kit (Cat #4387406, Thermo Fisher Scientific, Waltham, MA, USA) following the protocol from the manufacturer. The RT2 Profiler PCR Array human fibrosis kit (Cat #PAHS-120-ZE-4, Qiagen, Hilden, Germany) was used to detect the expression of 84 fibrosis-related genes. cDNA (400 ng) made with RT2 first strand kit (Cat #330404, Qiagen, Hilden, Germany) were used with RT2 SYBR Green qPCR Mastermix (Cat #330501, Qiagen, Hilden, Germany) to run qRT-PCR on Bio-Rad CFX 384 real-time system. Data were analyzed by Qiagen RT2 Profiler PCR array data analysis software. GAPDH was used as a housekeeping gene, and AACT values for the genes were analyzed. PCA and hierarchical clustering (HC) were performed and visualized with an open source machine learning software (Orange).

Quantification of H₂O₂ release from CaO₂: The amounts of H₂O₂ released from CaO₂ were quantified using a Quantitative Peroxide Assay Kit (Pierce, Cat #23280) using the ferrous ion (Fe²⁺) oxidation xylenol orange assay[4]. CaO₂ was dissolved in PBS at different pH. CaO₂ was also dissolved in PBS containing catalase (100 U/mL, Fisher Scientific, Cat #S25239A), SLS or TLS (3 mg/mL) at 37° C. An aliquot of 100 μL of working reagent was added to the collected sample of 10 μL and incubated at room temperature for 15 min. The absorbance was measured at 595 nm using Cytation3 spectrophotometer (Biotek, Winooski, VT, USA).

Impact of the presence of CaO₂ on antioxidation capacity of SLS and TLS: The antioxidant activity of SLS and TLS was evaluated using the 2,2-dipehnyl-1-picrylhydrazyl (DPPH, Alfa Aesar, Cat #44150) radical scavenging assay. Briefly, a 0.2 mM DPPH solution was prepared in 1: 1 mixture of ethanol (Fischer Scientific, Cat #BP2818-500, 200 proof) and water (Fisher Scientific, Cat #W2-4) since SLS or TLS is not completely soluble in ethanol. SLS or TLS at 3 mg/mL was dissolved with CaO₂ at concentrations ranging from 0 to 0.5 mg/ml. DPPH solution without sample was used as the control. After incubating samples in darkness at room temperature for 30 min and 24 h with mild agitation of 150 rpm, decreases in absorbance were measured at 517 nm using Cytation3 spectrophotometer (Biotek, Winooski, VT, USA). L-ascorbic acid at 3 mg/ml (Sigma-Aldrich, Cat #-A4403-100 MG) was used as a positive control. Absorbance of all samples without DPPH was subtracted to correct the background absorbance at 517 nm. The DPPH radical scavenging activity (%) was calculated using the following formula: DPPH radical scavenging activity (%)=(A_{c}−Aₛ)/A_{c}×100 (%), where Ac is absorbance of control and As is absorbance of samples.

Intracellular ROS assay: The ROS scavenging capability of TLS was assessed in cultures of C2C12 myocytes. Cultures of C2C12 were maintained by complete medium including 88% DMEM (Corning, Cat #-10-013-CV), 10% FBS (Corning, Cat #-MT35010CV, USDA tested), 1% Penicillin/Streptomycin (Lonza Cat #17-602E) and 1% L-glutamine (Corning Cat #25-005-CI). After treating C2C12 myocytes with H₂O₂ (3% solution in water, Thermo Fisher Scientific, Cat #AC426000250) for 2 h, TLS at 0.1 and 1.0 mg/mL was added in SF C2C12 culture medium for 18 h at 37° C./5% CO2. After washing, C2C12 myocytes were cultured with 10 UM of di(acetoxymethyl ester) analog of DCFDA (Thermo Fisher, Cat #C2938) for 30 min at 37° C./5% CO2, washed and imaged.

### Example 3: Lignin Composites with Sustained Oxygenation and Reactive Oxygen-Species Scavenging Improve Neovascularization and Healing of Diabetic Wounds

### Methods

Synthesis of Lignin Composites: To produce TLS, SLS (TCI Chemicals) was functionalized with 3-mercaptopropionic acid (MPA) in hydrochloric acid. It was confirmed that the thiolation is dominant on aliphatic hydroxide of SLS and the extent of thiolation of TLS was tunable by modulating stoichiometry of MPA. The coupling of lignosulfonate to PLGA (poly(lactic—co-glycolic) acid) was performed by acylation reaction for SLS in a mass ratio of 2 to 1. SLS-graft-PLGA and CaO₂ were dissolved in ethyl acetate (organic phase) under stirring at room temperature. The suspension was homogenized with a microfluidizer (Microfluidics Corp., Westwood, MA) at 30 kpsi at 4° C. Next, the organic solvent was evaporated with a rotary evaporator R-300 (Buchi Corp, New Castle, DE). Finally, trehalose was added (1:1 mass ratio) as a cryoprotectant for SLS:PLGA nanoparticle lyophilization and stored at −20° C. for further characterization and tests.

Precursors of lignin composites were reconstituted in 1 mg/mL LAP (Lithium phenyl-2,4,6-trimethylbenzoylphosphinate) in PBS. The final concentrations were 50 mg/ml gelatin methacrylate (GelMA) with TLS (3 mg/mL) plus either CPOc (4 mg/mL, CPO control-SLS particles formed without CaO₂) or CPO (4 mg/mL, SLS particles formed with CaO₂). Precursors were cast into custom molds or topically applied in db/db mouse wounds followed by UV illumination for 60 s (365 nm) for gelation of the lignin composites in situ.

Assessment of Capillary-like Network Formation of MVECs: Murine dermal microvascular endothelial cells (MVECs) were purchased from Cell Biologics and cultured in T-25 flasks coated with 0.1% gelatin (Boston Bioproducts, Milford, MA). Control MVECs were cultured in Endothelial Cell Medium (Sciencell, Carlsbad, CA) with 5% FBS, while MVECs in the test group were treated with 30 mM dextrose in Endothelial Cell Medium with 5% FBS. MVECs in the test group were cultured in 30 mM dextrose for 5-7 days prior to the tube formation assay. On the night prior to the assay, both control and test MVECs were cultured under the previously stated conditions but with Endothelial Cell Medium containing only 1% FBS for cell starvation. On the day of the assay, 50 μL of lignin composites were cast into a custom PDMS well (8 mm diameter and 1 mm height) fixed on a glass slide to prevent meniscus formation. Four groups of lignin composites were cast, I) GelMA, 2) GelMA+TLS, 3) GelMA+TLS+CPOc, and 4) GelMA+TLS+CPO (abbreviation of lignin composites in Table 3). The precursors of lignin composites were then photopolymerized under UV light for 60 s. The hydrogels were washed with PBS twice. MVECs were seeded onto the lignin composites at 5.0×10⁴ cells per well. The MVECs were then placed in a CO2 incubator (37° C./5% CO₂). A set of randomly selected five phase contrast images were taken per each composite using an inverted fluorescence microscope (Nikon Eclipse Ti2) and NIS Elements Advanced Research Microscope Imaging Software (NIS Elements AR, Nikon) at every 24 h over 96 h. Angiogenesis Analyzer in FIJI software was used to assess the capillary-like network formation with the seven relevant-features of angiogenesis discussed in the results.

Assessment of VEGF and HIF-1α Protein Expression in Cultures of MVECs: Following the imaging for network formation of MVECs, supernatant was collected at every 24 h over 96 h, and protease and phosphatase inhibitors were added to the conditioned medium before freezing at −80° C. Expression levels of murine vascular endothelial growth factor (VEGF) (Quantikine ELISA kit, R&D Systems, Minneapolis, MN, USA) and HIF-1α (SimpleStep ELISA kit, Abeam ab275103, Cambridge MA, USA) were determined in the culture medium using ELISA as per manufacturers' protocols. Optical density was read on a BioTek Gen5™ Microplate Reader and standard curves were generated to determine cytokine concentrations using Imager Software.

In Vivo Model of Diabetic Wound Healing: A mouse model of diabetic wound healing was used to validate that the in vitro results of angiogenic responses of MVECs on lignin composites can be translated to improved healing in in vivo diabetic wound conditions. This model is characterized by a delayed wound healing with reduced neovascularization and increase in ROS and inflammation. All procedures were approved by the Institutional Animal Care and Use Committee. Eight-week-old female, diabetic B6.BKS(D)-Lepd^{dp}/J (db/db) mice were obtained from Jackson Laboratories (Bar Harbor, ME). Two full thickness 6 mm circular excisional wounds were created side-by-side on the bilateral flanks on the dorsum of each animal, leaving the underlying muscle (panniculus carnosus) intact, as previously described. A silicone stent was sutured around the wound with inner diameter of 8 mm to prevent wound contraction of loose skinned murine wounds. A sterile transparent dressing Tegaderm^{3M}, was applied to the skin to cover the wounds and held in place with benzoin tincture. Lignin composites containing 1) Ge!MA+LAP, 2) GelMA+LAP+TLS, 3) GelMA+LAP+TLS+CPOc, and 4) GelMA+LAP+TLS+CPO (n=3, per each group), were injected into one wound immediately after wounding. The PBS treatment on the other-side wound served as an internal control for every animal. The animals were euthanized at days 7 and 14; wound tissues were harvested, fixed, and paraffin embedded.

Immunohistochemistry: Skin tissue was harvested, fixed in 10% neutral buffered formalin and paraffin embedded. 5 μm thick sections were cut and mounted onto superfrost plus slides. Slides were deparaffinized and rehydrated to PBS following standard protocol and immunohistochemistry staining was performed on a DAKO Auto-stainer Link 48 (DakoLink version 4.1, edition 3.1.0.987; Agilent, Santa Clara, CA). Primary antibodies against CD31 for endothelial cells (ab28364; 1:100; Abeam, Cambridge, MA), VEGF (MA5-3208; 1:300, Thermo Fisher Scientific, Waltham, MA), HIF-1α (abl 79483; 1:100; Abeam, Cambridge, MA), CD45 for pan leukocytes (abl0558; 1:5000; Abeam, Cambridge, MA), and F4/80 for pan-macrophages (ab 111101; 1:100: Abeam, Cambridge, MA) were applied followed by detection using an EnVision+System-HRP (DAB) kit (Dako North America, Carpinteria, CA) and hematoxylin counter staining. Histology slides were imaged with Leica DM 2000@ with Leica Application Suite X® version 3.0.4.16529. Percentage of positive cells and capillary lumens per HPF within the granulating wound bed were quantified in 6 random fields distributed along the wound bed.

Quantification of VEGF and HIF-1α in the Wound Homogenates: To determine protein expression of VEGF and HIF-1α in the db/db mice wounds treated with different lignin composites, wounds at day 7 post wounding were harvested. 10-50 mg each of the wound tissues were homogenized in 300 μL of freshly prepared RIPA buffer (Thermo Fisher Scientific 89900, Waltham, MA, USA). The digested samples were centrifuged and only the top supernatant was used in the quantification of expression levels of murine VEGF (Quantikine ELISA kit, R&D Systems, Minneapolis, MN, USA) and HIF-1α (SimpleStep ELISA kit, Abeam ab275103, Cambridge MA, USA) as per manufacturers' protocols. Optical density was read on a BioTek Gen5™ Microplate Reader and standard curves were generated to determine cytokine concentrations using Imager Software.

Statistical Analyses: Statistical comparisons between groups were performed by one-way ANOVA. For multiple comparisons, one-way ANOVA with Tukey's post hoc comparison was carried out. P values at least <0.05 were considered statistically significant. All bar graphs represent mean±standard deviation (SD).

### Results

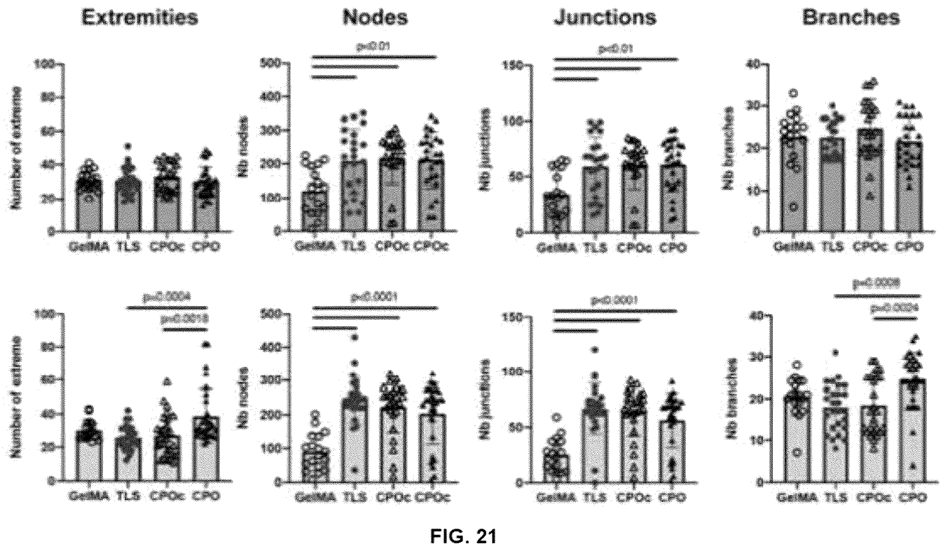

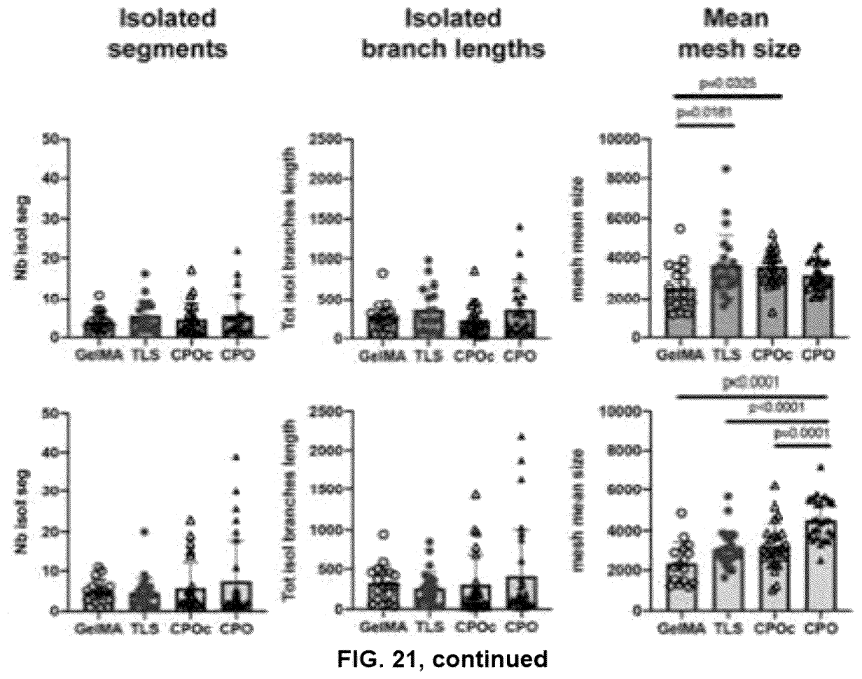

Endothelial capillary-like network formation was improved on dual acting lignin composites: Following 5 days of treatment with 30 mM glucose in 2D cultures, MVECs were seeded onto lignin composites, GelMA, TLS, CPOc and CPO (see Table 3 for details of abbreviations) up to 96 h. Phase contrast micrographs were taken and analyzed using Angiogenesis Analyzer. As shown in FIG. 20, lignin composites supported the growth and capillary-like network formation of MVECs. Seven features were selected from the Angiogenesis Analyzer including number of extremities, number of nodes, number of junctions, number of branches, number of isolated segments, sum of isolated branch lengths, and average mesh sizes (FIG. 21). Lignin composites improved the characteristic capillary-like angiogenesis features in both normal and high glucose (oxidative stress) conditions. These features were significantly higher in CPO lignin composite hydrogels in the presence of high glucose, while those in normal glucose conditions only showed marginal improvements over the GelMA control.

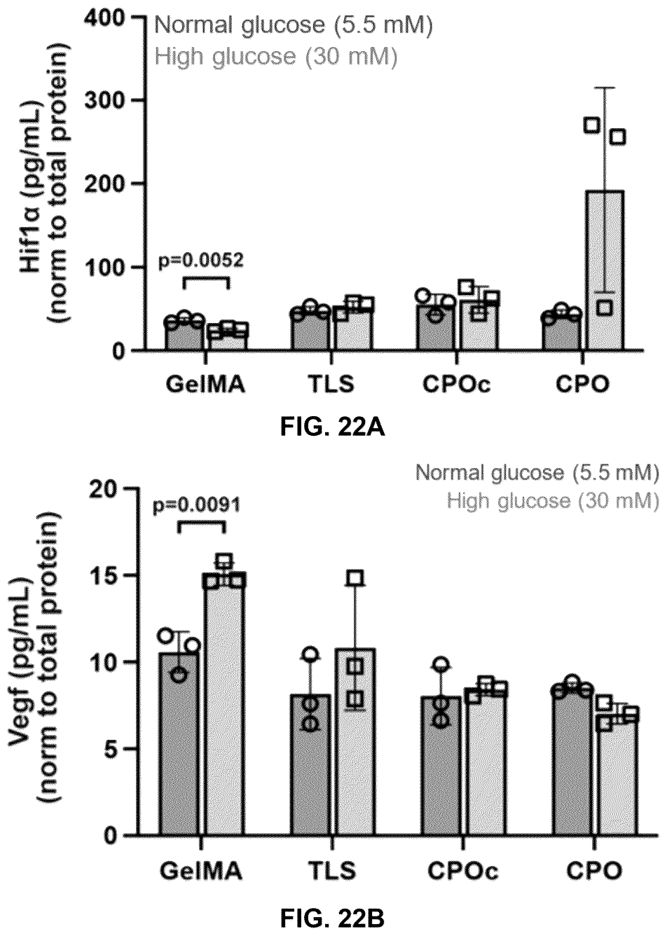

VEGF and HIF-1α expression of MVECs was altered in vitro in lignin composites: VEGF and HIF-1α are two important growth factors involved in neovascularization of the wounds, and they are also highly susceptible to ROS-mediated variations in the wound milieu that ultimately guide angiogenic responses. Hence, it was sought to determine their expression in both control and test group of MVECs cultured in normal and high glucose conditions. HIF-1α was expressed highest in the supernatant of high glucose MVECs on the GelMA (353.4 pg/mL). The MVECs on the TLS, CPOc, and CPO had a downward trend of HIF-1α expression with CPO being the lowest at 243.4 pg/mL. This same trend was present in control MVECs, though with lower values as compared to those under high glucose conditions (GelMA: 276.1 pg/mL, TLS: 272.7 pg/mL, CPOc: 256.2 pg/mL, CPO:248.3 pg/mL) (FIG. 22A). Compared to control cells, VEGF expression increased in MVECs under high glucose conditions. VEGF was expressed highest in the supernatant of high glucose MVECs on the GelMA (30.1 pg/mL). Compared to the GelMA condition, VEGF expression in the supernatant of high glucose MVECs was relatively decreased, though not significantly on TLS (1 7.5 pg/mL), CPOc (19.0 pg/mL), and CPO (16.4 pg/mL). This same trend was seen in the supernatant of control MVECs (FIG. 22B).

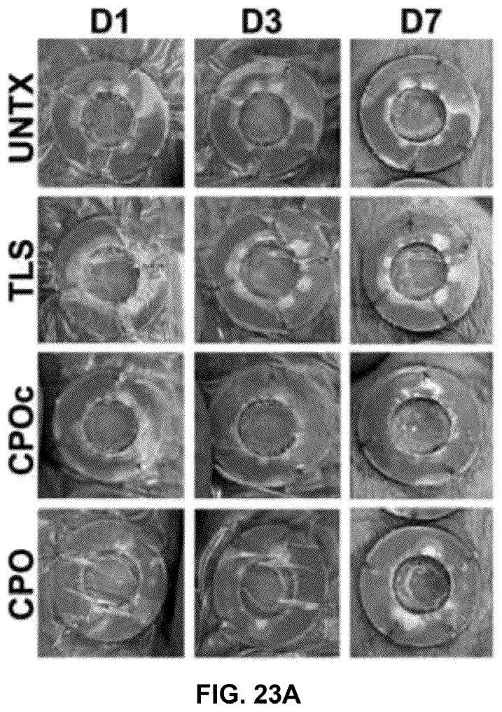

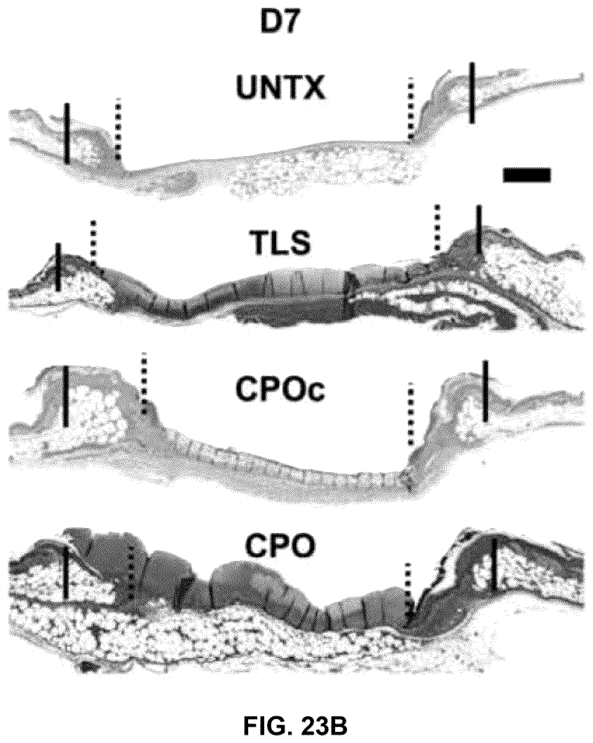

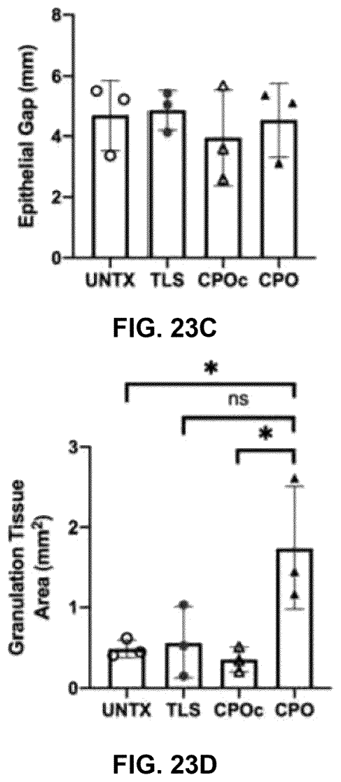

Antioxidant, oxygen-generating dual acting lignin composites improved granulation tissue and capillary lumen formation in db/db wounds at day 7: Wound healing was evaluated in 8-week-old diabetic (db/db) mice with blood glucose >350 mg/dL. 6 mm wounds (FIG. 23A) were treated with lignin composites, TLS, CPO, or CPOc, immediately after wounding on day 0 and their healing was compared to untreated wounds (UNTX). H&E staining of wound sections at day 7 show that there was no difference in epithelial gap amongst all treatment conditions (UNTX: 4.7±1.2 mm, TLS: 4.9±0.4 mm, CPOc: 4.0±1.6 mm, CPO: 4.5±1.2 mm) (FIGS. 23B-23C). However, wounds treated with CPO had more granulating tissue present at day 7 as compared to other treatments (Untreated: 0.5±0.1 mm², TLS: 0.6±0.4 mm², CPOc: 0.4±0.2 mm², CPO: 1.7±0.8 mm²) (FIG. 23D). These data indicate a notable trend with improvement in wound morphology of dual acting, CPO lignin composite-treated wounds. However more animals are needed to achieve better statistical significance.

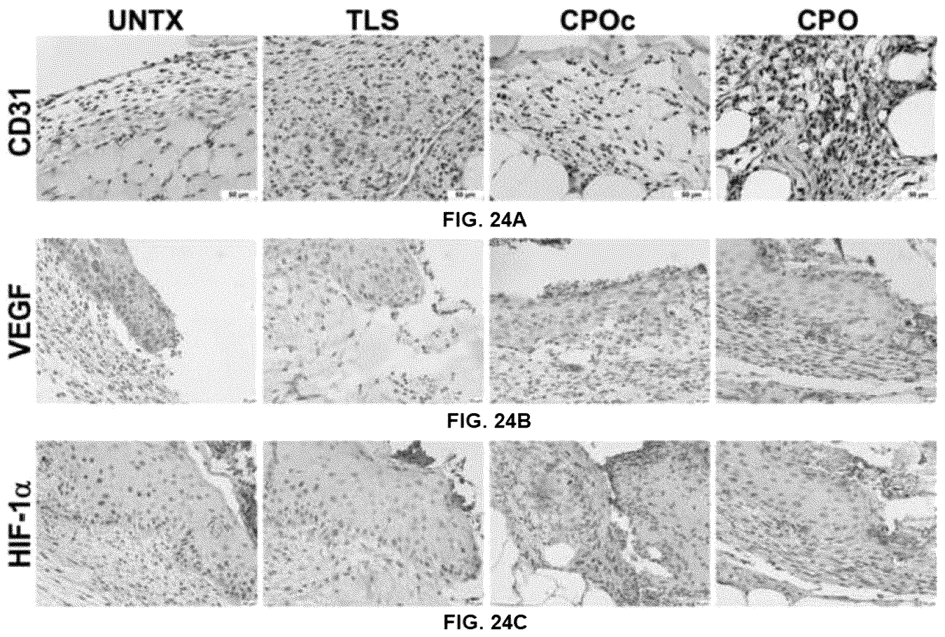

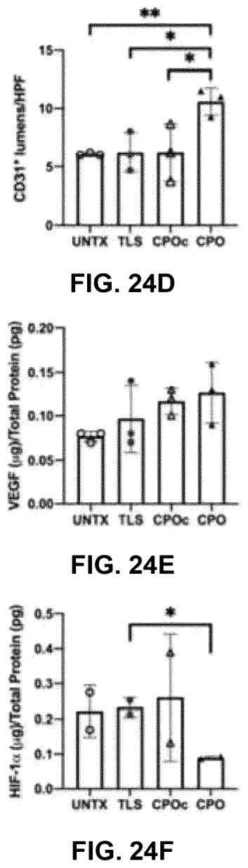

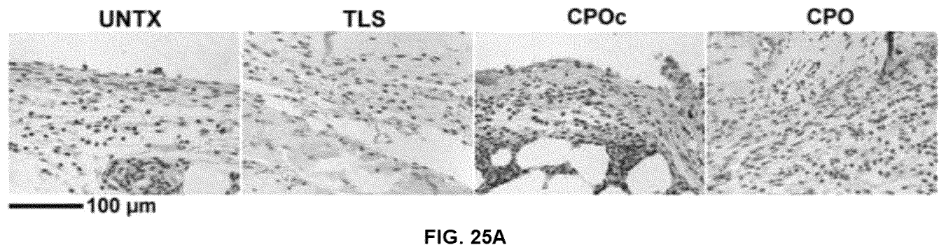

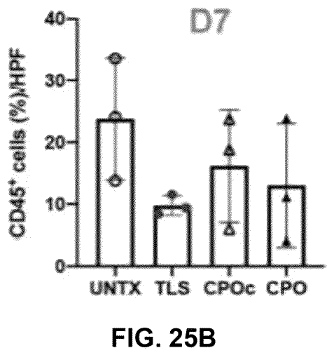

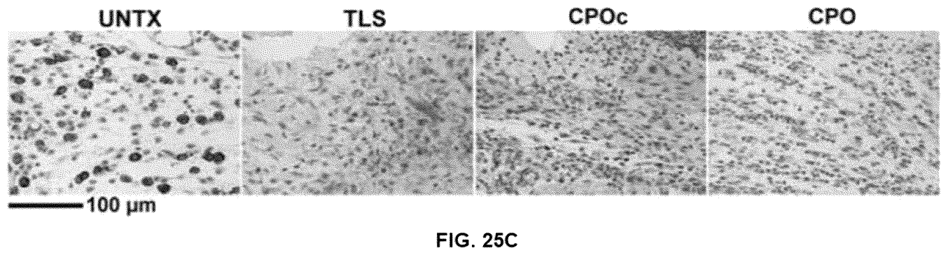

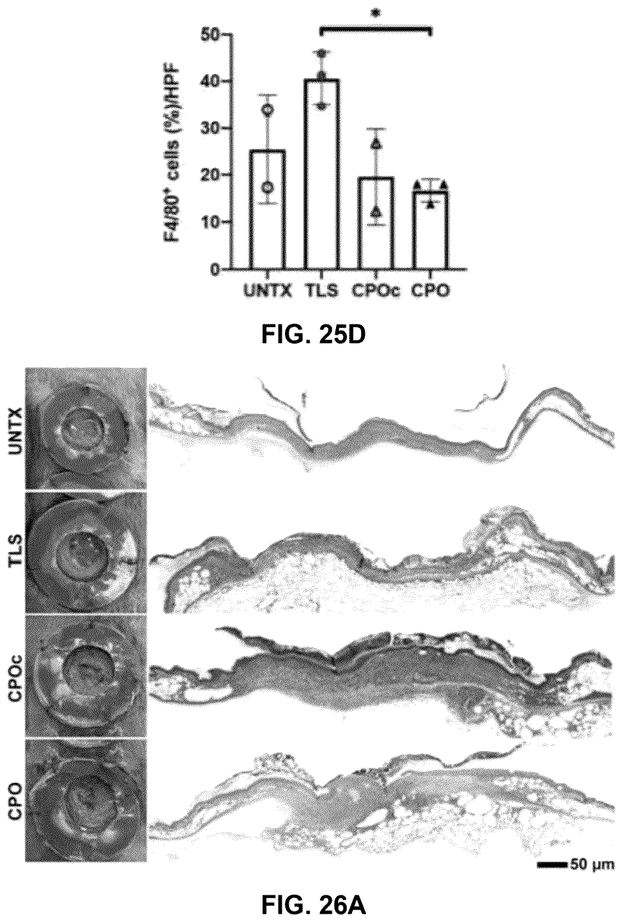

CD31 staining of wound sections collected at day 7 showed a significant increase in capillary lumen density in CPO treated wounds (11±1.2 vessels/HPF), as compared to UNTX (6±0.1 vessels/HPF, p<0.01), TLS (6±1.7 vessels/HPF, p=0.02), and CPOc (6±2.4 vessels/HPF, p=0.049) (FIGS. 24A and 24D). Interestingly, striking differences were noted in VEGF and HIF-1α expression with immunohistochemistry at the leading epidermal margins in CPO vs. other treatments. Consistent with previous literature on normal wound healing in murine skin wounds, db/db mice leading epidermal edges in UNTX wounds displayed abundant VEGF and HIF-1α staining at day 7 post wounding, which is upregulated in response to injury-induced oxidative stress (FIGS. 24B-24C). Similar expression patterns were noted in TLS and CPOc wounds. Strikingly, the oxygen generating lignin composite CPO did not elicit this response at day 7. Histological staining for VEGF and HIF-1α showed reduced expression in the hyperproliferative leading epidermis of CPO-treated wounds at day 7 post wounding. While immunohistochemical staining of VEGF was reduced in the epidermis, quantification of VEGF expression in the homogenized wound tissue using ELISA showed an increase in the VEGF expression at day 7 in the wound bed (FIG. 24E), suggesting dermal angiogenesis is promoted by CPO lignin composites. HIF-1α quantification of the homogenized wounds showed significantly reduced expression in CPO wounds at day 7 as compared to UNTX, TLS, and CPOc wounds (FIG. 24F). In addition, no significant difference was seen in the expression of CD45⁺ (panleukocyte) cells across all tested lignin composites as compared to untreated wounds at day 7. CD45 staining displayed a slight decreased in CPO wounds (13±10% CD45⁺ cells/HPF) compared to untreated (24±10% CD45⁺ cells/HPF, p=0.26), TLS (10±2% CD45⁺ cells/HPF, p=0.60), and CPOc (16±9% CD45⁺ cells/HPF, p=0.72) conditions, although this decrease was not statistically significant (FIGS. 25A and 25C). F4/80 staining displayed a significant decrease in macrophages in CPO-treated wounds compared to TLS-treated wounds (17±2% F4/80⁺ cells/HPF vs. 41±6% F4/80⁺ cells/HPF, p<0.01). F4/80 staining was decreased in CPO treated wounds compared to both untreated (26±12% F4/80⁺ cells/HPF, p=0.26) and CPOc treated (20±10% F4/80⁺ cells/HPF) wounds, however these differences did not reach significance (FIGS. 25B and 25D), indicating minimal inflammatory response from CPO or CPOc nanoparticles in lignin composites.

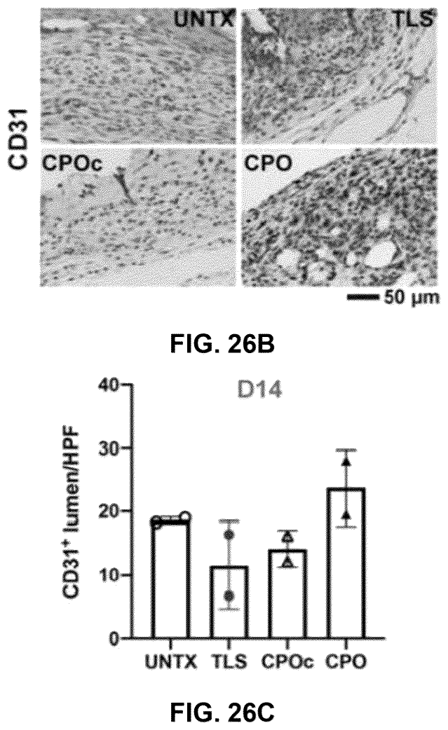

CPO Lignin composites promoted capillary lumen formation and robust granulation tissue remodeling over 14 days in db/db wounds: To determine the effect of the lignin composite treatment on remodeling in db/db wounds, the effect of treatment at additional time point of 14 days post wounding was tested. At day 14, visibly improved healing was noted in CPOc and CPO wounds from gross images, which was supported by the presence of a robust granulating wound bed in representative hematoxylin and eosin-stained wounds sections (FIG. 26A). Day 14 wounds treated with CPO (24±4 CD31⁺ lumens/HPF) also displayed significantly increased lumen density with CD31 staining compared to TLS (12±5 CD31⁺ lumens/HPF) and CPOc (14±2 CD31⁺ lumens/HPF) but did not approach significance compared to UNTX (19±0.33 CD31+lumens/HPF) (FIGS. 26B-26C).

### DISCUSSION

Diabetes is known to activate the ROS system and inactivate antioxidation including enzymes such as superoxide dismutase. This leads to impaired wound healing via impairment of multiple pathways resulting in increased inflammation, decreased granulation tissue formation, and poor blood supply. It was sought to determine whether composites with lignosulfonates would promote diabetic wound healing by rescuing these effects of impaired ROS. Whether antioxidant and oxygen-generating lignin composites can promote endothelial cell functions under hyperglycemic conditions was tested. Murine MVEC from the skin were exposed to high glucose (30 mM) for 5-7 days, and then cultured them on lignin composites of TLS, CPO, CPOc, and GelMA-only control. A significant improvement in EC network formation was noted with CPO (FIGS. 20-21), suggesting that oxygen-generating lignin composites may correct cellular dysfunctions in diabetic endothelial cells. However, recent reports suggest that an in vitro acute hyperglycemic model may not be ideal for mechanistic studies of the effects of diabetes on EC responses, due to a phenomenon called “hyperglycemic memory”. Thus, experimental evidence suggests that oxidative stress results in sustained activation of antiangiogenic, proinflammatory pathways in MVECs even after glycemia is normalized (i.e., the cell phenotype of endothelial cells becomes altered permanently, although the exact mechanisms are not well understood), as shown in previous studies. In addition, previous studies have shown an increase in VEGF expression in diabetic endothelial cells and those under excessive ROS, which was also noted in the cell culture experiments with MVEC under high concentration of glucose. Culture of these cells on the CPO lignin composites reversed these effects induced by high concentration of glucose. The important implication of these findings is that there is a need for novel therapies that can reverse hyperglycemic memory of endothelial cells to promote their cell-cell and cell-matrix interactions and neovascularization, a gap that could be filled with the lignin composite design.

We then harvested wounds from 8-week-old db/db mice with 6 mm wounds treated with lignin composites containing TLS, CPOc, or CPO. It was found that the leading epidermal edges of all wounds in untreated, TLS- and CPOc-treatment exhibited HIF-1α induction, which is expected. However, when there were oxygen generating particles, as with CPO, a significant upregulation of HIF-1α was not observed at the edges, though granulation tissue improvement was seen, signifying that the wound is indeed healing. This suggests that exogenous oxygen released into the wound alters hypoxia-induced pathways in diabetic wounds in a way that reduces HIF-1α expression at wound edges. Further understanding of these pathways leading to HIF-1α expression in diabetic mice treated with lignin composites will be the focus of future projects.

VEGF, which is well-known for its proangiogenic abilities, also plays a significant role in cutaneous wound healing and has also been shown to be involved in hair growth, skin diseases, and cutaneous cancers. Interestingly, despite the known connection between HIF-1α and VEGF, it is seen that VEGF expression is increased at the wound edges of CPO-treated wounds, suggesting that CPO scavenges ROS in the wound and increases VEGF via pathways independent of HIF-1α. The mechanism by which this occurs is not well described and could be a future area of study, however, increased oxygen levels in diabetic wounds have previously been shown to increase wound VEGF levels. In a rat wound model, hyperbaric oxygen treatment resulted in increased VEGF in wound fluid. It has been shown that this increase in VEGF in CPO-treated wounds is accompanied by expected increase in capillary vessel density and improved wound healing. Further understanding of the role of the CPO lignin composites in the regulation of VEGF expression will help improve the design of these scaffolds to increase their wound healing efficacy.

Diabetic wounds are plagued by decreased granulation tissue formation, however, it was seen that granulation tissue was significantly improved at days 7 and 14 in the db/db mouse wounds with CPO. This robust granulation tissue formation in the CPO lignin composites suggests that scavenging the free radicals and ROS via lignin in the wound rescues the alterations caused by hyperglycemic memory and restores robust tissue formation. The mechanism by which this occurs is not yet known, and it is an area of further research. Understanding of the pathways to this restoration of robust granulation tissue may give insight to improve diabetic wounds and decrease the incidence of wound recurrence. There are several limitations to this study. The db/db mouse was used as a model for diabetic wounds. The excisional wounds were made manually rather than spontaneously as in diabetic wounds/ulcers on human skin. Additionally, contraction plays a significant role in healing of excisional wounds in the mouse model, in contrast to human skin, requiring stent placement to limit this effect as previously described. Pig models are a superior model for studying wound healing, as the skin architecture is akin to that of humans; thus, pig models will be used in future studies.

### Conclusions

We have shown that ROS scavenging by the constitutes of lignin composites along with locoregional oxygen release decreases HIF-1α expression, increases VEGF expression, and increases granulation tissue formation in a wounded db/db mouse model. These data support the well-known notion that oxidative stress causes impaired wound healing and suggest an engineered solution for improving diabetic wound healing by increasing oxygen levels in the wound while simultaneously scavenging free radicals using the CPO lignin composite. Future studies will be focused on data-driven optimization of the CPO lignin composite to further improve the diabetic wound microenvironment with the goal of transition to large animal studies and translation to human diabetic wounds.

### Example 4: Additional Modifications of TLS to Confer Antimicrobial Properties

FIG. 27 shows a scheme for adding amino groups to thiolated lignosulfonate. A thiolated lignosulfonate (prepared as described above), hexamethylenediamine, formaldehyde, and 5% NaOH are admixed and held for 1 h in an ice bath to provide amino group-containing substituents as shown (i.e., aminoalkyl TLS). The progress of this reaction was monitored by ¹H NMR (FIG. 28).

This functionalization is driven by the enhanced antimicrobial properties of aminoalkyl kraft lignin, which have been demonstrated to be even more effective against S. aureus and E. coli than copper ions. Therefore, these functional groups will be applied to the presently disclosed TLS compounds and compositions, creating dual-functioning lignosulfonate nanoparticles.

Additional functionalization of TLS to confer antimicrobial properties: This functionalization is driven by the enhanced antimicrobial properties of aminoalkyl kraft lignin, which have been demonstrated to be even more effective against S. aureus and E. coli than copper ions. Therefore, these functional groups were applied to TLS, creating dual-functioning lignosulfonate nanoparticles.

Expression of HIF1α and VEGF can be modulated by CPO composites in cultures of MVECs: HIF1α stimulates angiogenesis while VEGF promotes the initial capillary sprouting and angiogenic responses during normal wound repair, both of which are abnormally altered in diabetic conditions. Thus, the expression of HIF1α and VEGF in cultures of mouse MVECs on lignosulfonate composites was assessed. High glucose levels reduced HIF1α without lignosulfonate (GelMA intracomparison, FIG. 22A), but slightly increased in TLS and CPOc (intercomparison to GelMA, FIG. 22A). Notably, in CPO composites, a 5-fold increase in HIF1α was noted under high glucose, indicative of the protective mechanisms for glucose impairment in HIF1α stabilization. Secretion of VEGF showed different patterns. High glucose MVECs produced more VEGF than normal glucose levels, with GelMA control being most pronounced (FIG. 22B). This was in accordance with other studies that showed increased VEGF expression by diabetic ECs. CPO composites reduced this high glucose induced upregulation of VEGF. This indicates that the CPO composites can correct the altered VEGF overexpression in diabetes. HIF1α and VEGF are known to have dose- and time-dependent affects, where inadequate or deficient expression can lead to non-healing chronic wounds, while overexpression can promote leaky vasculature, excessive scarring or fibrosis.

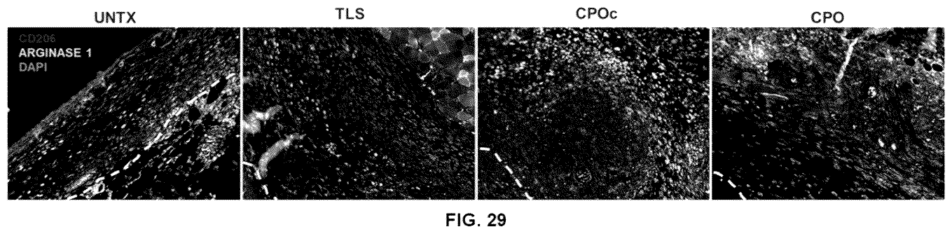

Reduced inflammatory response in diabetic wounds by CPO composites (in vivo): More macrophages in the treatment with CPO composites were immunoreactive for CD206 and arginase 1, indicating that they may be of a pro-healing phenotype (FIG. 29).

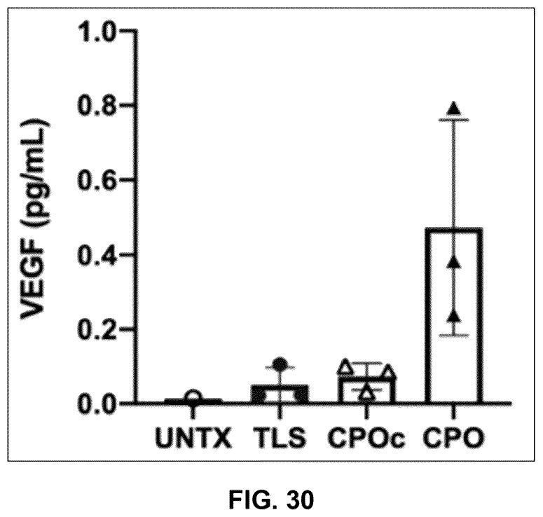

Dermal angiogenesis is promoted by the CPO composites: VEGF expression in the homogenized wound tissue showed an increase in the levels of VEGF at day 7 using immunoblotting with ProteinSimple WES (FIG. 30).

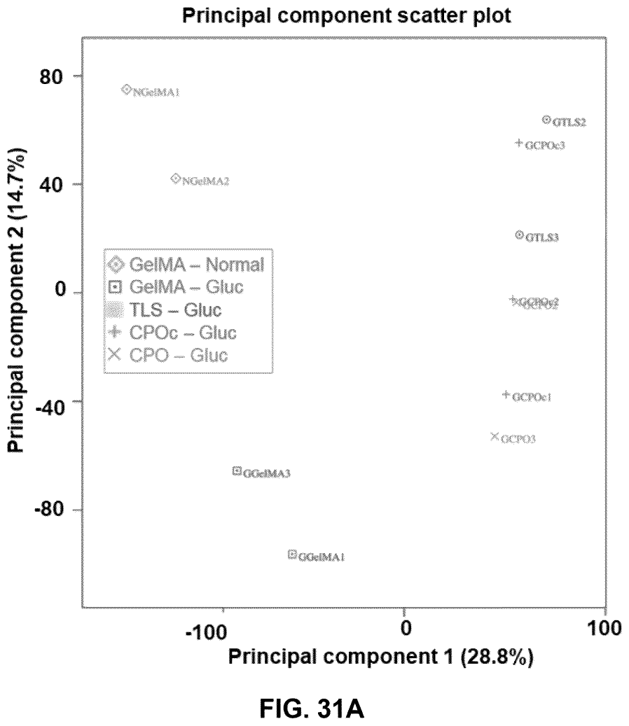

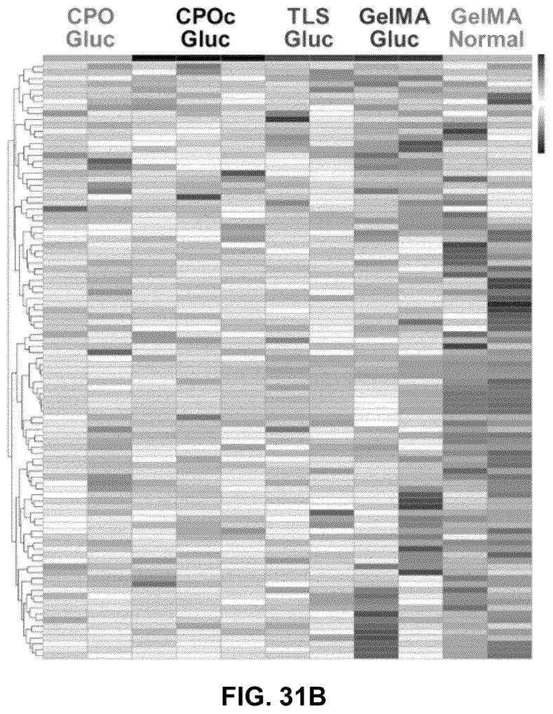

RNA sequencing of mouse MVECs cultured on lignosulfonate composites in normal and hyperglycemic conditions: Preliminary data showed a significant improvement in MVEC network formation with CPO composites in both normal and high-glucose induced oxidative stress conditions. To determine the mechanisms by which the lignosulfonate composites influence MVEC phenotype, bulk RNA sequencing was performed on MVECs cultured on lignosulfonate composites for 4 days under high-glucose (30 mM) or normal-glucose (control, 5.5 mM) conditions. RNA was extracted (PureLink RNA Mini kit) and bulk RNA sequencing was conducted on Illumina NextSeq2000. Samples underwent a second round of sequencing in order to achieve the desired sequencing coverage. QC and Count analyses (Expression Browser) were performed and samples with ˜50000 reads were included in further data analysis. PCA (principal component analysis) showed a clear separation of EC gene expression on GelMA (no lignosulfonate treatment) in normal culture from high glucose conditions, and EC gene expression on TLS (antioxidation only) from CPOc (oxygenation vehicle only) and CPO (oxygenation enabled) in high glucose conditions (FIG. 31A). Differential gene expression (DGE) was compared, and the heatmap shows the top 100 DGE between ECs across the entire sample set (FIG. 31B). There are clear differences in EC genes among GelMA Normal vs. GelMA Glucose controls. Several genes from ECs under high glucose conditions cultured on the radical scavenging, oxygen releasing CPO lignosulfonate composite appear drastically different from GelMA-Glucose controls (FIG. 31B).

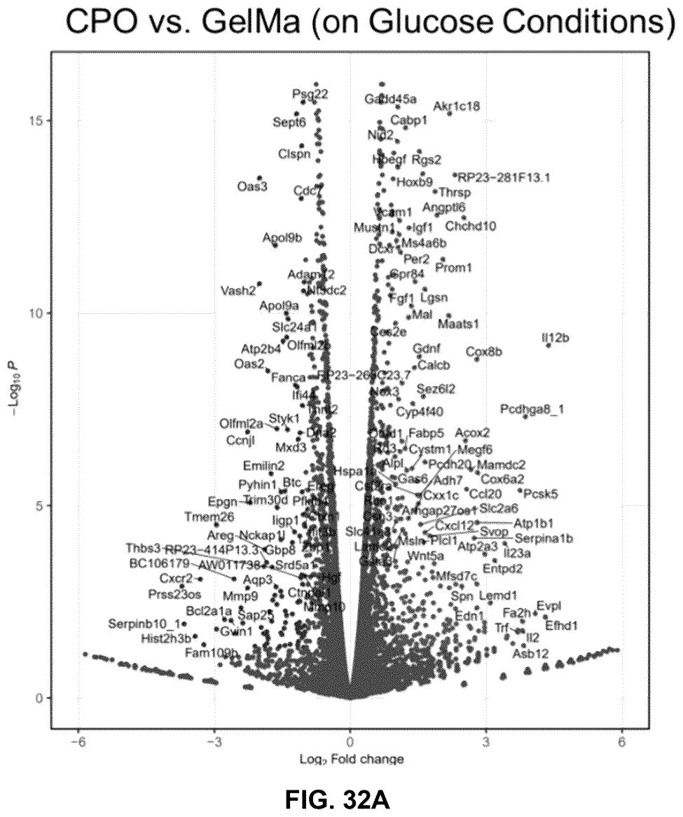

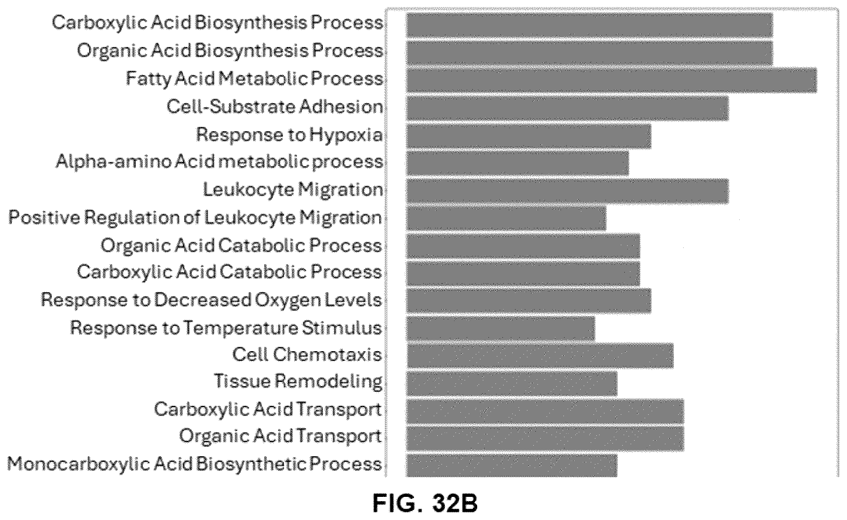

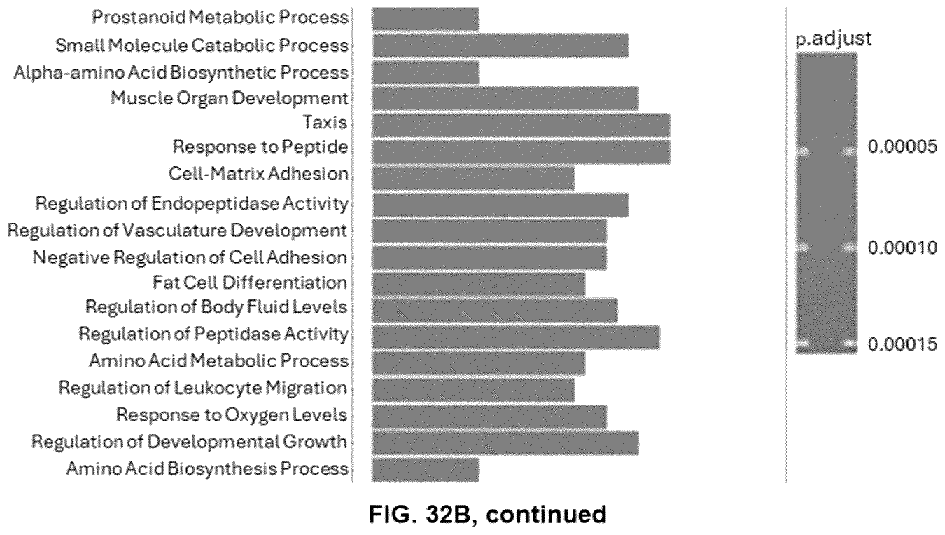

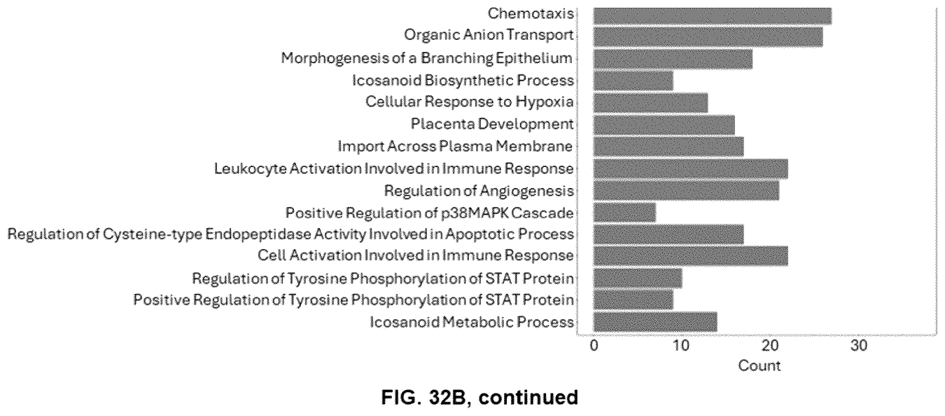

Significantly up/downregulated groups were then plotted using volcano plots. Comparison of ECs cultured on GelMA Glucose control vs. CPO lignosulfonate composite under high glucose conditions highlighted several genes that were different. Volcano plots highlight genes that are fold change >2 and p<0.05 (FIG. 32A), and Gene Ontology (GO) analyses was also performed to determine enriched biological process (BP) (FIG. 32B), which highlight hypoxia inducible, angiogenesis and vascular maturation genes and pathways influenced by CPO lignosulfonate composites.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Alemdar, N.; et al, Oxygen-Generating Photo-Cross-Linkable Hydrogels Support Cardiac Progenitor Cell Survival by Reducing Hypoxia-Induced Necrosis. ACS Biomater. Sci. Eng. 2017, 3 (9), 1964-1971.
2. Aro, T. et al., 2017, “Production and Application of Lignosulfonates and Sulfonated Lignin,” ChemSusChem, 10:1861-1877.
3. Ashammakhi, N.; et al, Advances in Controlled Oxygen Generating Biomaterials for Tissue Engineering and Regenerative Therapy. Biomacromolecules 2020, 21 (1), 56-72.
4. Astete. C. E.; et al. Lignin-Graft-Poly(lactic-co-glycolic) Acid Biopolymers tor Polymeric Nanoparticle Synthesis. ACS Omega 2020. 5 (17), 9892-9902.
5. Baba, S. A. et al, Determination of total phenolic and flavonoid content, antimicrobial and antioxidant activity of a root extract of Arisaema jacquemontil Blume. Journal of Taibah University for Science, 2015. 9(4): p. 449-454.
6. Balaji, S., et al., Engineering Antioxidant and Oxygen-Releasing Lignin Composites to Accelerate Wound Healing. bioRxiv, 2022: p. 2022.03.18.484913.
7. Balaji, S., et al., Tissue-engineered provisional matrix as a novel approach to enhance diabetic wound healing. Wound Repair Regen, 2012. 20(1): p. 15-27.
8. Bao, D., et al. Self-assembly of a renewable lignin biopolymer in nanoparticles with multiple morphologies. Journal of Applied Polymer Science 136, 47482 (2019).
9. Belgodere, J. A.; et al, Attenuating Fibrotic Markers of Patient-Derived Dermal Fibroblasts by Thiolated Lignin Composites. ACS Biomater. Sci. Eng. 2021, 7 (6), 2212-2218.
10. Belgodere, J. A.; et al, Modulating Mechanical Properties of Collagen-Lignin Composites. ACS Appl. Bio. Mater. 2019, 2 (8), 3562-3572.
11. Belgodere, J. A. 2020, “Incorporation of Lignin in Natural and Synthetic Biomaterials to Alter Mechanical and Biochemical Properties for Enhanced Wound Healing,” Doctoral Dissertation, Department of Biological and Agricultural Engineering, Louisiana State University and Agricultural and Mechanical College.
12. Blatt, S. E.; et al, Characterizing the Macrophage Response to Immunomodulatory Biomaterials Through Gene Set Analyses. Tissue Eng. Part C Methods 2020, 26 (3), 156-169.
13. Botusan, I. R., et al., Stabilization of HIF-1alpha is critical to improve wound healing in diabetic mice. Proc Natl Acad Sci USA, 2008. 105(49): p. 19426-31.
14. Boulton, A. J. M., et al., in Diagnosis and Management of Diabetic Foot Infections. 2020, American Diabetes Association: Arlington (VA).
15. Callahan, L. A. et al. ECM production of primary human and bovine chondrocytes in hybrid PEG hydrogels containing type I collagen and hyaluronic acid. Biomacromolecules 13, 1625-1631, doi:10.1021/bm3003336 (2012).
16. Camci-Unal, G. ; et al, Hydrogels for Cardiac Tissue Engineering. NPG Asia Mater. 2014, 6 (5). e99-e99.
17. Cano Sanchez, M., et al., Targeting Oxidative Stress and Mitochondrial Dysfunction in the Treatment of Impaired Wound Healing: A Systematic Review. Antioxidants (Basel), 2018. 7(8).
18. Carey, A. V., et al. Expression of alpha-smooth muscle actin in the developing kidney vasculature. Hypertension 19, 11168-175, doi:10.1161/01.hyp.19.2_suppl.ii168 (1992).
19. Carpentier, G., et al., Angiogenesis Analyzer/or ImageJ-A comparative morphometric analysis of “Endothelial Tube Formation Assay” and “Fibrin Bead Assay”. Sci Rep, 2020. 10(1): p. 11568.
20. Chevalier, L. et al. Feasibility of calcium peroxide as an oxygen releasing compound in treatment walls. International Journal of Environment and Waste Management 2, 245-256 (2008).
21. Cho. H., et al., Diabetic wound healing in a MMP9-1-mouse model. Wound Repair Regen, 2016. 24(5): p. 829-840.
22. Cho, H., et al., Regulation of endothelial cell activation and angiogenesis by injectable peptide nanofibers. Acta Biomater, 201 2. 8(1): p. 154-64.
23. Choe. G., et al., Anti-oxidant activity reinforced reduced graphene oxide/alginate microgels: Mesenchymal stem cell encapsulation and regeneration of infarcted hearts. Biomaterials, 201 9. 225: p. 119513.
24. Costantino, S., et al, Hyperglycemia: a bad signature on the vascular system. Cardiovasc Diagn Ther, 2015. 5(5): p. 403-6.
25. Dai J; et al, Advanced Glycation End Products (AGEs) Induce Apoptosis of Fibroblasts by Activation ofNLRP3 Intlammasome via Reactive Oxygen Species (ROS) Signaling Pathway. Med. Sci. Monit. 2019, 25, 7499-7508.
26. Das, A., et al., Enguliment of apoptotic cells by macrophages: a role of microRNA-21 in the resolution of wound inflammation. J Immunol, 2014. 192(3): p. 1120-9.
27. Das. B., et al., In Vivo Cell Tracking, Reactive Oxygen Species Scavenging, and Antioxidative Gene Down Regulation by Long-Term Exposure of Biomass-Derived Carbon Dots. ACS Biomaterials Science & Engineering, 2019. 5(1): p. 346-356.
28. Dean, J. C., et al. Ultraviolet spectroscopy of fundamental lignin subunits: guaiacol, 4-methylguaiacol, syringol, and 4-methylsyringol. J Chem Phys 139, 144313, doi:10.1063/1.4824019 (2013).
29. Delamaire, M., et al., Impaired leucocyte functions in diabetic patients. Di abet Med. 1997. 14(1): p. 29-34.
30. Demsar, J. et al. Orange: data mining toolbox in Python. the Journal of machine Leaming research 14, 2349-2353 (2013).
31. Deoli, N. T.; et al, Status of the Louisiana Accelerator Center. AIP Conf Proc. 2019, 2160 (1). 040005.
32. Deptula, P.; et al, Tissue Rheology as a Possible Complementary Procedure to Advance Histological Diagnosis of Colon Cancer. ACS Biomater. Sci. Eng. 2020, 6 (10), 5620-5631.
33. Detoraki, A., et al., Vascular endothelial growth factors synthesized by human lung mast cells exert angiogenic effects. J Allergy Clin Immunol, 2009. 123(5): p. 1142-9.
34. Dizhbite, T., et al., Characterization of the radical scavenging activity of lignins—natural antioxidants. Bioresour Technol, 2004, 95(3): p. 309-17.
35. Dominguez-Robles, J. et al. Antioxidant PLA Composites Containing Lignin for 3D Printing Applications: A Potential Material for Healthcare Applications. Pharmaceutics 11, doi:10.3390/pharmaceutics11040165 (2019).
36. Dou, Z. et al., 2022, “One-step preparation of lignin-based solid acid by carbonization and sulfonation to catalyze the synthesis of dimethyl adipate,” Industrial Crops and Products, 180:114721.
37. Dunnill, C., et al., Reactive oxygen species (ROS) and wound healing: the functional role of ROS and emerging ROS-modulating technologies for augmentation of the healing process. Int Wound J, 2017. 14(1): p. 89-96.
38. Dworzanski, J., et al., Glutathione peroxidase (GPx) and superoxide dismutase (SOD) activity in patients with diabetes mellitus type 2 infected with Epstein-Barr virus. PLOS One, 2020. 15(3): p. e0230374.
39. Eastoe, J. E., The Amino Acid Composition of Mammalian Collagen and Gelatin. The Biochem. J., 1955. 61 (4): p. 589-600.
40. Erdem, A. et al. 30 Bioprinting of Oxygenated Cell-Laden Gelatin Methacryloyl Constructs. Advanced Healthcare Materials 9, 1901794, (2020).
41. Evrosimovska, B., Dimova, C., Kovacevska, I. & Panov, S. Concentration of collagenases (MMP-1,-8,-13) in patients with chronically inflamed dental pulp tissue. Prilozi 33, 191-204 (2012).
42. Falanga, V., Wound healing and its impairment in the diabetic loot. Lancet, 2005. 366(9498): p. 1736-43.
43. Farris, A. L.; et al, Oxygen Delivering Biomaterials for Tissue Engineering. J. Mater. Chem. B 2016, 4 (20), 3422-3432.
44. Figueiredo, P. et al. In vitro evaluation of biodegradable lignin-based nanoparticles for drug delivery and enhanced antiproliferation effect in cancer cells. Biomaterials 121, 97-108, doi:10.1016/j.biomaterials.2016.12.034 (2017).
45. Folkman, J., et al., Isolation of a tumor factor responsible for angiogenesis. J Exp Med, 1971. 133(2): p. 275-88.
46. Forsythe, J. A., et al., Activation of vascular endothelial growth factor gene transcription by hypoxia-inducible factor J. Mol Cell Biol, 1996. 16(9): p. 4604-13.
47. Fu, X. et al. Specialized fibroblast differentiated states underlie scar formation in the infarcted mouse heart. J Clin Invest 128, 2127-2143, doi:10.1172/jci98215 (2018).
48. Gabov, K., et al. Preparation, characterization and antimicrobial application of hybrid cellulose-lignin beads. Cellulose 24, 641-658 (2017).
49. Gan, D.; et al, Plant-Inspired Adhesive and Tough Hydrogel Based on Ag-Lignin Nanoparticles-Triggered Dynamic Redox Catechol Chemistry. Nat. Comm. 2019, JO (1), 1487.
50. Genovese, A., et al., Angiogenesis, lymphangiogenesis and atopic dermatitis. Chem Immunol Allergy. 2012. 96: p. 50-60.
51. Glovaci, D., et al, Epidemiology of Diabetes Mellitus and Cardiovascular Disease. Curr Cardiol Rep. 2019. 21 (4): p. 2 1.
52. Goldin, A., et al., Advanced glycation end products: sparking the development of diabetic vascular injury. Circulation, 2006. 114(6): p. 597-605.
53. Gonzalez-Pacheco, F. R., et al., Mechanisms of endothelial response to oxidative aggression: protective role of autologous VEGF and induction of VEGFR2 by H202. Am J Physiol Heart Circ Physiol, 2006. 291(3): p. HI 395-401.
54. Gordts, S. C., et al., The Low-Cost Compound Lignosulfonic Acid (LA) Exhibits Broad-Spectrum Anti-HIV and Anti-HSV Activity and Has Potential lor Microbicidal Applications. PLOS ONE, 2015. 10(7): p. e0 131219.
55. Graney, P. L.; et al, In vitro Response of Macrophages to Ceramic Scaffolds Used for Bone Regeneration. J. R. Soc. Inteiface 2016, 13 (120).
56. Gregorova, A., et al. in Proceedings, NANOCON 3rd Int. Cont. 184-189 (2011).
57. Hajihashemi, S., et al, Antioxidant potential in Stevia rebaudiana callus in response to poly ethylene glycol, paclobutrazol and gibberellin treatments. Physiol Mol Biol Plants, 2018. 24(2): p. 335-341.
58. Hamilton, N. B.; et al, Pericyte-Mediated Regulation of Capillary Diameter: A Component ofNeurovascular Coupling in Health and Disease. Front. Neuroenergetics 2010, 2, 5.
59. Han, X.; et al, Metformin Accelerates Wound Healing In Type 2 Diabetic db/db Mice. Mo !. Med. Rep. 2017, 16 (6), 8691-8698.
60. Harrison, B. S., et al., Oxygen producing biomaterials for tissue regeneration. Biomaterials. 2007. 28(31): p. 4628-34.
61. Hasegawa, Y., et al., Lignosulfonic acid promotes hypertrophy in 3T3-LI cells without increasing lipid content and increases their 2-deoxyglucose uptake. Asian-Australas J Anim Sci, 2017. 30(1): p. 111-118.
62. Hassan, S.; et al, Survival and Proliferation under Severely Hypoxic Microenvironments Using Cell-Laden Oxygenating Hydrogels. J Funct. Biomater. 2021, 12 (2).
63. He, Z.; et al Reactive Oxygen Species (ROS): Utilizing Injectable Antioxidative Hydrogels and ROS-Producing Therapies to Manage the Double-Edged Sword. J Mater. Chem. B 2021, 9 (32), 6326-6346.
64. Heble, A. Y., et al. Catalase-Loaded Silica Nanoparticles Formulated via Direct Surface Modification as Potential Oxygen Generators for Hypoxia Relief. ACS Appl Mater Interfaces 13, 5945-5954, doi:10.1021/acsami.0c19633 (2021).
65. Hong, J. H., et al. A simple morphometric analysis method for dermal microstructure using color thresholding and moments. Skin Res Technol 26, 132-136, doi:10.1111/srt. 12776 (2020).
66. Hong, W. X., et al., The Role of Hypoxia-Inducible Factor in Wound Healing. Adv Wound Care (New Rochelle), 2014. 3(5): p. 390-399.
67. Hosgood, G., Wound healing. The role of platelet-derived growth factor and transforming growth factor beta. Vet Surg, 1993. 22(6): p. 490-5.
68. Hsieh, T. E. et al. Optimizing an Injectable Composite Oxygen-Generating System for Relieving Tissue Hypoxia. Front Bioeng Biotechnol 8, 511, doi:10.3389/fbioe.2020.00511 (2020).
69. Huang, C., et al., Unveiling the Structural Properties of Lignin-Carbohydrate Complexes in Bamboo Residues and Its Functionality as Antioxidants and Immuno-stimulants. ACS Sustainable Chemistry & Engineering, 2018. 6(9): p. 12522-12531.
70. Huang, H.; et al, Co-transplantation of Islets-Laden Microgels and Biodegradable O(2)-Generating Microspheres for Diabetes Treatment. ACS Appl. Mater. Interfaces 2022, 14 (34), 38448-38458.
71. Hunt, T. K., et al., Anaerobic metabolism and wound healing: an hypothesis for the initiation and cessation of collagen synthesis in wounds. Am J Surg, 1978. 135(3): p. 328-32.
72. Hurley, J. R., et al., Nanofiber Microenvironment Effectively Restores Angiogenic Potential of Diabetic Endothelial Cells. Adv Wound Care (New Rochelle), 2014. 3(11): p. 7 17-728.
73. luchi, Y.; et al, Spontaneous Skin Damage and Delayed Wound Healing in SODI-Deficient Mice. Mol. Cell. Biochem. 2010, 341 (1-2), 181-94.
74. Jaiswal, L., et al., Lignin-mediated green synthesis of AgNPs in carrageenan matrix/or wound dressing applications. Int J Biol Macromol, 2020. 159: p. 859-869.
75. Javed, R., et al., PVP and PEG doped CuO nanoparticles are more biologically active: Antibacterial, antioxidant, antidiabetic and cytotoxic perspective. Mater Sci Eng C Mater Biol Appl. 2017. 79: p. 108-115.
76. Jin, G., et al., mRNAs expression profiles of high glucose-induced memory in human umbilical vein endothelial cells. Diabetes Metab Syndr Obes, 2019. 12: p. 1249-1261.
77. Johnson, K. E. et al, Vascular Endothelial Growth Factor and Angiogenesis in the Regulation of Cutaneous Wound Repair. Adv Wound Care (New Rochelle), 2014. 3(10): p. 647-661.
78. Joshi Navare, K. et al. Needle-injectable microcomposite cryogel scaffolds with antimicrobial properties. Scientific Reports 10, 18370, doi:10.1038/s41598-020-75196-1 (2020).
79. Kai, D.; et al, Sustainable and Antioxidant Lignin-Polyester Copolymers and Nanofibers for Potential Healthcare Applications. ACS Sustain. Chem. Eng. 2017, 5 (7), 6016-6025.
80. Kai, D. et al. Development of lignin supramolecular hydrogels with mechanically responsive and self-healing properties. ACS Sustainable Chemistry & Engineering 3, 2160-2169 (2015).
81. Kai, D., et al. Engineering highly stretchable lignin-based electrospun nanofibers for potential biomedical applications. J Mater Chem B 3, 6194-6204, doi: 10.1039/c5tb00765h (2015).
82. Kai, D., et al., Engineering Poly(Lactide). Lignin Nano.fibers with Antioxidant Activity for Biomedical Application. ACS Sustainable Chem. Eng., 2016. 4(10): p. 5268.
83. Kang, J. I.; et al, Advances in Gelatin-Based Hydrogels for Wound Management. J Mater. Chem. B 2021, 9 (6), 1503-1520.
84. Kedare, S. B. et al, Genesis and development of DPPH method of antioxidant assay. J Food Sci Technol, 2011. 48(4): p. 412-22.
85. Keswani, S. G., et al., Adenoviral mediated gene transfer of PDGF-B enhances wound healing in type I and type II diabetic wounds. Wound Repair Regen, 2004. 12(5): p. 497-504.
86. Khorshidi, S.; et al, Fabrication of Amine-Decorated Nonspherical Microparticles with Calcium Peroxide Cargo for Controlled Release of Oxygen. J. Biomed. Maler. Res. A 2020, 108 (1), 136-147.
87. Kim, J. H., et al., High Levels of Oxidative Stress and Skin Microbiome are Critical for Initiation and Development of Chronic Wounds in Diabetic Mice. Sci Rep, 201 9. 9(1): p. 19318.
88. Kim. S. Y.; et al, Macrophages in Wound Healing: Activation and Plasticity. Immunol. Cell Biol. 2019, 97 (3), 258-267.
89. Lan, C. C., et al., High-glucose environment enhanced oxidative stress and increased interleukin-8 secretion from keratinocytes: new insights into impaired diabetic wound healing. Diabetes, 2013. 62(7): p. 2530-8.
90. Larcher, F., et al., VEGFIVPF overexpression in skin of transgenic mice induces angiogenesis, vascular hyperpermeability and accelerated tumor development.

Oncogene, 1998. 17(3): p. 303-11.
91. Lee, J. G. et al. Lignin-Zein Composite: Synthesis, Three-Dimensional Printing, and Microbial Degradation. ACS Sustainable Chemistry & Engineering 9, 1781-1789, doi:10.1021/acssuschemeng.0c07915 (2021).
92. Lee. J., et al., Inhibition of Hippo Signaling Improves Skin Lesions in a Rosacea-Like Mouse Model. Int J Mol Sci, 202 1. 22(2).
93. Lee. M. H., et al., Lignin-based barrier restricts pathogens to the infection site and confers resistance in plants. Embo j. 2019. 38(23): p. e101948.
94. Lee, Y. et al. In Situ Forming and H(2)O(2)-Releasing Hydrogels for Treatment of Drug-Resistant Bacterial Infections. ACS Appl Mater Interfaces 9, 16890-16899, doi:10.1021/acsami.7b03870 (2017).
95. Leung, D. W., et al., Vascular endothelial growth factor is a secreted angiogenic mitogen. Science, 1989. 246(4935): p. 1306-9.
96. Levy, A. P., et al., Transcriptional regulation of the rat vascular endothelial growth factor gene by hypoxia. J Biol Chem, 1995. 270(22): p. 13333-40.
97. Lewis, M. C.; et al, Heterogeneous Proliferation within Engineered Cartilaginous Tissue: the Role of Oxygen Tension. Biotechnol. Bioeng. 2005, 91 (5), 607-15.
98. Li. J., et al., Inhibition of reactive oxygen species by Lovas fa tin downregulates vascular endothelial growth factor expression and ameliorates blood-retinal barrier breakdown in db/db mice: role of NADPH oxidase 4. Diabetes, 2010. 59(6): p. 1528-38.
99. Li, J., et al., N-acetyl cysteine-loaded graphene oxide-collagen hybrid membrane for scarless wound healing. Theranostics, 2019. 9(20): p. 5839-5853.
100. Li, P., et al. Green and gentle synthesis of Cu20 nanoparticles using lignin as reducing and capping reagent with antibacterial properties. Journal of Experimental Nanoscience 11, 18-27 (2016).
101. Li, Y., et al., Antimicrobial Peptide-Inspired Design of Amino-Modified Lignin with Improved Antimicrobial Activities. Biomacromolecules, 2023. 24(11): p. 5381-5393.
102. Li, Z.; et al, Injectable, Highly Flexible, and Thermosensitive Hydrogels Capable of Delivering Superoxide Dismutase. Biomacromolecules 2009, JO (12), 3306-16.
103. Liu. R., et al., Lignin-Based Micro- and Nanomaterials and their Composites in Biomedical Applications, ChemSusChem, 2020. 13(17): p. 4266-4283.
104. Lou, H., et al. Effect of lignosulfonates on bacteria reproductions in recirculated cooling water. Chemistry and industry of Forest Products 24, 37-40 (2004).
105. Lv, X. et al. Structural and functional evaluation of oxygenating keratin/silk fibroin scaffold and initial assessment of their potential for urethral tissue engineering. Biomaterials 84, 99-110, doi:10.1016/j.biomaterials.2016.01.032 (2016).
106. Mabata, D.; et al, Lignin-graft-Polyoxazoline Conjugated Triazole a Novel Anti-Infective Ointment to Control Persistent Inflammation. Sci. Rep. 2017, 7, 46412.
107. Man Ha, C.; et al, Ectopic Defense Gene Expression is Associated with Growth Defects in Medicago truncatula Lignin Pathway Mutants. Plant Physiol. 2019, 181 (1), 63-84.
108. Manicourt, D. H., et al. Levels of circulating collagenase, stromelysin-1, and tissue inhibitor of matrix metalloproteinases 1 in patients with rheumatoid arthritis. Relationship to serum levels of antigenic keratan sulfate and systemic parameters of inflammation. Arthritis Rheum
38. 1031-1039, doi:10.1002/art.1780380803 (1995).
109. Martinez-Ferrer, M., et al., Dermal transforming growth factor-beta responsiveness mediates wound contraction and epithelial closure. Am J Pathol, 2010. 176(1): p. 98-107.
110. Mascharak, S.; et al, Preventing Engrailed-1 Activation in Fibroblasts Yields Wound Regeneration Without Scarring. Science 2021, 372 (6540).
111. Mazure, N. M., et al., Induction of vascular endothelial growth factor by hypoxia is modulated by a phosphatidylinositol 3-kinase/Akt signaling pathway in Ha-ras transiormed cells through a hypoxia inducible factor-I transcriptional element. Blood, 1997. 90(9): p. 3322-31.
112. Mehta, A. Y., et al., Allosterism-based simultaneous, dual anticoagulant and antiplatelet action: allosteric inhibitor targeting the glycoprotein Iba-binding and heparin-binding site of thrombin. Journal of Thrombosis and Haemostasis. 2016. 14(4): p. 828-838.
113. Moradipour, M. et al. Interaction of lignin dimers with model cell membranes: A quartz crystal microbalance and molecular dynamics simulation study. Biointerphases 16, 041003, doi:10.1116/6.0001029 (2021).
114. Moradipour, M. et al. Interaction of lignin-derived dimer and eugenol-functionalized silica nanoparticles with supported lipid bilayers. Colloids Surf B Biointerfaces 191, 111028, doi:10.1016/j.colsurfb.2020.111028 (2020).
115. Motzkau, M.; et al, Expression Of Matrix-Metalloproteases in The Fluid Of Chronic Diabetic Foot Wounds Treated With A Protease Absorbent Dressing. Exp. Clin. Endocrinol. Diabetes 2011, 119 (5), 286-290.
116. Nathan, D. M., et al., Intensive diabetes treatment and cardiovascular disease in patients with type I diabetes. N Engl J Med, 2005. 353(25): p. 2643-53.
117. New CDC report: More than JOO million Americans have diabetes or prediabetes, in Diabetes growth rate steady, adding to health care burden. 2017. Centers for Disease Control and Prevention.
118. Newland, B.; et al, Oxygen-Producing Gellan Gum Hydrogels for Dual Delivery of Either Oxygen or Peroxide with Doxorubicin. ACS Biomater. Sci. Eng. 2017, 3 (5), 787-792.
119. Newsholme, E. A.; et al. Regulation in Metabolism. 1973; Wiley Interscience; 349pp.
120. Nguyen, P. D., et al., Improved diabetic wound healing through topical silencing ofp53 is associated with augmented vasculogenic mediators. Wound Repair Regen, 2010. 18(6): p. 553-9.
121. Ninan, N., et al., Antibacterial and Anti-Inflammatory pH-Responsive Tannie Acid-Carboxylated Agarose Composite Hydrogels for Wound Healing. ACS Appl Mater Interfaces, 2016. 8(42): p. 28511-28521.
122. Nouvong, A., et al., Reactive oxygen species and bacterial biofilms in diabetic wound healing. Physiol Genomics, 20 16. 48(12): p. 889-896.
123. Novotortsev, V. M. et al. Examination of the disinfecting properties of calcium peroxide and its suitability for improving hydrodynamic characteristics of surface water bodies. Russian Journal of Applied Chemistry 85, 726-730, doi: 10.1134/S1070427212050072 (2012).
124. Nussbaum, S. R., et al., An Economic Evaluation of the Impact, Cost, and Medicare Policy Implications of Chronic Nonhealing Wounds. Value Health, 2018. 21(1): p. 27-32.
125. Olyaie, E.; et al. Development of a Cost-Effective Technique to Remove the Arsenic Contamination from Aqueous Solutions by Calcium Peroxide Nanoparticles. Sep. Purif. Technol. 2012, 95, 10-15.
126. Oveissi, F.; et al, Tough and Processable Hydrogels Based on Lignin and Hydrophilic Polyurethane. ACS Appl. Bio Mater. 2018, 1 (6), 2073-2081.
127. Padon, B. W. et al. Lignin Composites with Sustained Oxygenation and Reactive Oxygen Species-Scavenging Improve Neovascularization and Healing of Diabetic Wounds. bioRxiv, 2022.2006.2018.496670, doi: 10.1101/2022.06.18.496670 (2022).
128. Pan, X., et al., Organosolv ethanol lignin from hybrid poplar as a radical scavenger: Relationship between lignin structure, extraction conditions, and antioxidant activity. J. Agric. Food Chem., 2006. 54: p. 5806-5813.
129. Park, K. M., et al. The design of dextran-based hypoxia-inducible hydrogels via in situ oxygen-consuming reaction. Macromol Rapid Commun 35, 1968-1975, doi: 10.1002/marc.201400369 (2014).
130. Park, S.; et al, Hyperbaric Oxygen-Generating Hydrogels. Biomaterials 2018, 182, 234-244.
131. Qiu, M., et al., lignosulfonic Acid Exhibits Broadly Anti-HIV-/Activity—Potential as a Microbicide Candidate for the Prevention of HIV-I Sexual Transmission. PLOS ONE, 2012. 7(4):p. e35906.
132. Rao. C.; et al, Effects of Topical Oxygen Therapy on Ischemic Wound Healing. J Phys. Ther. Sci. 2016, 28 (1), 118-23.
133. Rastinfard, A., et al. Controlled chemical synthesis of CaO₂ particles coated with polyethylene glycol: characterization of crystallite size and oxygen release kinetics. RSC Advances 8, 91-101, doi: 10.1039/C7RA08758F (2018).
134. Ravishankar, K. et al. Biocompatible hydrogels of chitosan-alkali lignin for potential wound healing applications. Mater Sci Eng C Mater Biol Appl 102, 447-457, doi:10.1016/j.msec.2019.04.038 (2019).
135. Rendra, E.; et al, Reactive Oxygen Species (ROS) in Macrophage Activation and Function in Diabetes. Immunobiology 2019, 224 (2), 242-253.
136. Rossiter, H., et al., Loss of vascular endothelial growth factor α activity in murine epidermal keratinocytes delays wound healing and inhibits tumorfonnation. Cancer Res, 2004. 64(10): p. 3508-16.
137. Ryan, C. G., Quantitative Trace Element Imaging Using PIXE and the Nuclear Microprobe. Int. J. Imaging Sys !. Technol. 2000, 11 (4), 219-230.
138. Sacks, D., et al., Multisociety Consensus Quality Improvement Revised Consensus Statement for Endovascular Therapy of Acute Jschemic Stroke. Int J Stroke, 2018. 13(6): p. 612-632.
139. Samuel, E. L. G., et al., Hydrophilic carbon clusters as therapeutic, high-capacity antioxidants. Trends in biotechnology, 2014. 32(10): p. 501-505.
140. Sasso, F. C., et al., increased vascular endothelial growth factor expression but impaired vascular endothelial growth factor receptor signaling in the myocardium of type 2 diabetic patients with chronic coronary heart disease. J Am Coll Cardiol, 2005. 46(5): p. 827-34.
141. Schafer, M. et al, Oxidative stress in normal and impaired wound repair. Phannacol Res, 2008. 58(2): p. 165-71.
142. Sen, C. K. et al, Redox signals in wound healing. Biochim Biophys Acta, 2008. 1780(11): p. 1348-61.
143. Sharma, S. K. et al, In vitro antioxidant and free radical scavenging activity of Nardostachys jatamansi DC. J Acupunct Meridian Stud, 2012. 5(3): p. 112-8.
144. Sheikh, A. Y., et al., Effect of hyperoxia on vascular endothelial growth factor levels in a 145. Shen. S.; et al. Synthesis of CaO(2) Nanocrystals and Their Spherical Aggregates with Uniform Sizes for Use as a Biodegradable Bacteriostatic Agent. Small 2019, 15 (36), e l 902118.
146. Siwik, D. A., et al, Oxidative stress regulates collagen synthesis and matrix metalloproteinase activity in cardiac fibroblasts. Arn J Physiol Cell Physiol, 2001. 280(1): p. C53-60.
147. Son, D.; et al, Tailoring Physical Properties of Dual-Network Acrylamide Hydrogel Composites by Engineering Molecular Structures of the Cross-linked Network. ACS Omega 2022, 7 (34), 30028-30039.
148. Steiling, H.; et al, Different Types of ROS-scavenging Enzymes Are Expressed During Cutaneous Wound Repair. Exp. Cell Res. 1999, 247 (2): 484-94.
149. Suvamapathaki, S.; et al, Breathing Life into Engineered Tissues Using Oxygen-Releasing Biomaterials. NPG Asia Mater. 2019, 11 (1), 65.
150. Suvarnapathaki, S., et al. Engineering calcium peroxide based oxygen generating scaffolds for tissue survival. Biomater Sci 9, 2519-2532, doi: 10.1039/d0bm02048f (2021).
151. Suvarnapathaki, S.; et al, Oxygen-Generating Scalfolds for Cardiac Tissue Engineering Applications. ACS Biomater. Sci. Eng. 2022, 9 (1), 409-426.
152. Targosz-Korecka, M., et al., Stilfness memory of EA.hy926 endothelial cells in response to chronic hyperglycemia. Cardiovasc Diabetol, 2013, 12: p. 96.
153. Theocharidis, et al, A Strain-Programmed Patch for the Healing of Diabetic Wounds. Nat. Biomed. Eng. 2022, 6 (10), 111 8-1133.
154. Thi, P. L.; et al, Calcium Peroxide-Mediated In Situ Formation of Multifunctional Hydrogels with Enhanced Mesenchymal Stem Cell Behaviors and Antibacterial Properties. J Mater. Chem. B 2020, 8 (48), 11033-11043.
155. Thi, P. L.; et al, In Situ Forming and Reactive Oxygen Species-Scavenging Gelatin Hydrogels for Enhancing Wound Healing Efficacy. Acta Biomater. 2020, 103, 142-152.
156. Thiem, K., et al., Hyperglycemic Memory of Innate Immune Cells Promotes In Vitro Pro-inflammatory Responses of Human Monocytes and Murine Macrophages. J Immunol, 2021. 206(4): p. 807-813.
157. Todorciuc. T., et al. Adsorption of Cu (11) from aqueous solution on wheat straw lignin: Equilibrium and kinetic studies. Ce//Chem Technol 49, 439-447 (2015).
158. Tong, X. et al. Experimental and Molecular Dynamics Simulation Study of the Effects of Lignin Dimers on the Gel-to-Fluid Phase Transition in DPPC Bilayers. J Phys Chem B 123, 8247-8260, doi:10.1021/acs.jpcb.9b05525 (2019).
159. Tonino, P. et al. Microvessel Density Is Associated with VEGF and a-SMA Expression in Different Regions of Human Gastrointestinal Carcinomas. Cancers (Basel) 3, 3405-3418, doi: 10.3390/cancers3033405 (2011).
160. Tortora, M. et al. Ultrasound driven assembly of lignin into microcapsules for storage and delivery of hydrophobic molecules. Biomacromolecules 15, 1634-1643, doi:10.1021/bm500015j (2014).
161. Trengove, N.J., et al., Analysis of the acute and chronic wound environments: the role of proteases and their inhibitors. Wound Repair Regen, 1999. 7(6): p. 442-52.
162. Waite, A. J.; et al, Kinetics and Stoichiometry of Oxygen Release from Solid Peroxides. Environ. Eng. Sci. 1999, 16 (3), 187-199.
163. Waldron, A. L., et al., Oxidative stress-dependent MMP-13 activity underlies glucose neurotoxicity. J Diabetes Complications, 2018. 32(3): p. 249-257.
164. Wang, C.; et al, CaO(2)/Gelalin Oxygen Slow-Releasing Microspheres Facilitate Tissue Engineering Efficiency for the Osteonecrosis of Femoral Head by Enhancing the Angiogenesis and Survival of Grafted Bone Marrow Mesenchymal Stem Cells. Biomater. Sci. 2021, 9 (8), 3005-3018.
165. Wang. H.; et al Properties of Calcium Peroxide for Release of Hydrogen Peroxide and Oxygen: a Kinetics Study. Chem. Eng. J. 2016, 303, 450-457.
166. Wang. J., et al., Engineering PCL-Ilignin nanofibers as an antioxidant scaffold for the growth of neuron and Schwann cell. Colloids Surf B Biointerfaces, 2018. 169: p. 356-365.
167. Wang, R. M.; et al, Myocardial Matrix Hydrogel Acts as a Reactive Oxygen Species Scavenger and Supports a Proliferative Microenvironment for Cardiomyocytes. Acta Biomater. 2022, 152, 47-59.
168. Wilgus, T. A., Vascular Endothelial Growth Factor and Cutaneous Scarring. Adv. Wound Care 2019, 8 (12), 671-678.
169. Wu, H.; et al, Ceria Nanocrystals Decorated Mesoporous Silica Nanoparticle Based Ros-Scavenging Tissue Adhesive for Highly Efficient Regenerative Wound Healing. Biomaterials 2018, 151, 66-77.
170. Wysocki, J., et al., The influence of thymus extracts on the chemotaxis of polymorphonuclear neutrophils (PMNJ from patients with insulin-dependent diabetes mellitus (IDD). Thymus, 1992. 20(1): p. 63-7.
171. Xu. J.; et al, Lignin-Incorporated Nanogel Serving as an Antioxidant Biomaterial for Wound Healing. ACS Appl. Bio Mater. 2021, 4 (1): 3-13.
172. Xue, M.; et al, Extracellular Matrix Reorganization During Wound Healing and Its Impact on Abnormal Scarring. Adv. Wound Care 2015, 4 (3), 11 9-136.
173. Yang, W., et al., Polyvinyl alcohol chitosan hydrogels with enhanced antioxidant and antibacterial properties induced by lignin nanoparticles. Carbohydrate Polymers, 2018. 181: p. 275-284.
174. Yano, K., et al, Control of hair growth and follicle size by VEGFmediated angiogenesis. J Clin Invest, 2001. 107(4): p. 409-17.
175. Yekrang-Salakar, A; et al, Development of Rolled Scaffold for High-Density Adherent Cell Culture. Biomed. Microdevices 2019, 22 (1), 4.
176. Zeng. W.; et al, Antioxidant Treatment Enhances Human Mesenchymal Stem Cell Anti-Stress Ability and Therapeutic Efficacy in an Acute Liver Failure Model. Sci. Rep. 2015, 5, 11100.
177. Zhai, J. et al. in Advanced Materials Research. 648-653 (Trans Tech Publ).
178. Zhang, S.; et al, An Antioxidant Nanodrug Protects Against Hepatic Ischemia-Reperfusion Injury by Attenuating Oxidalive Stress and Inflammation. J. Mater. Chem. B 2022, JO (37), 7563-7569.
179. Zhang, Y., et al, Increased production of vascular endothelial growth factor in the lesions of atopic dermatitis. Arch Dermatol Res, 2006. 297(9): p. 425-9.
180. Zhang, Y., et al., Novel lignin-chilosan·PVA composite hydrogel for wound dressing. Mater Sci Eng C Mater Biol Appl, 2019. 104: p. 110002.
181. Zhao, H.; et al, Ros-Scavenging Hydrogel to Promote Healing of Bacteria Infected Diabetic Wounds. Biomaterials 2020, 258, 120286.
182. Zhuang, Z.; et al, Control of Matrix Stiffness Using Methacrylate-Gelatin Hydrogels for a Macrophage-Mediated Inflammatory Response. ACS Biomater. Sci. Eng. 2020, 6 (5), 3091-3102.
183. Zou, J. et al. Green and sustainable self-assembly nanocomposite from gentamicin sulfate/lignosulfonate with efficient antibacterial and wound-healing activity. ACS Sustainable Chemistry & Engineering 8, 4931-4940 (2020).

What is claimed is:

1. A composition comprising:
(a) one or more lignin derivatives;
(b) at least one inorganic peroxide; and
(c) a pharmaceutically-acceptable carrier.

2. The composition of claim 1, wherein the one or more lignin derivatives comprise thiolated lignosulfonate (TLS), sodium lignosulfonate (SLS), aminoalkyl TLS, or any combination thereof.

3. The composition of claim 2, wherein the TLS comprises from about 0.5 to about 6 mmol of thiol groups per gram of TLS.

4. The composition of claim 1, wherein the pharmaceutically-acceptable carrier comprises a hydrogel.

5. The composition of claim 1, wherein the pharmaceutically-acceptable carrier comprises methacrylated gelatin (GelMA).

6. The composition of claim 1, wherein the at least one inorganic peroxide comprises CaO₂, MgO₂, or any combination thereof.

7. The composition of claim 1, wherein the at least one inorganic peroxide is encapsulated in a core of a core-shell nanoparticle.

8. The composition of claim 7, wherein a shell of the core-shell nanoparticle comprises a functionalized lignin grafted to more polymers or copolymers.

9. The composition of claim 8, wherein the functionalized lignin comprises SLS.

10. The composition of claim 8, wherein the one or more polymers or copolymers comprises poly(lactic-co-glycolic) acid (PLGA), polycaprolactone (PCL), or any combination thereof.

11. The composition of claim 1, wherein the composition is substantially free of cytotoxic metal ions.

12. The composition of claim 1, wherein the composition is substantially free of unmodified gelatin.

13. A method for treating a wound in a subject, the method comprising administering the composition of claim 1 to the subject.

14. The method of claim 13, wherein the composition is administered to the subject topically, by injection, by aerosolization, using droplets, or any combination thereof.

15. The method of claim 13, wherein the subject is a mammal.

16. The method of claim 15, wherein the mammal is a human, cat, dog, rat, mouse, rabbit, hamster, guinea pig, or pig.

17. The method of claim 13, wherein the subject has diabetes, hypertension, obesity, a polymicrobial infection, systemic inflammation, or any combination thereof.

18. The method of claim 13, wherein performing the method enhances at least one wound healing response relative to an untreated wound.

19. The method of claim 18, wherein the at least one wound healing response comprises increased tissue granulation, increased neovascularization, reduced inflammatory response, reduced scarring, or any combination thereof.

20. The method of claim 13, wherein the at least one inorganic peroxide is encapsulated in a core of a core-shell nanoparticle; wherein a shell of the core-shell nanoparticle comprises a functionalized lignin grafted to more polymers or copolymers; wherein the one or more polymers or copolymers comprises poly(lactic-co-glycolic) acid (PLGA), polycaprolactone (PCL), or any combination thereof; and wherein the shell of the core-shell nanoparticle continues to scavenge reactive oxygen species (ROS) after depletion of the inorganic peroxide.
